SEQUENCE LISTING (25 sequences)

SEQ ID NO: 1 - DNA, 26 bp, synthetic construct
  source 1..26: mol_type = other DNA
ccaaaggctcttctcagagccaccca

SEQ ID NO: 2 - protein, 321 aa, synthetic construct
MPKKKRKVGGSLPSIEQMLAANPGKTPISLLQEYGTRIGKTPVYDLLKAEGQAHQPNFTFRVTVGDTSCTGQGPSKKAAKHKAAEVALKHLKGGSMLEPALEDSSSFSPLDSSLPEDIPVFTAAAAATPVPSVVLTRSPPMELQPPVSPQQSECNPVGALQELVVQKGWRLPEYTVTQESGPAHRKEFTMTCRVERFIEIGSGTSKKLAKRNAAAKMLLRVHTGGSGGSGGSGGSGGSGGSADFETDESVLMRRQKQINYGKNTIAYDRYIKEVPRHLRQPGIHPKTPNKFKKYSRRSWDQQIKLWKVALHFWDPKKKRKV

SEQ ID NO: 3 - protein, 321 aa, synthetic construct
MPKKKRKVADFETDESVLMRRQKQINYGKNTIAYDRYIKEVPRHLRQPGIHPKTPNKFKKYSRRSWDQQIKLWKVALHFWDGGSGGSGGSGGSGGSGGSGGSLPSIEQMLAANPGKTPISLLQEYGTRIGKTPVYDLLKAEGQAHQPNFTFRVTVGDTSCTGQGPSKKAAKHKAAEVALKHLKGGSMLEPALEDSSSFSPLDSSLPEDIPVFTAAAAATPVPSVVLTRSPPMELQPPVSPQQSECNPVGALQELVVQKGWRLPEYTVTQESGPAHRKEFTMTCRVERFIEIGSGTSKKLAKRNAAAKMLLRVHTPKKKRKV

SEQ ID NO: 4 - DNA, 10 bp, synthetic construct
  source 1..10: mol_type = other DNA
cgagctctct

SEQ ID NO: 5 - DNA, 10 bp, synthetic construct
  source 1..10: mol_type = other DNA
aacgagctct

SEQ ID NO: 6 - DNA, 10 bp, synthetic construct
  source 1..10: mol_type = other DNA
cgcaacgagc

SEQ ID NO: 7 - DNA, 10 bp, synthetic construct
  source 1..10: mol_type = other DNA
tatcgcaacg

SEQ ID NO: 8 - DNA, 10 bp, synthetic construct
  source 1..10: mol_type = other DNA
aataatatcg

SEQ ID NO: 9 - DNA, 10 bp, synthetic construct
  source 1..10: mol_type = other DNA
taagagagct

SEQ ID NO: 10 - DNA, 10 bp, synthetic construct
  source 1..10: mol_type = other DNA
aagagagctc

SEQ ID NO: 11 - DNA, 14 bp, synthetic construct
  source 1..14: mol_type = other DNA
agagagctcgttgc

SEQ ID NO: 12 - DNA, 10 bp, synthetic construct
  source 1..10: mol_type = other DNA
gagagctcgt

SEQ ID NO: 13 - DNA, 14 bp, synthetic construct
  source 1..14: mol_type = other DNA
agagctcgttgcga

SEQ ID NO: 14 - DNA, 10 bp, synthetic construct
  source 1..10: mol_type = other DNA
gagctcgttg

SEQ ID NO: 15 - RNA, 10 nt, synthetic construct
  source 1..10: mol_type = other RNA
cgagctctct

SEQ ID NO: 16 - RNA, 10 nt, synthetic construct
  source 1..10: mol_type = other RNA
aacgagctct

SEQ ID NO: 17 - RNA, 10 nt, synthetic construct
  source 1..10: mol_type = other RNA
cgcaacgagc

SEQ ID NO: 18 - RNA, 10 nt, synthetic construct
  source 1..10: mol_type = other RNA
tatcgcaacg

SEQ ID NO: 19 - RNA, 10 nt, synthetic construct
  source 1..10: mol_type = other RNA
aataatatcg

SEQ ID NO: 20 - RNA, 10 nt, synthetic construct
  source 1..10: mol_type = other RNA
taagagagct

SEQ ID NO: 21 - RNA, 10 nt, synthetic construct
  source 1..10: mol_type = other RNA
aagagagctc

SEQ ID NO: 22 - RNA, 14 nt, synthetic construct
  source 1..14: mol_type = other RNA
agagagctcgttgc

SEQ ID NO: 23 - RNA, 10 nt, synthetic construct
  source 1..10: mol_type = other RNA
gagagctcgt

SEQ ID NO: 24 - RNA, 14 nt, synthetic construct
  source 1..14: mol_type = other RNA
agagctcgttgcga

SEQ ID NO: 25 - RNA, 10 nt, synthetic construct
  source 1..10: mol_type = other RNA
gagctcgttg

(19) United States
(12) Patent Application Publication
(10) Pub. No.: US 2026/0028381 A1
(43) Pub. Date: Jan. 29, 2026

(54) SYSTEMS AND METHODS FOR PROMOTING TRANS-SPLICING

(71) Applicant: Tacit Therapeutics, Inc., South San Francisco, CA (US)

(72) Inventor: David Allen NELLES, South San Francisco, CA (US)

(21) Appl. No.: 18/994,856

(22) PCT Filed: May 19, 2023
(86) PCT No.: PCT/US2023/023007

§ 371 Date: Jan. 15, 2025

Related U.S. Application Data
(60) Provisional application No. 63/368,871, filed on Jul. 19, 2022.

(51) Int. Cl. C07K 14/47; C12N 15/113; C12N 15/86

(52) U.S. Cl. CPC C07K 14/4702; C12N 15/113; C12N 15/86; C07K 2319/85; C12N 2750/14143; C12N 2830/48

(57) ABSTRACT

Described herein are compositions systems and methods for promoting trans-splicing. In some examples, the system may comprise a nucleic acid molecule. The nucleic acid molecule may encode an exonic sequence. The system may further comprise a tethering fusion protein. The tethering fusion protein may promote an association of the exonic sequence and a target RNA or portion thereof.

### CROSS-REFERENCE

This application is a U.S. national state application under 35 U.S.C. § 371 of International Application No. PCT/US2023/023007, filed internationally on May 19, 2023 which claims the benefit of U.S. Provisional Application No. 63/368,871, filed Jul. 19, 2022, each of which is entirely incorporated herein by reference.

### STATEMENT AS TO FEDERALLY SPONSORED RESEARCH

This invention was made with government support under Contract 2112383 awarded by the National Science Foundation. The government has certain rights in the invention.

### REFERENCE TO AN ELECTRONIC SEQUENCE LISTING

The contents of the electronic sequence listing (309222000600SEQLIST.xml; Size: 23,430 bytes; and Date of Creation: Jan. 13, 2025) is herein incorporated by reference in its entirety.

### BACKGROUND

Effective treatment of human genetic disease may require efficient replacement of defective genetic sequences in human cells. Examples of human gene therapies include RNA trans-splicing.

### SUMMARY

Recognized herein is an industry-wide need for the creation of effective and efficient treatments that address the underlying cause of human genetic diseases.

An aspect of the present disclosure provides a system for trans-splicing, comprising: (a) a nucleic acid molecule encoding: (i) an exonic sequence; (ii) at least one intronic domain configured to promote insertion of said exonic sequence into a target RNA molecule; and (iii) one or more binding domains configured to interact with an RNA-binding protein, wherein said RNA-binding protein is encoded by one or more human-derived sequences; and (b) said RNA binding protein, which is configured to insert said exonic sequence into said target RNA molecule. In some embodiments, said intronic domain comprises said one or more binding domains. In some embodiments, the RNA binding protein is a tethering protein. In some embodiments, said tethering protein comprises: (a) an RNA-binding domain that binds to a specific sequence in said nucleic acid molecule; and (b) a non-specific double-stranded RNA binding domain that stabilizes the RNA-RNA hybridization between said nucleic acid molecule and said target RNA. In some embodiments, said non-specific double-stranded RNA binding domain comprises sequences isolated or derived from a gene selected from the group consisting of DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, said tethering protein further comprises a domain configured to associate with an enzyme configured to insert said exonic sequence into said target mRNA molecule. In some embodiments, said tethering protein further comprises an RNA-binding domain configured to bind a specific sequence in said nucleic acid molecule. In some embodiments, said tethering protein is isolated or derived from human protein sequences. In some embodiments, the system for trans-splicing further comprises an engineered small nuclear RNA derived or isolated from a U1 snRNA gene that promotes trans-splicing between a target RNA and said nucleic acid molecule. In some embodiments, said nucleic acid molecule comprises one or more binding sites for the RNA-binding domain. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter. In some embodiments, said system for trans-splicing lacks a CRISPR-associated protein. The present disclosure provides a vector comprising any one of the systems for trans-splicing as disclosed herein. In some embodiments, said vector is selected from the group consisting of: adeno-associated virus, retrovirus, lentivirus, adenovirus, nanoparticle, micelle, liposome, lipoplex, polymersome, polyplex, and dendrimer. The present disclosure provides a cell comprising any of the vectors as disclosed herein. The present disclosure provides a method for treating a disease comprising administering to a patient in need of a therapeutically effective amount of a treatment comprising any one of the systems for trans-splicing as disclosed herein. The present disclosure provides a method for correcting a genetic defect in a subject comprising administering to said subject any one of the systems for trans-splicing as disclosed herein.

Another aspect of the present disclosure provides a system for trans-splicing, comprising: (a) a nucleic acid molecule encoding: (i) an exonic sequence; and (ii) at least one intronic domain configured to promote insertion of said exonic sequence into a target RNA molecule; and (b) a protein configured to insert said exonic sequence into said target RNA molecule, wherein said system for trans-splicing lacks a CRISPR-associated protein. In some embodiments, said nucleic acid molecule comprises one or more binding sites for said protein. In some embodiments, said protein is a tethering protein. In some embodiments, said tethering protein is a fusion tethering protein. In some embodiments, said tethering protein comprises: (a) an RNA-binding domain that binds to a specific sequence in said nucleic acid molecule; and (b) a domain configured to associate with a transcriptional or spliceosomal protein. In some embodiments, said tethering protein further comprises a domain that binds non-specifically to double-stranded RNA that stabilizes the RNA-RNA hybridization between said nucleic acid molecule and said target RNA. In some embodiments, said tethering protein comprises: (a) an RNA-binding domain that binds to a specific sequence in said nucleic acid molecule; and (b) a non-specific double-stranded RNA binding domain that stabilizes the RNA-RNA hybridization between said nucleic acid molecule and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, said tethering protein is isolated or derived from human protein sequences. In some embodiments, the system for trans-splicing further comprises an engineered small nuclear RNA derived or isolated from a U1 snRNA gene that promotes trans-splicing between a target RNA and said nucleic acid molecule. In some embodiments, said nucleic acid molecule comprises one or more binding sites for the RNA-binding domain. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter. The present disclosure provides a vector comprising any one of the systems for trans-splicing as disclosed herein. In some embodiments, said vector is selected from the group consisting of: adeno-associated virus, retrovirus, lentivirus, adenovirus, nanoparticle, micelle, liposome, lipoplex, polymersome, polyplex, and dendrimer. The present disclosure provides a cell comprising any of the vectors as disclosed herein. The present disclosure provides a method for treating a disease comprising administering to a patient in need of a therapeutically effective amount of a treatment comprising any one of the systems for trans-splicing as disclosed herein. The present disclosure provides a method for correcting a genetic defect in a subject comprising administering to said subject any one of the systems for trans-splicing as disclosed herein.

Another aspect of the present disclosure provides a nucleic acid molecule encoding: (a) an exonic sequence; (b) at least one intronic domain configured to promote insertion of said exonic sequence into a target RNA molecule; and (c) one or more binding domains that interact with an RNA-binding protein, wherein said RNA-binding protein is encoded by one or more human-derived sequences, and wherein said RNA-binding protein is configured to interact with a transcriptional or spliceosomal enzyme coupled to said target RNA. In some embodiments, said system for trans-splicing lacks a CRISPR-associated protein. In some embodiments, said RNA-binding protein is a tethering protein. In some embodiments, said tethering protein is a tethering fusion protein. In some embodiments, said tethering protein further comprises an RNA-binding domain that binds to a specific sequence in said nucleic acid molecule. In some embodiments, said tethering protein comprises: (a) an RNA-binding domain that binds to a specific sequence in said nucleic acid molecule; and (b) a transport domain that associates with a transcriptional or spliceosomal enzyme coupled to said target RNA. In some embodiments, the transport domain comprises sequences isolated or derived from a gene involved in transcription, mediator complex, and/or the spliceosome. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, said tethering protein further comprises a domain that binds non-specifically to double-stranded RNA that stabilizes the RNA-RNA hybridization between said nucleic acid molecule and said target RNA. In some embodiments, said tethering protein further comprises an RNA-binding domain configured to bind a specific sequence in said nucleic acid molecule. In some embodiments, said tethering protein is isolated or derived from human protein sequences. In some embodiments, the system for trans-splicing further comprises an engineered small nuclear RNA derived or isolated from a U1 snRNA gene that promotes trans-splicing between a target RNA and said nucleic acid molecule. In some embodiments, said nucleic acid molecule comprises one or more binding sites for the RNA-binding domain. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter. Aspects of the present disclosure provide a vector comprising any one of the systems for trans-splicing as disclosed herein. In some embodiments, the vector is selected from the group consisting of: adeno-associated virus, retrovirus, lentivirus, adenovirus, nanoparticle, micelle, liposome, lipoplex, polymersome, polyplex, and dendrimer. Aspects of the present disclosure provide a cell comprising any one of the vectors disclosed herein. Aspects of the present disclosure provide a method for treating a disease comprising administering to a patient in need of a therapeutically effective amount of a treatment comprising any one of the systems for trans-splicing as disclosed herein. Aspects of the present disclosure provide a method for correcting a genetic defect in a subject comprising administering to said subject any one of the systems for trans-splicing as disclosed herein.

Another aspect of the present disclosure provides a system for trans-splicing, comprising: (a) a nucleic acid molecule encoding: (i) an exonic sequence; and (ii) at least one intronic domain configured to promote insertion of said exonic sequence into a target RNA molecule; and (b) an RNA-binding protein configured to interact with a transcriptional or spliceosomal enzyme coupled to said target RNA, wherein said system for trans-splicing lacks a CRISPR-associated protein. In some embodiments, said nucleic acid molecule further comprises one or more binding sites configured to bind said RNA-binding protein. In some embodiments, said RNA-binding protein is a tethering protein. In some embodiments, said tethering protein is a tethering fusion protein. In some embodiments, said tethering protein further comprises an RNA-binding domain that binds to a specific sequence in said nucleic acid molecule. In some embodiments, said tethering protein comprises: (a) an RNA-binding domain that binds to a specific sequence in said nucleic acid molecule; and (b) a transport domain that associates with a transcriptional or spliceosomal enzyme coupled to said target RNA. In some embodiments, said transport domain comprises sequences isolated or derived from a gene involved in transcription, the mediator complex, and/or the spliceosome. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, said tethering protein further comprises a domain that binds non-specifically to double-stranded RNA that stabilizes the RNA-RNA hybridization between said nucleic acid molecule and said target RNA. In some embodiments, said domain that binds non-specifically to double-stranded RNA comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said tethering protein is isolated or derived from human protein sequences. In some embodiments, the system for trans-splicing further comprises an engineered small nuclear RNA derived or isolated from a U1 snRNA gene that promotes trans-splicing between a target RNA and said nucleic acid molecule. In some embodiments, said nucleic acid molecule comprises one or more binding sites for the RNA-binding domain. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter. Aspects of the present disclosure provide a vector comprising any one of the systems for trans-splicing as disclosed herein. In some embodiments, said vector is selected from the group consisting of: adeno-associated virus, retrovirus, lentivirus, adenovirus, nanoparticle, micelle, liposome, lipoplex, polymersome, polyplex, and dendrimer. The present disclosure provides a cell comprising any one of the vectors as disclosed herein. The present disclosure provides a method for treating a disease comprising administering to a patient in need of a therapeutically effective amount of a treatment comprising any one of the systems for trans-splicing as disclosed herein. The present disclosure provides a method for correcting a genetic defect in a subject comprising administering to said subject any one of the systems for trans-splicing as disclosed herein.

Another aspect of the present disclosure provides a system for trans-splicing, comprising: (a) a nucleic acid molecule encoding: (i) an exonic sequence; (ii) at least one intronic domain configured to promote insertion of said exonic sequence into a target RNA molecule; and (iii) one or more binding domains that interact with an RNA-binding protein, wherein said RNA-binding protein is encoded by one or more human-derived sequences; and (b) a tethering protein that promotes the association of said exonic sequence and said target RNA molecule, and wherein said tethering protein is configured to bind to said one or more binding domains. In some embodiments, said tethering protein is a fusion protein. In some embodiments, said tethering protein comprises: (a) an RNA-binding domain that binds to a specific sequence in said nucleic acid molecule; and (b) a domain configured to associate with a transcriptional or spliceosomal protein. In some embodiments, said tethering protein comprises: (a) an RNA-binding domain that binds to a specific sequence in said nucleic acid molecule; and (b) a non-specific double-stranded RNA binding domain that stabilizes the RNA-RNA hybridization between said nucleic acid molecule and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, the tethering protein further comprises a domain that binds non-specifically to double-stranded RNA that stabilizes the RNA-RNA hybridization between said nucleic acid molecule and said target RNA. In some embodiments, said domain that binds non-specifically to double-stranded RNA comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said tethering protein further comprises an RNA-binding domain configured to bind a specific sequence in said nucleic acid molecule. In some embodiments, said tethering protein comprises: (a) an RNA-binding domain configured to bind a specific sequence in said nucleic acid molecule; and (b) a domain configured to associate with a transcriptional or spliceosomal complex. In some embodiments, the system for trans-splicing further comprises an engineered small nuclear RNA derived or isolated from a U1 snRNA gene that promotes trans-splicing between a target RNA and said nucleic acid molecule. In some embodiments, said nucleic acid molecule comprises one or more binding sites for the RNA-binding domain. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter. The present disclosure provides a vector comprising any one of the systems for trans-splicing as disclosed herein. In some embodiments, said vector is selected from the group consisting of: adeno-associated virus, retrovirus, lentivirus, adenovirus, nanoparticle, micelle, liposome, lipoplex, polymersome, polyplex, and dendrimer. The present disclosure provides a cell comprising any of the vectors as disclosed herein. The present disclosure provides a method for treating a disease comprising administering to a patient in need of a therapeutically effective amount of a treatment comprising any one of the systems for trans-splicing as disclosed herein. The present disclosure provides a method for correcting a genetic defect in a subject comprising administering to said subject any one of the systems for trans-splicing as disclosed herein.

Another aspect of the present disclosure provides a system for trans-splicing, comprising: (a) a nucleic acid molecule encoding: (i) an exonic sequence; and (ii) at least one intronic domain configured to promote insertion of said exonic sequence into a target RNA molecule; and (b) a tethering protein that promotes the association of said exonic sequence and said target RNA molecule, wherein said system for trans-splicing lacks a CRISPR-associated enzyme. In some embodiments, the tethering protein is configured to bind to one or more binding sites in said nucleic acid molecule. In some embodiments, said tethering protein is a fusion protein. In some embodiments, said tethering protein is an RNA-binding fusion protein. In some embodiments, said tethering protein comprises: (a) an RNA-binding domain that binds to a specific sequence in said nucleic acid molecule; and (b) a domain configured to associate with a transcriptional or spliceosomal protein. In some embodiments, said tethering protein comprises: (a) an RNA-binding domain that binds to a specific sequence in said nucleic acid molecule; and (b) a non-specific double-stranded RNA binding domain that stabilizes the RNA-RNA hybridization between said nucleic acid molecule and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, the tethering protein further comprises a domain that binds non-specifically to double-stranded RNA that stabilizes the RNA-RNA hybridization between said nucleic acid molecule and said target RNA. In some embodiments, said domain that binds non-specifically to double-stranded RNA comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said tethering protein further comprises an RNA-binding domain configured to bind a specific sequence in said nucleic acid molecule. In some embodiments, said tethering protein comprises: (a) an RNA-binding domain configured to bind a specific sequence in said nucleic acid molecule; and (b) a domain configured to associate with a transcriptional or spliceosomal complex. In some embodiments, said tethering protein is isolated or derived from human protein sequences. In some embodiments, the system for trans-splicing further comprises an engineered small nuclear RNA derived or isolated from a U1 snRNA gene that promotes trans-splicing between a target RNA and said nucleic acid molecule. In some embodiments, said nucleic acid molecule comprises one or more binding sites for the RNA-binding domain. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter. The present disclosure provides a vector comprising any one of the systems for trans-splicing as disclosed herein. In some embodiments, said vector is selected from the group consisting of: adeno-associated virus, retrovirus, lentivirus, adenovirus, nanoparticle, micelle, liposome, lipoplex, polymersome, polyplex, and dendrimer. The present disclosure provides a cell comprising any of the vectors as disclosed herein. The present disclosure provides a method for treating a disease comprising administering to a patient in need of a therapeutically effective amount of a treatment comprising any one of the systems for trans-splicing as disclosed herein. The present disclosure provides a method for correcting a genetic defect in a subject comprising administering to said subject any one of the systems for trans-splicing as disclosed herein.

Another aspect of the present disclosure provides a method of associating an exonic sequence with a target RNA, the method comprising: (a) providing a nucleic acid encoding: (i) an exonic sequence; (ii) at least one intronic domain configured to promote insertion of said exonic sequence into a target RNA molecule; and (iii) one or more binding domains that interact with a tethering protein; and (b) binding a tethering protein to said one or more binding domains and to said target RNA molecule to associate said exonic sequence with said target RNA molecule. In some embodiments, said tethering protein is a fusion protein. In some embodiments, said method is performed in the absence of a CRISPR-associated enzyme. In some embodiments, said tethering protein comprises: (a) an RNA-binding domain that binds to a specific sequence in said nucleic acid molecule; and (b) a domain configured to associate with a transcriptional or spliceosomal protein. In some embodiments, said tethering protein comprises: (a) an RNA-binding domain that binds to a specific sequence in said nucleic acid molecule; and (b) a non-specific double-stranded RNA binding domain that stabilizes the RNA-RNA hybridization between said nucleic acid molecule and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, the method further comprises providing a enzyme configured to insert said exonic sequence into said target RNA molecule. In some embodiments, said tethering protein further comprises a domain configured to associate with said enzyme configured to insert said exonic sequence into said target RNA molecule. In some embodiments, said tethering protein further comprises an RNA-binding domain configured to bind a specific sequence in said nucleic acid molecule. In some embodiments, said tethering protein comprises: (a) an RNA-binding domain configured to bind a specific sequence in said nucleic acid molecule; and (b) a domain configured to associate with a transcriptional or spliceosomal complex. In some embodiments, said tethering protein is isolated or derived from human protein sequences. In some embodiments, the method further comprises providing an engineered small nuclear RNA derived or isolated from a U1 snRNA gene that promotes trans-splicing between a target RNA and said nucleic acid molecule. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter.

Another aspect of the present disclosure provides a method of associating an exonic sequence with a target RNA, the method comprising: (a) providing a nucleic acid molecule encoding: (i) a exonic sequence; and (ii) at least one intronic domain configured to promote insertion of said exonic sequence into a target RNA molecule; and (b) binding a tethering protein to said target RNA molecule and said exonic sequence to associate said exonic sequence with said target RNA molecule, wherein said trans-splicing molecule does not associate with a CRISPR enzyme. In some embodiments, said tethering protein is a fusion tethering protein. In some embodiments, said tethering protein comprises: (a) an RNA-binding domain that binds to a specific sequence in said nucleic acid molecule; and (b) a domain configured to associate with a transcriptional or spliceosomal protein. In some embodiments, said tethering protein comprises: (a) an RNA-binding domain that binds to a specific sequence in said nucleic acid molecule; and (b) a non-specific double-stranded RNA binding domain that stabilizes the RNA-RNA hybridization between said nucleic acid molecule and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, the method further comprises providing a enzyme configured to insert said exonic sequence into said target RNA molecule. In some embodiments, said tethering protein further comprises a domain configured to associate with said enzyme configured to insert said exonic sequence into said target RNA molecule. In some embodiments, said tethering protein comprises: (a) an RNA-binding domain configured to bind a specific sequence in said nucleic acid molecule; and (b) a domain configured to associate with a transcriptional or spliceosomal complex. In some embodiments, said tethering protein is isolated or derived from human protein sequences. In some embodiments, the method further comprises providing an engineered small nuclear RNA derived or isolated from a U1 snRNA gene that promotes trans-splicing between a target RNA and said nucleic acid molecule. In some embodiments, said nucleic acid molecule comprises one or more binding sites for the RNA-binding domain. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter.

Another aspect of the present disclosure provides a method for trans-splicing, comprising: (a) providing a nucleic acid molecule encoding: (i) a exonic sequence; (ii) at least one intronic domain configured to promote insertion of said exonic sequence into a target RNA molecule; and (iii) one or more binding domains that interact with an RNA-binding protein, wherein said one or more binding domains are encoded by one or more human-derived sequences; and using an enzyme to insert said exonic sequence into a target RNA molecule. In some embodiments, said RNA-binding protein is a tethering protein. In some embodiments, said tethering protein is a fusion tethering protein. In some embodiments, said tethering protein further comprises a domain configured to associate with said enzyme configured to insert said exonic sequence into said target RNA molecule. In some embodiments, said tethering protein comprises: (a) an RNA-binding domain that binds to a specific sequence in said nucleic acid molecule; and (b) a domain configured to associate with a transcriptional or spliceosomal protein. In some embodiments, said tethering protein comprises: (a) an RNA-binding domain that binds to a specific sequence in said nucleic acid molecule; and (b) a non-specific double-stranded RNA binding domain that stabilizes the RNA-RNA hybridization between said nucleic acid molecule and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, the method further comprises an engineered small nuclear RNA derived or isolated from a U1 snRNA gene that promotes trans-splicing between a target RNA and said nucleic acid molecule. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter.

Another aspect of the present disclosure provides a method for trans-splicing, comprising: (a) providing a nucleic acid molecule encoding: (i) a exonic sequence; and (ii) at least one intronic domain configured to promote insertion of said exonic sequence into a target RNA molecule; and (b) using an enzyme to insert said exonic sequence into a target RNA molecule, wherein said nucleic acid molecule does not associate with a CRISPR enzyme. In some embodiments, the method further comprises a tethering protein. In some embodiments, said tethering protein comprises: (a) an RNA-binding domain that binds to a specific sequence in said nucleic acid molecule; and (b) a domain configured to associate with a transcriptional or spliceosomal protein. In some embodiments, said tethering protein comprises: (a) an RNA-binding domain that binds to a specific sequence in said nucleic acid molecule; and (b) a non-specific double-stranded RNA binding domain that stabilizes the RNA-RNA hybridization between said nucleic acid molecule and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, said tethering protein further comprises a domain configured to associate with said enzyme configured to insert said exonic sequence into said target RNA molecule. In some embodiments, said tethering protein further comprises an RNA-binding domain configured to bind a specific sequence in said nucleic acid molecule. In some embodiments, said tethering protein is isolated or derived from human protein sequences. In some embodiments, the method further comprises an engineered small nuclear RNA derived or isolated from a U1 snRNA gene that promotes trans-splicing between a target RNA and said nucleic acid molecule. In some embodiments, said nucleic acid molecule comprises one or more binding sites for the RNA-binding domain. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter.

Another aspect of the present disclosure provides a method for trans-splicing, comprising: (a) providing a nucleic acid molecule encoding: (i) a exonic sequence; and (ii) at least one intronic domain comprising a site that interacts with an RNA-binding protein, wherein said RNA-binding protein comprises a domain configured to interact with a transcriptional or spliceosomal enzyme coupled to said target RNA, and wherein said RNA-binding protein is derived from one or more human-derived sequences; and (b) interacting said RNA-binding protein with said transcriptional or spliceosomal enzyme. In some embodiments, said RNA-binding protein is a tethering protein. In some embodiments, said tethering protein comprises: (a) an RNA-binding domain that binds to a specific sequence in said nucleic acid molecule; and (b) a non-specific double-stranded RNA binding domain that stabilizes the RNA-RNA hybridization between said nucleic acid molecule and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, said tethering protein further comprises a domain configured to associate with said enzyme configured to insert said exonic sequence into said target RNA molecule. In some embodiments, said tethering protein further comprises an RNA-binding domain configured to bind a specific sequence in said nucleic acid molecule. In some embodiments, said tethering protein is isolated or derived from human protein sequences. In some embodiments, the method further comprises an engineered small nuclear RNA derived or isolated from a U1 snRNA gene that promotes trans-splicing between a target RNA and said nucleic acid molecule. In some embodiments, said nucleic acid molecule comprises one or more binding sites for the RNA-binding domain. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter. In some embodiments, said nucleic acid molecule does not interact with a CRISPR enzyme.

Another aspect of the present disclosure provides a method for trans-splicing, comprising: (a) providing a nucleic acid molecule encoding: (i) a exonic sequence; and (ii) at least one intronic domain comprising a site that interacts with an RNA-binding protein, wherein said RNA-binding protein comprises a domain configured to interact with a transcriptional or spliceosomal enzyme coupled to said target RNA; and (b) interacting said RNA-binding protein with said transcriptional or spliceosomal enzyme, wherein said system for trans-splicing lacks a CRISPR-associated protein. In some embodiments, the method further comprises providing a tethering protein. In some embodiments, said tethering protein comprises: (a) an RNA-binding domain that binds to a specific sequence in said nucleic acid molecule; and (b) a non-specific double-stranded RNA binding domain that stabilizes the RNA-RNA hybridization between said nucleic acid molecule and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule comprises sequences isolated or derived from a PUF or Pumby protein. In some embodiments, said RNA-binding domain that binds to a specific sequence encoded by the nucleic acid molecule comprises sequences isolated or derived from the gene SLBP. In some embodiments, said tethering protein further comprises a domain configured to associate with said enzyme configured to insert said exonic sequence into said target RNA molecule. In some embodiments, said tethering protein further comprises an RNA-binding domain configured to bind a specific sequence in said nucleic acid molecule. In some embodiments, said tethering protein is isolated or derived from human protein sequences. In some embodiments, the method further comprises an engineered small nuclear RNA derived or isolated from a U1 snRNA gene that promotes trans-splicing between a target RNA and said nucleic acid molecule. In some embodiments, said nucleic acid molecule comprises one or more binding sites for the RNA-binding domain. In some embodiments, said nucleic acid molecule further comprises a sequence that promotes accumulation of the nucleic acid molecule in the cellular nucleus. In some embodiments, said sequence that promotes accumulation of the nucleic acid molecule in the cellular nucleus is derived or isolated from a long noncoding RNA. In some embodiments, said nucleic acid molecule further comprises a 3′ untranslated region that increases the stability of the exonic sequence. In some embodiments, said nucleic acid molecule further comprises a 5′ untranslated region that increases the stability of the exonic sequence. In some embodiments, said nucleic acid molecule further comprises a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further comprises a heterologous promoter.

Another aspect of the present disclosure provides a system for trans-splicing comprising a nucleic acid encoding an exonic sequence and a tethering fusion protein, wherein the tethering fusion protein promotes the association of the exonic sequence and a target RNA. In some embodiments, the tethering fusion protein comprises: (a) an RNA-binding domain that binds to a specific sequence encoded by the nucleic acid molecule; and (b) a non-specific double-stranded RNA binding domain that stabilizes the RNA-RNA hybridization among the nucleic acid molecule and target RNA. In some embodiments, the non-specific double-stranded RNA binding domain comprises sequences isolated or derived from a gene selected from the group consisting of DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, the RNA-binding domain that binds to a specific sequence encoded by the nucleic acid molecule comprises sequences isolated or derived from a PUF or Pumby protein. In some embodiments, the RNA-binding domain that binds to a specific sequence encoded by the nucleic acid molecule comprises sequences isolated or derived from the gene SLBP. In some embodiments, the tethering fusion protein comprises: (a) an RNA-binding domain that binds a specific sequence encoded by the nucleic acid molecule; and (b) a domain that associates with the spliceosome. In some embodiments, the tethering fusion protein comprises: (a) an RNA-binding domain that binds a specific sequence encoded by the nucleic acid molecule; and (b) a domain that associates with a transcriptional or spliceosomal complex. In some embodiments, the tethering fusion protein is isolated or derived from human protein sequences. In some embodiments, the system for trans-splicing further comprises an engineered small nuclear RNA derived or isolated from the U1 snRNA gene that promotes trans-splicing among the target RNA and nucleic acid molecule. In some embodiments, the nucleic acid molecule comprises one or more binding sites for the RNA-binding domain. In some embodiments, the nucleic acid molecule further comprises a sequence promotes accumulation of the nucleic acid molecule in the cellular nucleus. In some embodiments, the sequence that promotes accumulation of the nucleic acid molecule in the cellular nucleus is derived or isolated from a long noncoding RNA. In some embodiments, the nucleic acid molecule further comprises a 3′ untranslated region that increases the stability of the exonic sequence. In some embodiments, the nucleic acid molecule further comprises a 5′ untranslated region that increases the stability of the exonic sequence. In some embodiments, the nucleic acid molecule further comprises a gene expression-enhancing element. In some embodiments, the gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, the nucleic acid molecule comprises RNA, DNA, a DNA/RNA hybrid, a nucleic acid analog, a chemically-modified nucleic acid, or a chimera composed of two or more nucleic acids or nucleic acid analogs. In some embodiments, the nucleic acid molecule further comprises a heterologous promoter. The present disclosure provides a vector comprising any one of the systems for trans-splicing as disclosed herein. In some embodiments, said vector is selected from the group consisting of: adeno-associated virus, retrovirus, lentivirus, adenovirus, nanoparticle, micelle, liposome, lipoplex, polymersome, polyplex, and dendrimer. The present disclosure provides a cell comprising any of the vectors as disclosed herein. The present disclosure provides a method for treating a disease comprising administering to a patient in need of a therapeutically effective amount of a treatment comprising any one of the systems for trans-splicing as disclosed herein. The present disclosure provides a method for correcting a genetic defect in a subject comprising administering to said subject any one of the systems for trans-splicing as disclosed herein.

Another aspect of the present disclosure provides a method of targeting an exonic sequence to a target RNA, the method comprising: (a) providing a nucleic acid encoding said exonic sequence; (b) providing said target RNA; and (c) using a tethering fusion protein to associate said exonic sequence and said target RNA. In some embodiments, the tethering fusion protein comprises: (a) an RNA-binding domain that binds to a specific sequence encoded by the nucleic acid molecule; and (b) a non-specific double-stranded RNA binding domain that stabilizes the RNA-RNA hybridization among the nucleic acid molecule and target RNA. In some embodiments, the non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, the RNA-binding domain that binds to a specific sequence encoded by the nucleic acid molecule comprises sequences isolated or derived from a PUF or Pumby protein. In some embodiments, the RNA-binding domain that binds to a specific sequence encoded by the nucleic acid molecule comprises sequences isolated or derived from the gene SLBP. In some embodiments, the tethering fusion protein comprises: (a) an RNA-binding domain that binds a specific sequence encoded by the nucleic acid molecule; and (b) a domain that associates with the spliceosome. In some embodiments, the tethering fusion protein comprises: (a) an RNA-binding domain that binds a specific sequence encoded by the nucleic acid molecule; and (b) a domain that associates with a transcriptional or spliceosomal complex. In some embodiments, the tethering fusion protein is isolated or derived from human protein sequences. In some embodiments, the method further comprises an engineered small nuclear RNA derived or isolated from the U1 snRNA gene that promotes trans-splicing among the target RNA and nucleic acid molecule. In some embodiments, the nucleic acid molecule comprises one or more binding sites for the RNA-binding domain. In some embodiments, the nucleic acid molecule further comprises a sequence promotes accumulation of the nucleic acid molecule in the cellular nucleus. In some embodiments, the sequence that promotes accumulation of the nucleic acid molecule in the cellular nucleus is derived or isolated from a long noncoding RNA. In some embodiments, the nucleic acid molecule further comprises a 3′ untranslated region that increases the stability of the exonic sequence. In some embodiments, the nucleic acid molecule further comprises a 5′ untranslated region that increases the stability of the exonic sequence. In some embodiments, the nucleic acid molecule further comprises a gene expression-enhancing element. In some embodiments, the gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, the nucleic acid molecule comprises RNA, DNA, a DNA/RNA hybrid, a nucleic acid analog, a chemically-modified nucleic acid, or a chimera composed of two or more nucleic acids or nucleic acid analogs. In some embodiments, the nucleic acid molecule comprises DNA. In some embodiments, the DNA is transcribed into an RNA molecule, wherein the RNA molecule is a trans-splicing RNA molecule. In some embodiments, the nucleic acid molecule further comprises a heterologous promoter. The present disclosure provides a vector comprising any one of the systems for trans-splicing as disclosed herein. In some embodiments, said vector is selected from the group consisting of: adeno-associated virus, retrovirus, lentivirus, adenovirus, nanoparticle, micelle, liposome, lipoplex, polymersome, polyplex, and dendrimer. The present disclosure provides a cell comprising any of the vectors as disclosed herein. The present disclosure provides a method for treating a disease comprising administering to a patient in need of a therapeutically effective amount of a treatment comprising any one of the systems for trans-splicing as disclosed herein. The present disclosure provides a method for correcting a genetic defect in a subject comprising administering to said subject any one of the systems for trans-splicing as disclosed herein.

Another aspect of the present disclosure provides a system for trans-splicing, comprising: a trans-splicing ribonucleic acid (RNA) molecule comprising: a replacement domain; and at least one intronic domain configured to promote insertion of said replacement domain into a target RNA molecule; and one or more binding domains configured to interact with an RNA-binding protein, wherein said RNA-binding protein is encoded by one or more human-derived sequences; and said RNA binding protein, which is configured to insert said replacement domain into said target RNA molecule. In some embodiments, said intronic domain comprises said one or more binding domains. In some embodiments, the RNA binding protein is a tethering protein. In some embodiments, said tethering protein comprises: (a) an RNA-binding domain that binds to a specific sequence encoded by the nucleic acid molecule; and (b) a non-specific double-stranded RNA binding domain that stabilizes the RNA-RNA hybridization between said specific sequence and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, said tethering protein further comprises a domain configured to associate with an enzyme configured to insert said replacement domain into said target mRNA molecule. In some embodiments, said tethering protein further comprises an RNA-binding domain configured to bind a specific sequence encoded by the nucleic acid molecule. In some embodiments, said tethering protein is isolated or derived from human protein sequences. In some embodiments, the system for trans-splicing further comprises an engineered small nuclear RNA derived or isolated from a U1 snRNA gene that promotes trans-splicing between a target RNA and said exonic sequence. In some embodiments, said nucleic acid molecule encodes one or more binding sites for the RNA-binding domain. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter. In some embodiments, said system for trans-splicing lacks a CRISPR-associated protein.

Another aspect of the disclosure provides a vector comprising the system for trans-splicing as disclosed herein. In some embodiments, said vector is selected from the group consisting of adeno-associated virus, retrovirus, lentivirus, adenovirus, nanoparticle, micelle, liposome, lipoplex, polymersome, polyplex, and dendrimer.

Another aspect of the disclosure provides a cell comprising the vector as disclosed herein.

Another aspect of the disclosure provides a method for treating a disease comprising administering to a patient in need of a therapeutically effective amount of a treatment comprising a trans-splicing nucleic acid molecule as disclosed herein.

Another aspect of the present disclosure provides a system for trans-splicing, comprising: a trans-splicing ribonucleic acid (RNA) molecule comprising: a replacement domain; and at least one intronic domain configured to promote insertion of said replacement domain into a target RNA molecule; and a protein configured to insert said replacement domain into said target RNA molecule, wherein said system for trans-splicing lacks a CRISPR-associated protein. In some embodiments, said nucleic acid molecule encodes one or more binding sites for said protein. In some embodiments, said protein is a tethering protein. In some embodiments, said tethering protein is a fusion tethering protein. In some embodiments, said tethering protein comprises: (a) an RNA-binding domain that binds to a specific sequence encoded by the nucleic acid molecule; and (b) a domain configured to associate with a transcriptional or spliceosomal protein. In some embodiments, said tethering protein further comprises a domain that binds non-specifically to double-stranded RNA that stabilizes the RNA-RNA hybridization between said specific sequence and said target RNA. In some embodiments, said tethering protein comprises: (a) an RNA-binding domain that binds to a specific sequence encoded by the nucleic acid molecule; and (b) a non-specific double-stranded RNA binding domain that stabilizes the RNA-RNA hybridization between said specific sequence and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, said tethering protein is isolated or derived from human protein sequences. In some embodiments, the system for trans-splicing further comprises an engineered small nuclear RNA derived or isolated from a U1 snRNA gene that promotes trans-splicing between a target RNA and said exonic sequence. In some embodiments, said nucleic acid molecule encodes one or more binding sites for the RNA-binding domain. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter. A vector comprising the system for trans-splicing of claim 25. In some embodiments, said vector is selected from the group consisting of adeno-associated virus, retrovirus, lentivirus, adenovirus, nanoparticle, micelle, liposome, lipoplex, polymersome, polyplex, and dendrimer.

Another aspect of the present disclosure provides a cell comprising the vector as disclosed herein.

Another aspect of the present disclosure provides a method for treating a disease comprising administering to a patient in need of a therapeutically effective amount of a treatment comprising a trans-splicing nucleic acid molecule as disclosed herein.

Another aspect of the present disclosure provides a method for correcting a genetic defect in a subject comprising administering to said subject a trans-splicing nucleic acid molecule as disclosed herein.

Another aspect of the present disclosure provides a trans-splicing ribonucleic acid (RNA) molecule comprising: a replacement domain; at least one intronic domain configured to promote insertion of said replacement domain into a target RNA molecule; and one or more binding domains that interact with an RNA-binding protein, wherein said RNA-binding protein is encoded by one or more human-derived sequences, and wherein said RNA-binding protein is configured to interact with a transcriptional or spliceosomal enzyme coupled to said target RNA. In some embodiments, said system for trans-splicing lacks a CRISPR-associated protein. In some embodiments, said RNA-binding protein is a tethering protein. In some embodiments, said tethering protein is a tethering fusion protein. In some embodiments, said tethering protein further comprises an RNA-binding domain that binds to a specific sequence encoded by the nucleic acid molecule. In some embodiments, said tethering protein comprises: (a) an RNA-binding domain that binds to a specific sequence encoded by the nucleic acid molecule; and (b) a transport domain that associates with a transcriptional or spliceosomal enzyme coupled to said target RNA. In some embodiments, the transport domain comprises sequences isolated or derived from a gene involved in transcription, mediator complex, and/or the spliceosome. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, said tethering protein further comprises a domain that binds non-specifically to double-stranded RNA that stabilizes the RNA-RNA hybridization between said specific sequence and said target RNA. In some embodiments, said tethering protein further comprises an RNA-binding domain configured to bind a specific sequence encoded by the nucleic acid molecule. In some embodiments, said tethering protein is isolated or derived from human protein sequences. In some embodiments, the system for trans-splicing further comprises an engineered small nuclear RNA derived or isolated from a U1 snRNA gene that promotes trans-splicing between a target RNA and said exonic sequence. In some embodiments, said nucleic acid molecule encodes one or more binding sites for the RNA-binding domain. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter.

Another aspect of the present disclosure provides a vector comprising the system for trans-splicing as disclosed herein. In some embodiments, said vector is selected from the group consisting of: adeno-associated virus, retrovirus, lentivirus, adenovirus, nanoparticle, micelle, liposome, lipoplex, polymersome, polyplex, and dendrimer.

Another aspect of the present disclosure provides a cell comprising the vector as disclosed herein.

Another aspect of the present disclosure provides a method for treating a disease comprising administering to a patient in need of a therapeutically effective amount of a treatment comprising a trans-splicing nucleic acid molecule as disclosed herein.

Another aspect of the present disclosure provides a method for correcting a genetic defect in a subject comprising administering to said subject a trans-splicing nucleic acid molecule as disclosed herein.

Another aspect of the present disclosure provides a system for trans-splicing, comprising: a trans-splicing ribonucleic acid (RNA) molecule comprising: a replacement domain; and at least one intronic domain configured to promote insertion of said replacement domain into a target RNA molecule; and an RNA-binding protein configured to interact with a transcriptional or spliceosomal enzyme coupled to said target RNA, wherein said system for trans-splicing lacks a CRISPR-associated protein. In some embodiments, the nucleic acid molecule further encodes one or more binding sites configured to bind said RNA-binding protein. In some embodiments, said RNA-binding protein is a tethering protein. In some embodiments, said tethering protein is a tethering fusion protein. In some embodiments, said tethering protein further comprises an RNA-binding domain that binds to a specific sequence encoded by the nucleic acid molecule. In some embodiments, said tethering protein comprises: (a) an RNA-binding domain that binds to a specific sequence encoded by the nucleic acid molecule; and (b) a transport domain that associates with a transcriptional or spliceosomal enzyme coupled to said target RNA. In some embodiments, said transport domain comprises sequences isolated or derived from a gene involved in transcription, the mediator complex, and/or the spliceosome. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, said tethering protein further comprises a domain that binds non-specifically to double-stranded RNA that stabilizes the RNA-RNA hybridization between said specific sequence and said target RNA. In some embodiments, said domain that binds non-specifically to double-stranded RNA comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said tethering protein is isolated or derived from human protein sequences. In some embodiments, the system for trans-splicing further comprises an engineered small nuclear RNA derived or isolated from a U1 snRNA gene that promotes trans-splicing between a target RNA and said exonic sequence. In some embodiments, said nucleic acid molecule encodes one or more binding sites for the RNA-binding domain. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter.

Another aspect of the present disclosure provides a vector comprising the system for trans-splicing as disclosed herein. In some embodiments, said vector is selected from the group consisting of adeno-associated virus, retrovirus, lentivirus, adenovirus, nanoparticle, micelle, liposome, lipoplex, polymersome, polyplex, and dendrimer.

Another aspect of the present disclosure provides a cell comprising the vector as disclosed herein.

Another aspect of the present disclosure provides a method for treating a disease comprising administering to a patient in need of a therapeutically effective amount of a treatment comprising a trans-splicing nucleic acid molecule as disclosed herein.

Another aspect of the present disclosure provides a method for correcting a genetic defect in a subject comprising administering to said subject a trans-splicing nucleic acid molecule as disclosed herein.

Another aspect of the present disclosure provides a system for trans-splicing, comprising: a trans-splicing ribonucleic acid (RNA) molecule comprising: a replacement domain; at least one intronic domain configured to promote insertion of said replacement domain into a target RNA molecule; and one or more binding domains that interact with an RNA-binding protein, wherein said RNA-binding protein is encoded by one or more human-derived sequences; and a tethering protein that promotes the association of said trans-splicing RNA molecule and said target RNA molecule, and wherein said tethering protein is configured to bind to said one or more binding domains. In some embodiments, said tethering protein is a fusion protein. In some embodiments, said tethering protein comprises: (a) an RNA-binding domain that binds to a specific sequence encoded by the nucleic acid molecule; and (b) a domain configured to associate with a transcriptional or spliceosomal protein. In some embodiments, said tethering protein comprises: (a) an RNA-binding domain that binds to a specific sequence encoded by the nucleic acid molecule; and (b) a non-specific double-stranded RNA binding domain that stabilizes the RNA-RNA hybridization between said specific sequence and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, the tethering protein further comprises a domain that binds non-specifically to double-stranded RNA that stabilizes the RNA-RNA hybridization between said specific sequence and said target RNA. In some embodiments, said domain that binds non-specifically to double-stranded RNA comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said tethering protein further comprises an RNA-binding domain configured to bind a specific sequence encoded by the nucleic acid molecule. In some embodiments, said tethering protein comprises: (a) an RNA-binding domain configured to bind a specific sequence encoded by the nucleic acid molecule; and (b) a domain configured to associate with a transcriptional or spliceosomal complex. In some embodiments, the system for trans-splicing further comprises an engineered small nuclear RNA derived or isolated from a U1 snRNA gene that promotes trans-splicing between a target RNA and said exonic sequence. In some embodiments, said nucleic acid molecule encodes one or more binding sites for the RNA-binding domain. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter.

Another aspect of the present disclosure provides a vector comprising the system for trans-splicing as disclosed herein. In some embodiments, said vector is selected from the group consisting of adeno-associated virus, retrovirus, lentivirus, adenovirus, nanoparticle, micelle, liposome, lipoplex, polymersome, polyplex, and dendrimer.

Another aspect of the present disclosure provides a cell comprising the vector as disclosed herein.

Another aspect of the present disclosure provides a method for treating a disease comprising administering to a patient in need of a therapeutically effective amount of a treatment comprising a trans-splicing nucleic acid molecule as disclosed herein.

Another aspect of the present disclosure provides a method for correcting a genetic defect in a subject comprising administering to said subject a trans-splicing nucleic acid molecule as disclosed herein.

Another aspect of the present disclosure provides a system for trans-splicing, comprising: a trans-splicing ribonucleic acid (RNA) molecule comprising: a replacement domain; and at least one intronic domain configured to promote insertion of said replacement domain into a target RNA molecule; and a tethering protein that promotes the association of said trans-splicing RNA molecule and said target RNA molecule, wherein said system for trans-splicing lacks a CRISPR-associated enzyme. In some embodiments, the tethering protein is configured to bind to one or more binding sites in said trans-splicing RNA. In some embodiments, said tethering protein is a fusion protein. In some embodiments, said tethering protein is an RNA-binding fusion protein. In some embodiments, said tethering protein comprises: (a) an RNA-binding domain that binds to a specific sequence encoded by the nucleic acid molecule; and (b) a domain configured to associate with a transcriptional or spliceosomal protein. In some embodiments, said tethering protein comprises: (a) an RNA-binding domain that binds to a specific sequence encoded by the nucleic acid molecule; and (b) a non-specific double-stranded RNA binding domain that stabilizes the RNA-RNA hybridization between said specific sequence and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, the tethering protein further comprises a domain that binds non-specifically to double-stranded RNA that stabilizes the RNA-RNA hybridization between said specific sequence and said target RNA. In some embodiments, said domain that binds non-specifically to double-stranded RNA comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said tethering protein further comprises an RNA-binding domain configured to bind a specific sequence encoded by the nucleic acid molecule. In some embodiments, said tethering protein comprises: (a) an RNA-binding domain configured to bind a specific sequence encoded by the nucleic acid molecule; and (b) a domain configured to associate with a transcriptional or spliceosomal complex. In some embodiments, said tethering protein is isolated or derived from human protein sequences. In some embodiments, the system for trans-splicing further comprises an engineered small nuclear RNA derived or isolated from a U1 snRNA gene that promotes trans-splicing between a target RNA and said exonic sequence. In some embodiments, said nucleic acid molecule encodes one or more binding sites for the RNA-binding domain. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter.

Another aspect of the present disclosure provides a vector comprising the system for trans-splicing as disclosed herein. In some embodiments, said vector is selected from the group consisting of adeno-associated virus, retrovirus, lentivirus, adenovirus, nanoparticle, micelle, liposome, lipoplex, polymersome, polyplex, and dendrimer.

Another aspect of the present disclosure provides a cell comprising the vector as disclosed herein.

Another aspect of the present disclosure provides a method for treating a disease comprising administering to a patient in need of a therapeutically effective amount of a treatment comprising a trans-splicing nucleic acid molecule as disclosed herein.

Another aspect of the present disclosure provides a method for correcting a genetic defect in a subject comprising administering to said subject a trans-splicing nucleic acid molecule as disclosed herein.

Another aspect of the present disclosure provides a method of associating a trans-splicing ribonucleic acid (RNA) molecule with a target RNA, the method comprising: providing said trans-splicing RNA molecule comprising: a replacement domain; and at least one intronic domain configured to promote insertion of said replacement domain into a target RNA molecule; and one or more binding domains that interact with a tethering protein; and binding a tethering protein to said one or more binding domains and to said target RNA molecule to associate said trans-splicing RNA molecule with said target RNA molecule. In some embodiments, said tethering protein is a fusion protein. In some embodiments, said method is performed in the absence of a CRISPR-associated enzyme. In some embodiments, said tethering protein comprises: (a) an RNA-binding domain that binds to a specific sequence encoded by the nucleic acid molecule; and (b) a domain configured to associate with a transcriptional or spliceosomal protein. In some embodiments, said tethering protein comprises: (a) an RNA-binding domain that binds to a specific sequence encoded by the nucleic acid molecule; and (b) a non-specific double-stranded RNA binding domain that stabilizes the RNA-RNA hybridization between said specific sequence and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, the method further comprises providing a enzyme configured to insert said replacement domain into said target RNA molecule. In some embodiments, said tethering protein further comprises a domain configured to associate with said enzyme configured to insert said replacement domain into said target RNA molecule. In some embodiments, said tethering protein further comprises an RNA-binding domain configured to bind a specific sequence encoded by the nucleic acid molecule. In some embodiments, said tethering protein comprises: (a) an RNA-binding domain configured to bind a specific sequence encoded by the nucleic acid molecule; and (b) a domain configured to associate with a transcriptional or spliceosomal complex. In some embodiments, said tethering protein is isolated or derived from human protein sequences. In some embodiments, the method further comprises providing an engineered small nuclear RNA derived or isolated from a U1 snRNA gene that promotes trans-splicing between a target RNA and said trans-splicing RNA. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter.

Another aspect of the present disclosure provides a method of associating a trans-splicing ribonucleic acid (RNA) molecule with a target RNA, the method comprising: providing said trans-splicing RNA molecule, wherein said nucleic acid molecule encodes: a replacement domain; and at least one intronic domain configured to promote insertion of said replacement domain into a target RNA molecule; and binding a tethering protein to said target RNA molecule and said trans-splicing RNA molecule to associate said trans-splicing RNA molecule with said target RNA molecule, wherein said trans-splicing molecule does not associate with a CRISPR enzyme. In some embodiments, said tethering protein is a fusion tethering protein. In some embodiments, said tethering protein comprises: (a) an RNA-binding domain that binds to a specific sequence encoded by the nucleic acid molecule; and (b) a domain configured to associate with a transcriptional or spliceosomal protein. In some embodiments, said tethering protein comprises: (a) an RNA-binding domain that binds to a specific sequence encoded by the nucleic acid molecule; and (b) a non-specific double-stranded RNA binding domain that stabilizes the RNA-RNA hybridization between said specific sequence and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, the method further comprises providing a enzyme configured to insert said replacement domain into said target RNA molecule. In some embodiments, said tethering protein further comprises a domain configured to associate with said enzyme configured to insert said replacement domain into said target RNA molecule. In some embodiments, said tethering protein comprises: (a) an RNA-binding domain configured to bind a specific sequence encoded by the nucleic acid molecule; and (b) a domain configured to associate with a transcriptional or spliceosomal complex. In some embodiments, said tethering protein is isolated or derived from human protein sequences. In some embodiments, the method further comprises providing an engineered small nuclear RNA derived or isolated from a U1 snRNA gene that promotes trans-splicing between a target RNA and said trans-splicing RNA. In some embodiments, said nucleic acid molecule encodes one or more binding sites for the RNA-binding domain. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter.

Another aspect of the present disclosure provides a method for trans-splicing, comprising: providing a trans-splicing ribonucleic acid (RNA) molecule comprising: a replacement domain; at least one intronic domain configured to promote insertion of said replacement domain into a target RNA molecule; and one or more binding domains that interact with an RNA-binding protein, wherein said one or more binding domains are encoded by one or more human-derived sequences; and using an enzyme to insert said replacement domain into a target RNA molecule. In some embodiments, said RNA-binding protein is a tethering protein. In some embodiments, said tethering protein is a fusion tethering protein. In some embodiments, said tethering protein further comprises a domain configured to associate with said enzyme configured to insert said replacement domain into said target RNA molecule. In some embodiments, said tethering protein comprises: (a) an RNA-binding domain that binds to a specific sequence encoded by the nucleic acid molecule; and (b) a domain configured to associate with a transcriptional or spliceosomal protein. In some embodiments, said tethering protein comprises: (a) an RNA-binding domain that binds to a specific sequence encoded by the nucleic acid molecule; and (b) a non-specific double-stranded RNA binding domain that stabilizes the RNA-RNA hybridization between said specific sequence and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, the method further comprises an engineered small nuclear RNA derived or isolated from a U1 snRNA gene that promotes trans-splicing between a target RNA and said trans-splicing RNA. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter.

Another aspect of the present disclosure provides a method for trans-splicing, comprising: providing a trans-splicing ribonucleic acid (RNA) molecule comprising: a replacement domain; and at least one intronic domain configured to promote insertion of said replacement domain into a target RNA molecule; and using an enzyme to insert said replacement domain into a target RNA molecule, wherein said trans-splicing RNA molecule does not associate with a CRISPR enzyme. In some embodiments, the method further comprises a tethering protein. In some embodiments, said tethering protein comprises: (a) an RNA-binding domain that binds to a specific sequence encoded by the nucleic acid molecule; and (b) a domain configured to associate with a transcriptional or spliceosomal protein. In some embodiments, said tethering protein comprises: (a) an RNA-binding domain that binds to a specific sequence encoded by the nucleic acid molecule; and (b) a non-specific double-stranded RNA binding domain that stabilizes the RNA-RNA hybridization between said specific sequence and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, said tethering protein further comprises a domain configured to associate with said enzyme configured to insert said replacement domain into said target RNA molecule. In some embodiments, said tethering protein further comprises an RNA-binding domain configured to bind a specific sequence encoded by the nucleic acid molecule. In some embodiments, said tethering protein is isolated or derived from human protein sequences. In some embodiments, the method further comprises an engineered small nuclear RNA derived or isolated from a U1 snRNA gene that promotes trans-splicing between a target RNA and said trans-splicing RNA. In some embodiments, said nucleic acid molecule encodes one or more binding sites for the RNA-binding domain. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter.

Another aspect of the present disclosure provides a method for trans-splicing, comprising: providing a trans-splicing ribonucleic acid (RNA) molecule comprising: a replacement domain; and at least one intronic domain comprising a site that interacts with an RNA-binding protein, wherein said RNA-binding protein comprises a domain configured to interact with a transcriptional or spliceosomal enzyme coupled to said target RNA, and wherein said RNA-binding protein is derived from one or more human-derived sequences; and interacting said RNA-binding protein with said transcriptional or spliceosomal enzyme. In some embodiments, said RNA-binding protein is a tethering protein. In some embodiments, said tethering protein comprises: (a) an RNA-binding domain that binds to a specific sequence encoded by the nucleic acid molecule; and (b) a non-specific double-stranded RNA binding domain that stabilizes the RNA-RNA hybridization between said specific sequence and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, said tethering protein further comprises a domain configured to associate with said enzyme configured to insert said replacement domain into said target RNA molecule. In some embodiments, said tethering protein further comprises an RNA-binding domain configured to bind a specific sequence encoded by the nucleic acid molecule. In some embodiments, said tethering protein is isolated or derived from human protein sequences. In some embodiments, the method further comprises an engineered small nuclear RNA derived or isolated from a U1 snRNA gene that promotes trans-splicing between a target RNA and said trans-splicing RNA. In some embodiments, said nucleic acid molecule encodes one or more binding sites for the RNA-binding domain. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter. In some embodiments, said trans-splicing RNA molecule does not interact with a CRISPR enzyme.

Another aspect of the present disclosure provides a method for trans-splicing, comprising: providing a trans-splicing ribonucleic acid (RNA) molecule comprising: a replacement domain; and at least one intronic domain comprising a site that interacts with an RNA-binding protein, wherein said RNA-binding protein comprises a domain configured to interact with a transcriptional or spliceosomal enzyme coupled to said target RNA; and interacting said RNA-binding protein with said transcriptional or spliceosomal enzyme, wherein said system for trans-splicing lacks a CRISPR-associated protein. In some embodiments, the method further comprises providing a tethering protein. In some embodiments, said tethering protein comprises: (a) an RNA-binding domain that binds to a specific sequence encoded by the nucleic acid molecule; and (b) a non-specific double-stranded RNA binding domain that stabilizes the RNA-RNA hybridization between said specific sequence and said target RNA. In some embodiments, said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, said RNA-binding domain that binds to a specific sequence encoded by the nucleic acid molecule encodes sequences isolated or derived from a PUF or Pumby protein. In some embodiments, said RNA-binding domain that binds to a specific sequence encoded by the nucleic acid molecule encodes sequences isolated or derived from the gene SLBP. In some embodiments, said tethering protein further comprises a domain configured to associate with said enzyme configured to insert said replacement domain into said target RNA molecule. In some embodiments, said tethering protein further comprises an RNA-binding domain configured to bind a specific sequence encoded by the nucleic acid molecule. In some embodiments, said tethering protein is isolated or derived from human protein sequences. In some embodiments, the method further comprises an engineered small nuclear RNA derived or isolated from a U1 snRNA gene that promotes trans-splicing between a target RNA and said trans-splicing RNA. In some embodiments, said nucleic acid molecule encodes one or more binding sites for the RNA-binding domain. In some embodiments, said nucleic acid molecule further encodes a sequence that promotes accumulation of the exonic sequence in the cellular nucleus. In some embodiments, said sequence that promotes accumulation of the exonic sequence in the cellular nucleus is derived or isolated from a long noncoding RNA. In some embodiments, said nucleic acid molecule further encodes a 3′ untranslated region that increases the stability of the exonic sequence. In some embodiments, said nucleic acid molecule further encodes a 5′ untranslated region that increases the stability of the exonic sequence. In some embodiments, said nucleic acid molecule further encodes a gene expression-enhancing element. In some embodiments, said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, said nucleic acid molecule further encodes a heterologous promoter.

Another aspect of the present disclosure provides a system for trans-splicing comprising a trans-splicing RNA and a tethering fusion protein wherein the tethering fusion protein promotes the association of the trans-splicing RNA and a target RNA. In some embodiments, the tethering fusion protein comprises: (a) an RNA-binding domain that binds to a specific sequence in the trans-splicing RNA; and (b) a non-specific double-stranded RNA binding domain that stabilizes the RNA-RNA hybridization among the trans-splicing RNA and target RNA. In some embodiments, the non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, the RNA-binding domain that binds to a specific sequence in the trans-splicing RNA comprises sequences isolated or derived from a PUF or Pumby protein. In some embodiments, the RNA-binding domain that binds to a specific sequence in the trans-splicing RNA comprises sequences isolated or derived from the gene SLBP. In some embodiments, the tethering fusion protein comprises: (a) an RNA-binding domain that binds a specific sequence in the trans-splicing RNA; and (b) a domain that associates with the spliceosome. In some embodiments, the tethering fusion protein comprises: (a) an RNA-binding domain that binds a specific sequence in the trans-splicing RNA; and (b) a domain that associates with a transcriptional or spliceosomal complex. In some embodiments, the tethering fusion protein is isolated or derived from human protein sequences. In some embodiments, the system for trans-splicing further comprises an engineered small nuclear RNA derived or isolated from the U1 snRNA gene that promotes trans-splicing among the target RNA and trans-splicing RNA. In some embodiments, the trans-splicing RNA comprises one or more binding sites for the RNA-binding domain. In some embodiments, the trans-splicing RNA further encodes a sequence promotes accumulation of the trans-splicing RNA in the cellular nucleus. In some embodiments, the sequence that promotes accumulation of the trans-splicing RNA in the cellular nucleus is derived or isolated from a long noncoding RNA. In some embodiments, the trans-splicing RNA further encodes a 3′ untranslated region that increases the stability of the exonic sequence. In some embodiments, the trans-splicing RNA further encodes a 5′ untranslated region that increases the stability of the exonic sequence. In some embodiments, the trans-splicing RNA further encodes a gene expression-enhancing element. In some embodiments, the gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, the trans-splicing nucleic acid is RNA, DNA, a DNA/RNA hybrid, a nucleic acid analog, a chemically-modified nucleic acid, or a chimera composed of two or more nucleic acids or nucleic acid analogs. In some embodiments, the nucleic acid molecule further comprises a heterologous promoter.

Another aspect of the present disclosure provides a vector comprising the system for trans-splicing as disclosed herein. In some embodiments, the vector is selected from the group consisting of: adeno-associated virus, retrovirus, lentivirus, adenovirus, nanoparticle, micelle, liposome, lipoplex, polymersome, polyplex, and dendrimer.

Another aspect of the present disclosure provides a cell comprising the vector as disclosed herein.

Another aspect of the present disclosure provides a method for treating a disease comprising administering to a patient in need of a therapeutically effective amount of a treatment comprising a trans-splicing nucleic acid molecule as disclosed herein.

Another aspect of the present disclosure provides a method for correcting a genetic defect in a subject comprising administering to said subject a trans-splicing nucleic acid molecule as disclosed herein.

Another aspect of the present disclosure provides a method of targeting a trans-splicing ribonucleic acid (RNA) to a target RNA, the method comprising: providing said trans-splicing RNA; providing said target RNA; and using a tethering fusion protein to associate said trans-splicing RNA and said target RNA. In some embodiments, the tethering fusion protein comprises: (a) an RNA-binding domain that binds to a specific sequence in the trans-splicing RNA; and (b) a non-specific double-stranded RNA binding domain that stabilizes the RNA-RNA hybridization among the trans-splicing RNA and target RNA. In some embodiments, the non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1. In some embodiments, the RNA-binding domain that binds to a specific sequence in the trans-splicing RNA comprises sequences isolated or derived from a PUF or Pumby protein. In some embodiments, the RNA-binding domain that binds to a specific sequence in the trans-splicing RNA comprises sequences isolated or derived from the gene SLBP. In some embodiments, the tethering fusion protein comprises: (a) an RNA-binding domain that binds a specific sequence in the trans-splicing RNA; and (b) a domain that associates with the spliceosome. In some embodiments, the tethering fusion protein comprises: (a) an RNA-binding domain that binds a specific sequence in the trans-splicing RNA; and (b) a domain that associates with a transcriptional or spliceosomal complex. In some embodiments, the tethering fusion protein is isolated or derived from human protein sequences. In some embodiments, the method further comprises an engineered small nuclear RNA derived or isolated from the U1 snRNA gene that promotes trans-splicing among the target RNA and trans-splicing RNA. In some embodiments, the trans-splicing RNA comprises one or more binding sites for the RNA-binding domain. In some embodiments, the trans-splicing RNA further encodes a sequence promotes accumulation of the trans-splicing RNA in the cellular nucleus. In some embodiments, the sequence that promotes accumulation of the trans-splicing RNA in the cellular nucleus is derived or isolated from a long noncoding RNA. In some embodiments, the trans-splicing RNA further encodes a 3′ untranslated region that increases the stability of the exonic sequence. In some embodiments, the trans-splicing RNA further encodes a 5′ untranslated region that increases the stability of the exonic sequence. In some embodiments, the trans-splicing RNA further encodes a gene expression-enhancing element. In some embodiments, the gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, the trans-splicing nucleic acid is RNA, DNA, a DNA/RNA hybrid, a nucleic acid analog, a chemically-modified nucleic acid, or a chimera composed of two or more nucleic acids or nucleic acid analogs. In some embodiments, the nucleic acid molecule further comprises a heterologous promoter.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1E illustrates an exemplary mechanism by which tethered trans-splicing can treat a genetic disease. FIG. 1A illustrates the concept of human genetic disease where mutated (“defective”) DNA sequences are transcribed into RNA which directly contribute to disease (“RNA pathogenicity”) or are translated into disease-causing protein (“translation of pathogenic protein”). FIG. 1B illustrates an exemplary tethered trans-splicing as described herein. In this example, a mutation-carrying RNA molecule is targeted by a trans-splicing RNA that corrects the mutation with high efficiency. The tethering fusion protein may promote efficient editing by promoting association of the trans-splicing RNA and the target RNA. FIG. 1C further illustrates an exemplary mechanism wherein the tethering mechanism promotes RNA editing with high efficiency. In this example, the tethering fusion promotes the association of the trans-splicing RNA and the target RNA by simultaneously associating with both RNA molecules. One domain of the tethering fusion protein (the “specific RNA binding domain”) associates with a specific sequence within the trans-splicing RNA. A second domain (the “non-specific RNA binding domain”) associates with the RNA-RNA duplex among the target RNA and the trans-splicing RNA. FIG. 1D illustrates another exemplary method by which the tethering fusion protein promotes association of the target RNA and trans-splicing RNA. In this example, one domain of the tethering fusion protein (the “spliceosome-component binding protein”) associates with a spliceosome enzyme that is located in close proximity to the target RNA. FIG. 1E illustrates another exemplary method by which the tethering fusion protein promotes association of the trans-splicing RNA and target RNA. In this example, the tethering fusion protein includes a domain that binds to a transcriptional enzyme (the “transcriptional component binding protein”).

FIGS. 2A-2C illustrate the results of an exemplary tethered trans-splicing system on the composition of a target RNA. FIG. 2A describes an exemplary double trans-splicing RNA which carries two antisense domains, one replacement domain, two intronic domains, and at least one tethering fusion protein that associates with the 5′ and 3′ ends of the trans-splicing RNA. This design may promote replacement of an internal sequence within the target RNA while maintaining the adjacent 5′ and 3′ sequences around the replaced sequence. FIGS. 2B-2C describe exemplary terminal trans-splicing RNAs that both comprise one antisense domain, one replacement domain, one intronic domain, and at least one tethering fusion protein. FIG. 2B illustrates the exemplary design of a 3′ terminal trans-splicing RNA that replaces the 3′ terminal end of a target RNA while maintaining the 5′ end. FIG. 2C illustrates an exemplary design of a 5′ terminal trans-splicing molecule that replaces the 5′ terminal end of a target RNA while maintaining the 3′ end.

FIGS. 3A-3D illustrate the design of an experiment testing the role of the tethering fusion protein in the context of internal trans-splicing via production of GFP protein. FIG. 3A illustrates an exemplary design of a split GFP reporter that carries N- and C-terminal portions of GFP (“N-GFP” and “C-GFP”) but lacks an internal GFP sequence required for fluorescence. In the reporter, this internal sequence is replaced by a short exon with a stop codon that is flanked by introns. The internal sequence (“int-GFP”) is the replacement sequence within an RNA trans-splicing molecule that is flanked by two intronic sequences and two antisense sequences. The absence of the RNA trans-splicing molecule or tethering fusion protein (FIGS. 3B-3C) results in cis-splicing primarily. Addition of the tethering fusion protein results in increased trans-splicing and therefore increased GFP signal (FIG. 3D).

FIGS. 4A-4D illustrate the design of an experiment testing the role of the tethering fusion protein in the context of 5′ terminal trans-splicing. FIG. 4A illustrates an exemplary design of a split GFP reporter that carries a C-terminal portion of GFP (“C-GFP”) but lacks an N-terminal GFP sequence required for fluorescence. In the reporter, this N-terminal GFP sequence is replaced by a short exon with a stop codon that is flanked by introns. The N-terminal sequence (“N-GFP”) is the replacement sequence within an RNA trans-splicing molecule that is flanked by one intronic sequence and one antisense sequence. The absence of the RNA trans-splicing molecule or tethering fusion protein (FIGS. 4B-4C) results in cis-splicing primarily. Specifically, in FIG. 4C, the lack of a tethering protein results in cis-splicing primarily and, as a result, low GFP signal, because the tethering protein may promote association of the trans-splicing molecule to the target RNA sequence. Thus, lack of the tethering protein may result in reduced association of the trans-splicing molecule to the target RNA sequence, thereby resulting in primarily cis-splicing. Addition of the tethering fusion protein results in increased trans-splicing and therefore increased GFP signal (FIG. 4D). The tethering protein may promote association of the trans-splicing molecule to the target RNA sequence. Thus, inclusion of the tethering protein may result in increased association of the trans-splicing molecule to the target RNA sequence, thereby resulting in primarily trans-splicing.

FIGS. 5A-5D illustrate the design of an experiment testing the role of the tethering fusion protein in the context of 3′ terminal trans-splicing. FIG. 5A illustrates the design of a split GFP reporter that carries a N-terminal portion of GFP (“N-GFP”) but lacks an C-terminal GFP sequence required for fluorescence. In the reporter, this C-terminal GFP sequence is replaced by a short exon with a stop codon that is flanked by introns. The C-terminal sequence (“C-GFP”) is the replacement sequence within an RNA trans-splicing molecule that is flanked by one intronic sequence and one antisense sequence. The absence of the RNA trans-splicing molecule or tethering fusion protein (FIGS. 5B-5C) results in cis-splicing primarily. Specifically, in FIG. 5C, the lack of a tethering protein results in cis-splicing primarily and, as a result, low GFP signal, because the tethering protein may promote association of the trans-splicing molecule to the target RNA sequence. Thus, lack of the tethering protein may result in reduced association of the trans-splicing molecule to the target RNA sequence, thereby resulting in primarily cis-splicing. Addition of the tethering fusion protein results in increased trans-splicing and therefore increased GFP signal (FIG. 5D). The tethering protein may promote association of the trans-splicing molecule to the target RNA sequence. Thus, inclusion of the tethering protein may result in increased association of the trans-splicing molecule to the target RNA sequence, thereby resulting in primarily trans-splicing.

FIGS. 6A-6B illustrate the potential for single or multiple tethering fusion protein binding sites. By including one or more specific RNA binding domains in the trans-splicing RNA, the RNA-RNA duplex among the trans-splicing RNA and target RNA can be stabilized further.

FIGS. 7A-F illustrate empirical data collected that describe the ability of tethering fusion proteins to enhance trans-splicing activity. FIGS. 7A, C, D are schematics of a trans-splicing molecule that comprise varied numbers binding sites for Stem-Loop Binding Protein (SLBP). FIGS. 7B, D, F illustrate data collected using a reporter assay similar to that which is described in FIG. 4 involving various tethering fusion proteins comprising SLBP. In summary, FIG. 7 illustrates that SLBP fused to the double-stranded RNA binding domains of transactivation response element RNA-binding protein (TRBP) increases trans-splicing activity the most compared to other tethering fusion proteins and to a control trans-splicing RNA that lacks SLBP binding sites. The figure legend describes whether a trans-splicing RNA is present (“+” indicates that a trans-splicing molecule with SLBP sites is present, “+*” indicates a trans-splicing molecule lacking SLBP sites is present, and “−” indicates that no trans-splicing molecule is present). The figure legend also describes the identities of the N- and C-terminal portions of the tethering fusion protein where parenthetical amino acid numbering indicates that a specific portion of the referenced protein is present.

FIGS. 8A-F illustrate empirical data collected that describe the ability of tethering fusion proteins to enhance trans-splicing activity. FIGS. 8A, C, D are schematics of a trans-splicing molecule that comprise varied numbers binding sites for mPum1. FIGS. 8B, D, F illustrate data collected using a reporter assay similar to that which is described in FIG. 4 involving various tethering fusion proteins comprising mPum1. In summary, FIG. 8 illustrates that mPum1 fused to the double-stranded RNA binding domains of TRBP increases trans-splicing activity the most compared to other tethering fusion proteins and to a control trans-splicing RNA that lacks mPum1 binding sites. The figure legend describes whether a trans-splicing RNA is present (“+” indicates that a trans-splicing molecule with mPum1 sites is present, “+*” indicates a trans-splicing molecule lacking mPum1 sites is present, and “−” indicates that no trans-splicing molecule is present). The figure legend also describes the identities of the N- and C-terminal portions of the tethering fusion protein where parenthetical amino acid numbering indicates that a specific portion of the referenced protein is present.

FIGS. 9A-F illustrate empirical data collected that describe the ability of tethering fusion proteins to enhance trans-splicing activity. FIGS. 9A, C, and D are schematics of a trans-splicing molecule that comprise varied numbers binding sites for mPum2. FIGS. 9B, D, and F illustrate data collected using a reporter assay similar to that which is described in FIG. 4 involving various tethering fusion proteins comprising mPum2. In summary, FIG. 9 illustrates that mPum2 fused to the double-stranded RNA binding domains of TRBP increases trans-splicing activity the most compared to other tethering fusion proteins and to a control trans-splicing RNA that lacks mPum2 binding sites. The figure legend describes whether a trans-splicing RNA is present (“+” indicates that a trans-splicing molecule with mPum2 sites is present, “+*” indicates a trans-splicing molecule lacking mPum2 sites is present, and “−” indicates that no trans-splicing molecule is present). The figure legend also describes the identities of the N- and C-terminal portions of the tethering fusion protein where parenthetical amino acid numbering indicates that a specific portion of the referenced protein is present.

FIGS. 10A-10B illustrate one example embodiment of the methods described herein. FIG. 10A illustrates a system composed of a donor RNA (e.g., a Replacement Domain encoding an exonic sequence that corresponds to a target RNA sequence or portion thereof) and an engineered small nuclear RNA (esnRNA). The combination of RNA donor molecule and esnRNA correct mutated RNAs via hybridization of the RNA donor to the target RNA carrying a mutation, followed by association of the esnRNA with the RNA donor, results in recruitment of spliceosome components and trans-splicing among the RNA donor molecule and the target RNA. This yields a corrected target RNA with the RNA donor molecule replacing a chosen sequence in the target RNA. FIG. 10B illustrates the how the components interact. Base pairing among the RNA donor and target RNA bring these molecule in close proximity. Base pairing among the esnRNA and the RNA donor brings spliceosome components in close proximity which promotes a trans-splicing reaction among the target RNA and the RNA donor.

FIG. 11 illustrates three example embodiments of the compositions and methods described in this disclosure. FIG. 11A describes a double trans-splicing molecule which carries two antisense domains, one replacement domain, two intronic domains, and at least two trans-splicing enhancer sequences within the intronic domains. This design promotes replacement of an internal sequence within the target RNA while maintaining the adjacent 5′ and 3′ sequences around the replaced sequence. FIG. 11B illustrates the design of a 3′ terminal trans-splicing RNA that will replace the 3′ terminal end of a target RNA while maintaining the 5′ end. FIG. 11C illustrates the design of a 5′ terminal trans-splicing molecule that will replace the 5′ terminal end of a target RNA while maintaining the 3′ end.

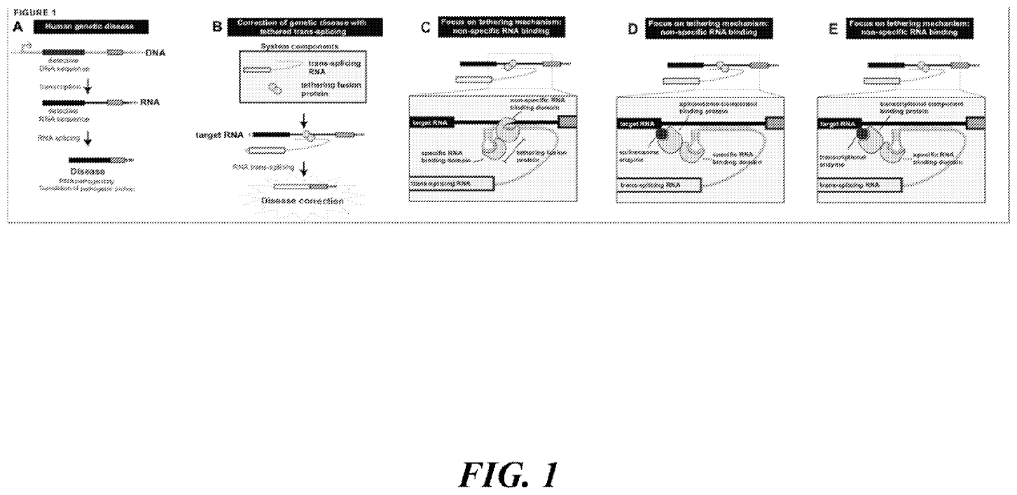

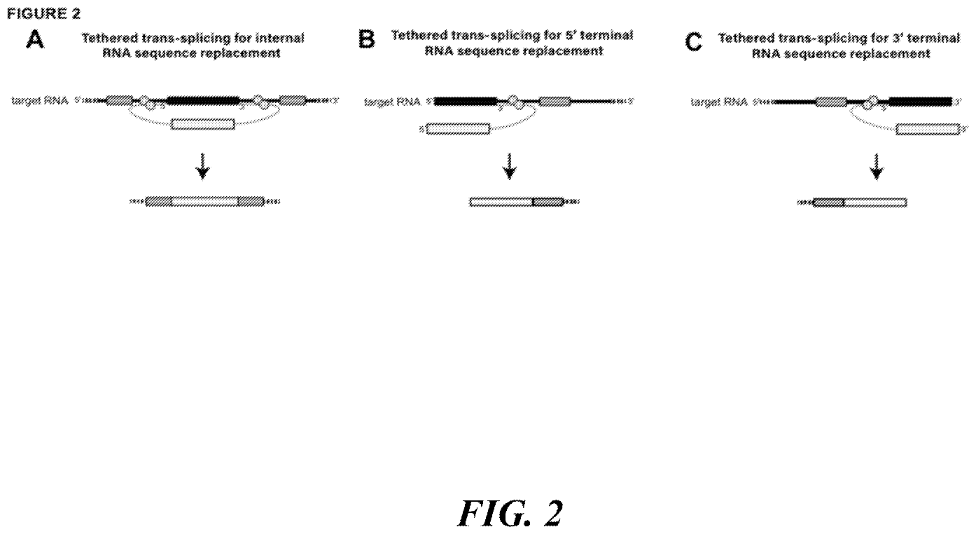

The patent or application file contains at least one drawing executed in color. Copies of this patent or patent application publication with color drawing(s) will be provided by the Office upon request and payment of the necessary fee.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides nucleic acids and proteins for the promotion of trans-splicing. A ribonucleic acid (RNA) sequence may have a mutation that, downstream, may lead to improper translation or processing of a polypeptide or protein. This may, in turn, cause any number of diseases. The present disclosure provides compositions and methods for treating or restoring a function of a target RNA sequence or portion thereof. Compositions and methods as disclosed herein may promote the association of a trans-splicing RNA to the target RNA. The trans-splicing RNA may comprise a nucleic acid molecule encoding one or more binding domains. The one or more binding domains may be configured to interact with an RNA-binding protein. The nucleic acid molecule may encode one or more exonic domains. The nucleic acid molecule may comprise RNA or deoxyribonucleic acid (DNA). The nucleic acid molecule may be transcribed from DNA into RNA. For example, a DNA molecule encoding one or more exonic domains and/or one or more binding domains may be transcribed into an RNA molecule comprising one or more exonic domains and/or one or more binding domains. The one or more exonic domains may correspond to a target ribonucleic acid (RNA) sequence of portion thereof. The one or more exonic domains may bind to or replace the target RNA sequence or portion thereof. The target RNA sequence or portion thereof may have a mutated or missing sequencing. The binding or replacing of the target RNA sequence or portion thereof with the one or more exonic domains may treat or restore a function of the target RNA sequence or portion thereof. The RNA binding protein may be configured to insert the exonic sequence into the target RNA sequence or portion thereof. The RNA-binding protein may be encoded by one or more human-derived sequences.

The trans-splicing nucleic acid may be provided in a cell. The cell may be a human cell. For example, the DNA molecule or the RNA molecule may be provided in a human cell. The target RNA may be in a cell. For example, the target RNA may be in a human cell. The target RNA may be a messenger RNA (mRNA). The trans-splicing molecule may encode an intronic domain. For example, a trans-splicing RNA molecule may encode an intronic domain. For example, a trans-splicing DNA molecule may encode an intronic domain. The trans-splicing nucleic acid may encode a replacement domain comprising one or more exonic sequences. For example, a trans-splicing RNA or DNA molecule may encode a replacement domain comprising one or more exonic sequences The replacement domain may comprise a gene that encodes a protein, or a portion thereof. The trans-splicing nucleic acid may comprise one or more intronic domains. The trans-splicing nucleic acid may comprise one or more replacement domains. The replacement domain may nucleic acid one or more genes. The one or more genes may encode one or more proteins. The trans-splicing nucleic acid may comprise one or more binding domains configured to interact with an RNA-binding protein. The RNA-binding protein may be a tethering protein. A tethering protein may associate the trans-splicing nucleic acid with the target RNA. The tethering protein may be a fusion protein. The tethering protein may comprise a domain that associates with a spliceosome. The tethering protein may comprise a domain that associates with a transcriptional enzyme. The tethering protein may comprise a domain that binds double-stranded RNA non-specifically. Provided herein are compositions that bring trans-splicing nucleic acids and their target RNAs in close proximity in the cellular nucleus to increase the efficiency of RNA editing by the trans-splicing RNA. Provided herein are methods for replacement of chosen RNA sequences within target RNAs using RNA trans-splicing molecules to treat a disease in the context of a human gene therapy.

### Compositions

The present disclosure provides compositions comprising nucleic acids and proteins for trans-splicing. The composition may comprise a nucleic acid encoding an exonic sequence correspondence to a sequence or a portion thereof in a target RNA. The sequence of the target RNA may comprise a missing or mutated sequence. The trans-splicing of the exonic sequence to the target RNA may correct the missing or mutated sequence. The nucleic acid may further comprise an intronic domain comprising a sequence to enhance the trans-splicing. The composition may further comprise a protein. The protein may be a fusion protein. The protein may promote an association of the exonic sequence to the target RNA. The nucleic acid may comprise deoxyribonucleic acid (DNA). The nucleic acid comprising DNA may be reverse-transcribed into a trans-splicing RNA molecule. The nucleic acid may comprise ribonucleic acid (RNA).

### Nucleic Acid

The present disclosure provides a nucleic acid encoding an exonic sequence. The nucleic acid may comprise DNA. The nucleic acid comprising DNA may be transcribed into RNA, e.g., a trans-splicing RNA molecule comprising the exonic sequence. The exonic sequence may correspond to a sequence or portion thereof of a target RNA. The exonic sequence may associate with the target RNA. The exonic sequence may be trans-spliced into the sequence of the target RNA. The sequence of the target RNA may be mutated or missing a sequence. The trans-splicing of the exonic sequence to the target RNA may correct the missing or mutated sequence of the target RNA. The nucleic acid may comprise RNA, e.g., the nucleic acid may be a trans-splicing RNA molecule. In some embodiments, the trans-spicing ribonucleic acid molecule comprises a replacement domain, an intronic domain, and at least one binding site for the tethering protein. In some embodiments, the trans-splicing RNA molecule comprises: (a) at least one domain that promotes trans-splicing (“Intronic Domain”), (b) at least one binding domain (“Antisense Domain”) that comprises or consists of a sequence complementary to a pre-mRNA present in a human cells (“Target RNA”), (c) a coding domain that is inserted into the Target RNA via trans-splicing (“Replacement Domain”), and (d) at least one binding site for the tethering protein.

In some embodiments, the trans-splicing RNA comprises a single binding site for the specific RNA binding domain. In some embodiments, the trans-splicing RNA comprises two or more binding sites for the specific RNA binding domain. In some embodiments, the trans-splicing RNA comprises one or more binding domains. The one or more binding domains may be configured to interact with one or more RNA-binding proteins. The RNA-binding proteins may be encoded by one or more human-derived sequences. The one or more RNA-binding proteins may be encoded by one or more non-human-derived sequences. In some embodiments, the binding sites for the specific RNA binding domain is isolated or derived from the sequence of the SLBP binding site. In some embodiments, the binding sites for the specific RNA binding domain is isolated or derived from a sequence that is targeted by a PUF or Pumby protein. In some embodiments, the SLBP binding site comprises or consist of the following sequence: CCAAAGGCTCTTCTCAGAGCCACCCA (SEQ ID NO: 1). In some embodiments, the SLBP binding site comprises at least about 60%, about 65%, about 70%, about 75%, about 80%, about 85%, about 90%, about 95%, about 97.5%, about 98%, about 99%, or about 100% identity with a sequence encoded by SEQ ID NO: 1.

In some embodiments, the trans-splicing nucleic acid is RNA, DNA, a DNA/RNA hybrid, and/or comprises at least one of a nucleic acid analog, a chemically-modified nucleic acid, or a chimera composed of two or more nucleic acids or nucleic acid analogs. As used herein, the term “nucleic acid analog” refers to a compound having structural similarity to a canonical purine or pyrimidine base occurring in DNA or RNA. The nucleic acid analog may comprise a modified sugar and/or a modified nucleobase, as compared to a purine or pyrimidine base occurring naturally in DNA or RNA. In some embodiments, the nucleic acid analog is a 2′-deoxyribonucleoside, 2′-ribonucleoside, 2′-deoxyribonucleotide or a 2′-ribonucleotide, wherein the nucleobase includes a modified base (such as, for example, xanthine, uridine, oxanine (oxanosine), 7-methlguanosine, dihydrouridine, 5-methylcytidine, C3 spacer, 5-methyl dC, 5-hydroxybutynl-2′-deoxyuridine, 5-nitroindole, 5-methyl iso-deoxycytosine, iso deoxyguanosine, deoxyuradine, iso deoxycytidine, other 0-1 purine analogs, N-6-hydroxylaminopurine, nebularine, 7-deaza hypoxanthine, other 7-deazapurines, and 2-methyl purines). In some embodiments, the nucleic acid analog may be selected from the group consisting of inosine, 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In other embodiments the nucleic acid analog is a nucleic acid mimic (such as, for example, artificial nucleic acids and xeno nucleic acids (XNA).

### Intronic Domain

The present disclosure provides nucleic acids encoding an Intronic Domain. In some embodiments, the nucleic acid encodes one or more Intronic Domains. In some embodiments, the nucleic acid comprises a DNA. In some embodiments, the nucleic acid comprises an RNA.

In some embodiments, the Intronic Domains carry binding sites that are targeted by RNA-binding proteins with disease-causing mutations. In some embodiments, the dissociation constant of these mutated RNA-binding proteins and the Intronic Domain is lower than the dissociation constant of the non-mutated RNA-binding protein and the Intronic Domain.

In some embodiments, the intronic domain is configured to promote insertion of a replacement domain into a target mRNA molecule. In some embodiments, the intronic domain comprises one or more binding sites configured to interact with an RNA binding protein. In some embodiments, the RNA binding protein is a tethering protein. In some embodiments, the RNA binding protein is encoded by one or more human-derived sequences. In some embodiments, the tethering protein is a tethering protein.

### Antisense Domains

The present disclosure provides a nucleic acid encoding an exonic sequence that may correspond to a sequence or portion thereof of a target RNA. In some embodiments of the compositions of the disclosure, a target RNA comprises a pathogenic RNA. In some embodiments, the target RNA comprises a target sequence that is complementary to an antisense domain encoded by the nucleic acid.

In some embodiments of the compositions and methods of the disclosure, the target sequence comprises or consists of between 5 and 500 nucleotides. In some embodiments, the target sequence comprises or consists of between 50 and 250 nucleotides. In some embodiments, the target sequence comprises or consists of between 5 and 50 nucleotides.

In some embodiments of the compositions and methods of the disclosure, a target sequence is comprised within a single contiguous stretch of the target RNA. In some embodiments, the target sequence may consist of comprise of one or more nucleotides that are not spread among a single contiguous stretch of the target RNA.

In some embodiments of the disclosure, an Antisense Domain of the disclosure binds to a target sequence. In some embodiments of the disclosure, an antisense domain of the disclosure binds to a target RNA.

In some embodiments of the disclosure, the Antisense Domain is chosen so that successful trans-splicing causes removal of micro open reading frames in the target RNA. In this manner, the trans-splicing system removes micro open reading frames and increases the production of protein from the target RNA.

In some embodiments of the compositions of the disclosure, the sequence comprising the Antisense Domain has at least about 50%, 55%, 60%, 65%, 70%, 75%, 80%, 87%, 90%, 91%, 92%, 93%, 94%, 95%, 96%, 97%, 98%, 99% or any percentage in between of complementarity to the target RNA sequence. In some embodiments, the Antisense Domain has 100% complementarity to the Target RNA sequence. In some embodiments, the Antisense Domain comprises or consists of about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 210 nucleotides, about 220 nucleotides, about 230 nucleotides, 240 nucleotides, about 250 nucleotides, 260 nucleotides, about 270 nucleotides, about 270 nucleotides, or more complementary to the Target RNA sequence.

In some embodiments, the Antisense Domain is complementary to an RNA transcribed from a gene that is selected from the group consisting of: TNFRSF13B [ENSG00000240505](common variable immune deficiency); ADA, CECR1 [ENSG00000196839, ENSG00000093072](Adenosine deaminase deficiency); IL2RG [ENSG00000147168](X-linked severe combined immunodeficiency); HBB [ENSG00000244734](Beta-thassalemia); HBA1, HBA2 [ENSG00000206172, ENSG00000188536](alpha-thassalemia); U2AF1 [ENSG00000160201](myelodysplastic syndrome); SOD1, TARDBP, FUS, MATR3, SOD1, C90RF72 [ENSG00000142168, ENSG00000120948, ENSG00000089280, ENSG00000015479, ENSG00000142168, ENSG00000147894](Amyotrophic lateral sclerosis); MAPT, PGRN [ENSG00000186868, ENSG00000030582](Frontotemporal dementia with parkinsonism); CDH23, MYO7A, USH2A [ENSG00000107736, ENSG00000137474, ENSG00000042781](Usher's syndrome); GALC [ENSG00000054983](Krabbe disease); SMPD1, NPC1, NPC2 [ENSG00000166311, ENSG00000141458, ENSG00000119655](Niemann Pick disease); PRNP [ENSG00000171867](prion disease); SCN1A [ENSG00000144285](Dravet syndrome); PINK1, ATPGAP2 [ENSG00000158828](early-onset Parkinson's disease); ATXN1, ATXN2, ATXN3, PLEKHG4, SPTBN2, CACNA1A, ATXN7, TTBK2, PPP2R2B, KCNC3, PRKCG, ITPR1, TBP, KCND1, FGF14 [ENSG00000124788, ENSG00000204842, ENSG00000066427, ENSG00000196155, ENSG00000173898, ENSG00000141837, ENSG00000163635, ENSG00000128881, ENSG00000156475, ENSG00000131398, ENSG00000126583, ENSG00000150995, ENSG00000112592, ENSG00000102057, ENSG00000102466](spinocerebellar ataxias); SCN1A, SCN2A, CACNA1A, GRIN2B, GRIN2A, MECP2, FOXG1, SLC6A1, PRRT2, PTEN, KCNQ2, KCNQ3, STARD7, CLRN1 [ENSG00000144285, ENSG00000136531, ENSG00000141837, ENSG00000273079, ENSG00000183454, ENSG00000169057, ENSG00000176165, ENSG00000157103, ENSG00000167371, ENSG00000171862, ENSG00000075043, ENSG00000184156, ENSG00000084090, ENSG00000163646](genetic epilepsy disorders); ATM [ENSG00000149311](Ataxia-telangiectasia); GLB1 [ENSG00000170266](GM1 gangliosidosis); GBA [ENSG00000177628](Gaucher disease); GM2A [ENSG00000196743](GM2 gangliosidosis); UBE3A [ENSG00000114062](Angelman syndrome); SLC2A1 [ENSG00000117394](glucose transporter deficiency type 1); LAMP2 [ENSG00000005893](Danon disease); GLA [ENSG00000102393](Fabry disease); PKD1, PKD2 [ENSG00000008710, ENSG00000118762](Autosomal dominant polycystic kidney disease); GAA [ENSG00000171298](Pompe disease); PCSK9, LDLR, APOB, APOE [ENSG00000169174, ENSG00000130164, ENSG00000084674, ENSG00000130203](Familial hypercholesterolemia); MYOC, OPTN, TBK1, WDR36, CYPIB1 [ENSG00000034971, ENSG00000123240, ENSG00000183735, ENSG00000134987, ENSG00000138061](Open Angle Glaucoma); IDUA [ENSG00000127415](Hurler syndrome or Mucopolysaccharidosis 1); IDS [ENSG00000010404](Hunter syndrome or Mucopolysaccharidosis 2); CLN3 [ENSG00000188603](Batten disease); DMD [ENSG00000198947](Duchenne muscular dystrophy); LMNA [ENSG00000160789](Limb-girdle muscular dystrophy type 1B); DYSF [ENSG00000135636](Limb-girdle muscular dystrophy type 2B); SGCA [ENSG00000108823](Limb-girdle muscular dystrophy type 2D); SGCB [ENSG00000163069](Limb-girdle muscular dystrophy type 2E); SGCG [ENSG00000102683](Limb-girdle muscular dystrophy type 2C); SGCD [ENSG00000170624](Limb-girdle muscular dystrophy type 2F); DUX4 [ENSG00000260596](Facioscapulohumeral muscular dystrophy); F9 [ENSG00000101981](Hemophilia B); F8 [ENSG00000185010](Hemophilia A); USHA2A, RPGR, RP2, RHO, PRPF31, USH1F, PRPF3, PRPF6 [ENSG00000156313, ENSG00000102218, ENSG00000163914, ENSG00000105618, ENSG00000150275, ENSG00000117360, ENSG00000101161](Retinitis pigmentosa); CFTR [ENSG00000001626](cystic fibrosis); GJB2, GJB6, STRC, DFNA1, WFS1 [ENSG00000165474, ENSG00000121742, ENSG00000242866, ENSG00000131504, ENSG00000109501](autosomal dominant hearing impairment); POU3F3 [ENSG00000198914](nonsyndromic hearing loss).

### Replacement Domains

In some embodiments, described herein is a nucleic acid encoding a Replacement Domain. In some embodiments, the Replacement domain is derived or isolated from the Target RNA. The Replacement Domain may encode an exonic sequence corresponding to a sequence or portion thereof of a target RNA. A trans-splicing of the Replacement Domain to the target RNA sequence or portion thereof may correct a missing or mutated sequence of the target RNA.

In some embodiments, the Replacement Domain encodes a sequence derived or isolated from a human gene. In some embodiments of the compositions of the disclosure, the sequence encoding the Replacement Domain has at least about 50%, 55%, 60%, 65%, 70%, 75%, 80%, 87%, 90%, 95%, 97%, 99% or any percentage in between of identity with a human gene. In some embodiments, the Replacement Domain has about 100% identity with a sequence derived or isolated from a human gene. In some embodiments, the Replacement Domain comprises or consists of about 2 nucleotides, about 5 nucleotides, about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, about 200 nucleotides, about 210 nucleotides, about 220 nucleotides, about 230 nucleotides, about 240 nucleotides, about 250 nucleotides, about 260 nucleotides, about 270 nucleotides, about 270 nucleotides, or more.

Compositions comprising replacement domains disclosed herein include any strategies where replacement or insertion of RNA sequences can be an effective therapy. Non-limiting examples of replacement domains include sequences derived or isolated from the following genes (with gene accession IDs in brackets and associated diseases in parentheses) such as TNFRSF13B [ENSG00000240505](common variable immune deficiency); ADA, CECR1 [ENSG00000196839, ENSG00000093072](Adenosine deaminase deficiency); IL2RG [ENSG00000147168](X-linked severe combined immunodeficiency); HBB [ENSG00000244734](Beta-thassalemia); HBA1, HBA2 [ENSG00000206172, ENSG00000188536](alpha-thassalemia); U2AF1 [ENSG00000160201](myelodysplastic syndrome); SOD1, TARDBP, FUS, MATR3, SOD1, C90RF72 [ENSG00000142168, ENSG00000120948, ENSG00000089280, ENSG00000015479, ENSG00000142168, ENSG00000147894](Amyotrophic lateral sclerosis); MAPT, PGRN [ENSG00000186868, ENSG00000030582](Frontotemporal dementia with parkinsonism); CDH23, MYO7A, USH2A [ENSG00000107736, ENSG00000137474, ENSG00000042781](Usher's syndrome); GALC [ENSG00000054983](Krabbe disease); SMPD1, NPC1, NPC2 [ENSG00000166311, ENSG00000141458, ENSG00000119655](Niemann Pick disease); PRNP [ENSG00000171867](prion disease); SCN1A [ENSG00000144285](Dravet syndrome); PINK1, ATPGAP2 [ENSG00000158828](early-onset Parkinson's disease); ATXN1, ATXN2, ATXN3, PLEKHG4, SPTBN2, CACNA1A, ATXN7, TTBK2, PPP2R2B, KCNC3, PRKCG, ITPR1, TBP, KCND1, FGF14 [ENSG00000124788, ENSG00000204842, ENSG00000066427, ENSG00000196155, ENSG00000173898, ENSG00000141837, ENSG00000163635, ENSG00000128881, ENSG00000156475, ENSG00000131398, ENSG00000126583, ENSG00000150995, ENSG00000112592, ENSG00000102057, ENSG00000102466](spinocerebellar ataxias); SCN1A, SCN2A, CACNA1A, GRIN2B, GRIN2A, MECP2, FOXG1, SLC6A1, PRRT2, PTEN, KCNQ2, KCNQ3, STARD7, CLRN1 [ENSG00000144285, ENSG00000136531, ENSG00000141837, ENSG00000273079, ENSG00000183454, ENSG00000169057, ENSG00000176165, ENSG00000157103, ENSG00000167371, ENSG00000171862, ENSG00000075043, ENSG00000184156, ENSG00000084090, ENSG00000163646](genetic epilepsy disorders); ATM [ENSG00000149311](Ataxia-telangiectasia); GLB1 [ENSG00000170266](GM1 gangliosidosis); GBA [ENSG00000177628](Gaucher disease); GM2A [ENSG00000196743](GM2 gangliosidosis); UBE3A [ENSG00000114062](Angelman syndrome); SLC2A1 [ENSG00000117394](glucose transporter deficiency type 1); LAMP2 [ENSG00000005893](Danon disease); GLA [ENSG00000102393](Fabry disease); PKD1, PKD2 [ENSG00000008710, ENSG00000118762](Autosomal dominant polycystic kidney disease); GAA [ENSG00000171298](Pompe disease); PCSK9, LDLR, APOB, APOE [ENSG00000169174, ENSG00000130164, ENSG00000084674, ENSG00000130203](Familial hypercholesterolemia); MYOC, OPTN, TBK1, WDR36, CYPIB1 [ENSG00000034971, ENSG00000123240, ENSG00000183735, ENSG00000134987, ENSG00000138061](Open Angle Glaucoma); IDUA [ENSG00000127415](Hurler syndrome or Mucopolysaccharidosis 1); IDS [ENSG00000010404](Hunter syndrome or Mucopolysaccharidosis 2); CLN3 [ENSG00000188603](Batten disease); DMD [ENSG00000198947](Duchenne muscular dystrophy); LMNA [ENSG00000160789](Limb-girdle muscular dystrophy type 1B); DYSF [ENSG00000135636](Limb-girdle muscular dystrophy type 2B); SGCA [ENSG00000108823](Limb-girdle muscular dystrophy type 2D); SGCB [ENSG00000163069](Limb-girdle muscular dystrophy type 2E); SGCG [ENSG00000102683](Limb-girdle muscular dystrophy type 2C); SGCD [ENSG00000170624](Limb-girdle muscular dystrophy type 2F); DUX4 [ENSG00000260596](Facioscapulohumeral muscular dystrophy); F9 [ENSG00000101981](Hemophilia B); F8 [ENSG00000185010](Hemophilia A); USHA2A, RPGR, RP2, RHO, PRPF31, USH1F, PRPF3, PRPF6 [ENSG00000156313, ENSG00000102218, ENSG00000163914, ENSG00000105618, ENSG00000150275, ENSG00000117360, ENSG00000101161](Retinitis pigmentosa); CFTR [ENSG00000001626](cystic fibrosis); GJB2, GJB6, STRC, DFNA1, WFS1 [ENSG00000165474, ENSG00000121742, ENSG00000242866, ENSG00000131504, ENSG00000109501](autosomal dominant hearing impairment); POU3F3 [ENSG00000198914](nonsyndromic hearing loss).

In some embodiments, the replacement domain is codon optimized. In some embodiments, the replacement sequence is codon optimized in a manner that increases the stability, translation, or other desirable features.

In addition to sequences derived from human genes, Replacement Domains can comprise sequences derived from other organisms in order to alter the stability, translation, processing, or localization of a target RNA. Non-limiting examples of replacement domains derived from non-human sources include sequences that increase protein production such as those derived or isolated from Woodchuck Hepatitis Virus (WHV) Post-transcriptional Regulatory Element (WPRE), triplex from MALAT1, the PRE of Hepatitis B virus (HPRE), and an iron response element of the form CAGYCX (Y=U or A; X=U, C, or A).

### Localization Domain

The present disclosure provides nucleic acids encoding a localization sequence. A localization sequence may comprise one or more sequences, e.g., nuclear localization sequence, that may promote the accumulation of compositions as described herein in a cellular nucleus. In eukaryotes, the process of transcription takes place in a cellular nucleus. To that end, an increased accumulation of nucleic acids for trans-splicing to the nucleus may increase the occurrence of trans-splicing.

Compositions as described herein may comprise a nucleic acid encoding a localization sequence. The nucleic acid may comprise RNA. The RNA encoding the localization sequence may further encode an exonic sequence corresponding to a target RNA. The localization sequence on the RNA may promote trans-splicing of the exonic sequence into the target RNA. The nucleic acid may comprise DNA encoding a localization sequence. The DNA encoding the localization sequence may be transcribed into RNA. The DNA may further encode an exonic sequence corresponding to a target RNA. The DNA encoding the exonic sequence may be transcribed into RNA. In this manner, a DNA molecule encoding the localization sequence and the exonic sequence may be transcribed into RNA, and the localization sequence on the RNA may promote trans-splicing of the exonic sequence into the target RNA. The trans-splicing of the exonic sequence into the RNA may treat, e.g., a mutation of the target RNA. A variety of RNA sequences placed in a heterologous context may promote the accumulation of RNAs in the nucleus or within specific structures in the nucleus such as nuclear speckles or paraspeckles. The present disclosure further assesses 1) whether the presence of localization sequences interferes with trans-splicing reactions, 2) which putative localization sequences function in the context of trans-splicing, and 3) whether the accumulation of trans-splicing molecules in specific locations increases RNA trans-splicing efficiency. As the activity of many known RNA localization sequences may be context-dependent, the present disclosure provides a distinct group of localization sequences that may function in the context of trans-splicing. This is confirmed by experiments that indicate that activity of localization in other contexts (i.e., outside of the scope of trans-splicing) is not necessarily predictive of activity in trans-splicing.

In some instances, a trans-splicing molecule provided herein can comprise localization sequences. In some instances, a trans-splicing molecule provided herein may not comprise localization sequences. In some embodiments, localization sequences that increase trans-splicing activity can also increase the levels of trans-splicing molecule. In some embodiments, a localization sequence described herein can be derived from mRNA, long noncoding RNAs, and synthetic sequences that can alter that localization of varied transcript types within the cellular nucleus. In some embodiments, a localization sequence described herein can function specifically within the context of trans-splicing. In some embodiments, a localization sequence described herein can function universally (e.g., any systems)

The Localization Domain may promote transport of the trans-splicing nucleic acid to the cellular nucleus or to specific locations within the cellular nucleus. The Localization Domain may comprise one or more localization sequences that bind to enzymes involved in transcription (such as polymerase II or transcription-associated enzymes), RNA splicing, or the formation of nuclear speckles. There exist various means to promote RNA trans-splicing and the present disclosure focuses on RNA trans-splicing that is mediated by the cellular spliceosome. As the components on the spliceosome may be located inside and within the cellular nucleus, the Localization Domain may increase RNA trans-splicing activity by promoting accumulation of the RNA trans-splicing molecule to the location of the spliceosome. In other embodiments, the present disclosure provides a composition comprising a nucleic acid sequence encoding the trans-splicing nucleic acid molecule.

In some embodiments, the Localization Domain binds to polymerase II and is derived or isolated from an aptamer or long noncoding RNA. In some embodiments, the Localization Domain carries sequences that promotes nuclear localization of the trans-splicing molecule. In some embodiments, the Localization Domain is derived from a long noncoding RNA.

In some embodiments, the Localization Domain is derived or isolated from a short interspersed element (SINE). In some embodiments, the Localization Domain binds to proteins involved in transcription. In some embodiments, the Localization Domain binds to proteins involved in RNA splicing.

In some embodiments, the Localization Domain promotes accumulation of the trans-splicing molecule in nuclear paraspeckles. In some embodiments, the Localization Domain that promotes accumulation of the trans-splicing molecule in nuclear paraspeckles is derived or isolated from a gene selected from the group consisting of: lnc-LTBP3-10 [lnc-LTBP3-10], SLC29A2 [ENSG00000174669.12], SNHG1 [ENSG00000255717.7], MUS81 [ENSG00000172732.12], TCIRG1 [ENSG00000110719.10], INPPL1 [ENSG00000165458.14], lnc-ANAPC11-7 [lnc-ANAPC11-7], IL18BP [ENSG00000137496.18], POLA2 [ENSG00000014138.9], PCNX3 [ENSG00000197136.4], PC [ENSG00000173599.15], RBM4 [ENSG00000173933.20], lnc-KCNK7-6 [lnc-KCNK7-6], EML3 [ENSG00000149499.11], PGGHG [ENSG00000142102.16], RBM14 [ENSG00000239306.4], LTBP3 [ENSG00000168056.16], ATG2A [ENSG00000110046.13], XLOC_026224 [XLOC_026224], HERC2P2 [ENSG00000276550.4], WDR90 [ENSG00000161996.19], lnc-LTBP3-2 [lnc-LTBP3-2], LENG8 [ENSG00000167615.16], TPCN2 [ENSG00000162341.18], lnc-TCIRG1-1 [lnc-TCIRG1-1], ATG16L2 [ENSG00000168010.11], MROH1 [ENSG00000179832.17], CCDC57 [ENSG00000176155.19], lnc-LTBP3-11 [lnc-LTBP3-11], PIDD1 [ENSG00000177595.18], lnc-VSTM5-1 [lnc-VSTM5-1], NEAT1 [ENSG00000245532.9], XLOC_079850 [XLOC_079850], XLOC_028656 [XLOC_028656], DNHD1 [ENSG00000179532.12], ABCA7 [ENSG00000064687.12], XLOC_000636 [XLOC_000636], MAN2C1 [ENSG00000140400.17], lnc-SSH3-5 [lnc-SSH3-5], MIRLET7BHG [ENSG00000197182.14], MAMDC4 [ENSG00000177943.14], NAA40 [ENSG00000110583.13], ANKRD13D [ENSG00000172932.14], lnc-NUMA1-3 [lnc-NUMA1-3], ADAMTS10 [ENSG00000142303.14], XLOC_083799 [XLOC_083799], ARHGEF17 [ENSG00000110237.5], CDC42BPG [ENSG00000171219.9], SNAPC4 [ENSG00000165684.4], lnc-CFL1-1 [lnc-CFL1-1], B4GALNT4 [ENSG00000182272.12], XLOC_027567 [XLOC_027567], XLOC_000644 [XLOC_000644], XLOC_024022 [XLOC_024022], LTO1 [ENSG00000149716.12], AC064843.1 [ENSG00000286621.1], CHRND [ENSG00000135902.10], ASPSCR1 [ENSG00000169696.16], RAD9A [ENSG00000172613.8], lnc-RTN4R-1 [lnc-RTN4R-1], lnc-MRPL11-1 [lnc-MRPL11-1], SSH3 [ENSG00000172830.13], XLOC_000637 [XLOC_000637], AP000873.2 [ENSG00000247137.9], lnc-TRPT1-4 [lnc-TRPT1-4], XLOC_027568 [XLOC_027568], LINC01503 [ENSG00000233901.6], RNASEH2C [ENSG00000172922.9], XLOC_000634 [XLOC_000634], MYO7A [ENSG00000137474.22], XLOC_000633 [XLOC_000633], lnc-BCL3-1 [lnc-BCL3-1], MTMR9LP [ENSG00000220785.7], AP5B1 [ENSG00000254470.3], lnc-EDF1-2 [lnc-EDF1-2], lnc-UNC93B1-1 [lnc-UNC93B1-1], GOLGA8B [ENSG00000215252.11], MSH5 [ENSG00000204410.15], AP003119.1 [ENSG00000254632.2], GUSBP11 [ENSG00000228315.12], RPS6KB2 [ENSG00000175634.15], EME2 [ENSG00000197774.13], XLOC_028057 [XLOC_028057], FRMD8 [ENSG00000126391.14], lnc-OGFOD3-1 [lnc-OGFOD3-1], XLOC_152482 [XLOC_152482], XLOC_028434 [XLOC_028434], ZNF276 [ENSG00000158805.12], AP000944.5 [ENSG00000285816.1], NRBP2 [ENSG00000185189.18], NDOR1 [ENSG00000188566.13], lnc-PHYHD1-1 [lnc-PHYHD1-1], lnc-RECQL4-3 [lnc-RECQL4-3], lnc-UAP1L1-4 [lnc-UAP1L1-4], MSH5-SAPCD1 [ENSG00000255152.8], lnc-P2RY6-1 [lnc-P2RY6-1], RELT [ENSG00000054967.13], CPNE7 [ENSG00000178773.15], XLOC_028557 [XLOC_028557], XLOC_156663 [XLOC_156663], CORO6 [ENSG00000167549.18], RTEL1 [ENSG00000258366.8], MIR34AHG [ENSG00000228526.7], STPG3-AS1 [ENSG00000275549.1], lnc-WFIKKN2-4 [lnc-WFIKKN2-4], SYNGAP1 [ENSG00000197283.17], LRRC45 [ENSG00000169683.8], KIAA0895L [ENSG00000196123.13], PNKP [ENSG00000039650.12], lnc-EIF1AD-5 [lnc-EIF1AD-5], TM7SF2 [ENSG00000149809.15], NSUN5P2 [ENSG00000106133.18], lnc-POLR2L-1 [lnc-POLR2L-1], lnc-PPP1R27-1 [lnc-PPP1R27-1], AC110285.2 [ENSG00000262877.5], lnc-LRRC32-5 [lnc-LRRC32-5], AC131009.4 [ENSG00000279283.1], BBS1 [ENSG00000174483.20], XLOC_061408 [XLOC_061408], lnc-SERPINH1-3 [lnc-SERPINH1-3], AC027601.6 [ENSG00000287431.1], lnc-NFAM1-3 [lnc-NFAM1-3], EXD3 [ENSG00000187609.16], AC009022.1 [ENSG00000196696.12], MC1R [ENSG00000258839.3], PKD1P6 [ENSG00000250251.6], lnc-KLHL35-6 [lnc-KLHL35-6], Z97832.2 [ENSG00000272374.1], C19orf25 [ENSG00000119559.16], lnc-TMEM138-3 [lnc-TMEM138-3], ALO31595.3 [ENSG00000280434.1], lnc-LRRC56-3 [lnc-LRRC56-3], lnc-STIP1-2 [lnc-STIP1-2], XLOC_095699 [XLOC_095699], SSSCA1-AS1 [ENSG00000260233.3], NPDC1 [ENSG00000107281.10], lnc-NR1D1-1 [lnc-NR1D1-1], lnc-RPL12-1 [lnc-RPL12-1], lnc-MRPL49-1 [lnc-MRPL49-1], XLOC_061398 [XLOC_061398], TOB1-AS1 [ENSG00000229980.5], AC127502.1 [ENSG00000215302.8], XLOC_149046 [XLOC_149046], lnc-TRMT112-4 [lnc-TRMT112-4], LINC02593 [ENSG00000223764.2], KLHL17 [ENSG00000187961.14], lnc-KLHL35-7 [lnc-KLHL35-7], lnc-TMEM258-2 [lnc-TMEM258-2], AP002495.1 [ENSG00000254469.7], XLOC_024025 [XLOC_024025], GPSM1 [ENSG00000160360.13], XLOC_152839 [XLOC_152839], LBHD1 [ENSG00000162194.12], GATD1 [ENSG00000177225.17], XLOC_149045 [XLOC_149045], LENG8-AS1 [ENSG00000226696.6], MAP4K2 [ENSG00000168067.12], C11orf80 [ENSG00000173715.16], MAPK8IP3 [ENSG00000138834.12], XLOC_090526 [XLOC_090526], KIFC2 [ENSG00000167702.12], LRP5L [ENSG00000100068.13], SEC31B [ENSG00000075826.17], XLOC_024171 [XLOC_024171], PPP2R5B [ENSG00000068971.14], lnc-GIPC3-3 [lnc-GIPC3-3], AC020916.1 [ENSG00000267519.6], XLOC_156901 [XLOC_156901], AP006333.1 [ENSG00000256341.1], lnc-ZNF778-3 [lnc-ZNF778-3], lnc-LAMA5-1 [lnc-LAMA5-1], lnc-TMEM106A-3 [lnc-TMEM106A-3], lnc-ACER3-1 [lnc-ACER3-1], RHPN1 [ENSG00000158106.14], XLOC_028558 [XLOC_028558], XLOC_088401 [XLOC_088401], BX255925.3 [ENSG00000284976.1], GUCY2EP [ENSG00000204529.4], XLOC_152506 [XLOC_152506], NOXA1 [ENSG00000188747.8], lnc-ARRDC1-2 [lnc-ARRDC1-2], XLOC_145191 [XLOC_145191], BSCL2 [ENSG00000168000.14], lnc-MACROD1-1 [lnc-MACROD1-1], AL162586.1 [ENSG00000225032.5], AP000944.7 [ENSG00000287917.1], AC091196.1 [ENSG00000285581.1], ZNRD2 [ENSG00000173465.8], XLOC_026268 [XLOC_026268], OSBPL7 [ENSG00000006025.12], lnc-SSH3-4 [lnc-SSH3-4], C9orf106 [ENSG00000179082.3], AP000437.1 [ENSG00000279549.1], lnc-NCOA3-14 [lnc-NCOA3-14], NADSYN1 [ENSG00000172890.13], XLOC_060204 [XLOC_060204], lnc-SHANK2-1 [lnc-SHANK2-1], MEGF6 [ENSG00000162591.16], AC099811.1 [ENSG00000236194.3], ME3 [ENSG00000151376.16], XLOC_028655 [XLOC_028655], GDPD5 [ENSG00000158555.15], lnc-SPDYC-2 [lnc-SPDYC-2], AC0008105.3 [ENSG00000267121.6], lnc-NCOA3-21 [lnc-NCOA3-21], lnc-FEN1-6 [lnc-FEN1-6], lnc-HYOU1-1 [lnc-HYOU1-1], AC102953.2 [ENSG00000273230.1], XLOC_095073 [XLOC_095073], LINC00235 [ENSG00000277142.1], AL355987.4 [ENSG00000273066.5], XLOC_152404 [XLOC_152404], lnc-CDK12-1 [lnc-CDK12-1], XLOC_028004 [XLOC_028004], lnc-CCDC154-2 [lnc-CCDC154-2], lnc-CCDC87-1 [lnc-CCDC87-1], INPP5E [ENSG00000148384.13], XLOC_021222 [XLOC_021222], AJM1 [ENSG00000232434.2], HSF4 [ENSG00000102878.16], LINC00313 [ENSG00000185186.10], lnc-UNC93B1-7 [lnc-UNC93B1-7], lnc-PIDD1-2 [lnc-PIDD1-2], lnc-CSNK1G2-5 [lnc-CSNK1G2-5], lnc-UNC93B1-5 [lnc-UNC93B1-5], AP006621.3 [ENSG00000255284.2], CCDC78 [ENSG00000162004.17], lnc-HAAO-7 [lnc-HAAO-7], EFEMP2 [ENSG00000172638.13], XLOC_000635 [XLOC_000635], XLOC_147952 [XLOC_147952], lnc-PKNOX1-1 [lnc-PKNOX1-1], lnc-LTBP3-9 [lnc-LTBP3-9], AC008895.1 [ENSG00000279948.1], lnc-TBC1D3H-7 [lnc-TBC1D3H-7], lnc-TMEM250-3 [lnc-TMEM250-3], lnc-CDC42EP2-1 [lnc-CDC42EP2-1], AC087741.1 [ENSG00000262580.5], XLOC_156972 [XLOC_156972], lnc-PC-3 [lnc-PC-3], AC090589.3 [ENSG00000270060.1], XLOC_045084 [XLOC_045084], TIAF1 [ENSG00000221995.5], lnc-CYBA-4 [lnc-CYBA-4], lnc-SLC11A2-7 [lnc-SLC11A2-7], AC141586.1 [ENSG00000215154.6], AP003559.1 [ENSG00000256443.1], XLOC_095076 [XLOC_095076], PNPLA7 [ENSG00000130653.16], lnc-RNF166-5 [lnc-RNF166-5], XLOC_023911 [XLOC_023911], AC092127.1 [ENSG00000260417.1], lnc-TRPT1-3 [lnc-TRPT1-3], XLOC_028195 [XLOC_028195], XLOC_080106 [XLOC_080106], XLOC_026739 [XLOC_026739], lnc-NUP98-1 [lnc-NUP98-1], HDAC10 [ENSG00000100429.18], DRD4 [ENSG00000069696.7], lnc-DOC2B-3 [lnc-DOC2B-3], lnc-DOLK-1 [lnc-DOLK-1], CNIH2 [ENSG00000174871.11], RGL3 [ENSG00000205517.12], GALT [ENSG00000213930.11], AP001107.9 [ENSG00000255468.7], lnc-MKNK2-1 [lnc-MKNK2-1], AL033543.1 [ENSG00000279175.1].

In some embodiments, the Localization Domain promotes accumulation of the trans-splicing molecule to nuclear speckles. In some embodiments, the Localization Domain that promotes accumulation of the trans-splicing molecule to nuclear speckles is derived or isolated from a gene selected from the group consisting of: MALAT1 [NR_002819.4], MEG3[ENSG00000214548], XLOC_003526 [ENSG00000250657]. In some embodiments, the Localization Domain promotes accumulation of the trans-splicing molecule to nuclear speckles via binding to a protein selected from the group consisting of: SRSF1 [ENSG00000136450], SRSF2 [ENSG00000161547], SRSF3 [ENSG00000112081], SRSF4 [ENSG00000116350], SFSF6 [ENSG00000124193], SFSF7 [ENSG00000115875], SRSF10 [ENSG00000188529], SRSF11 [ENSG00000116754], CLK1 [ENSG00000013441], CLK2 [ENSG00000176444].

In some embodiments, the Localization Domain promotes accumulation of the trans-splicing molecule in nuclear speckles via association to a protein. In some embodiments, this protein is selected from group consisting of: ADNP [ENSG00000101126], ANXA7 [ENSG00000138279], API5 [ENSG00000166181], AQR [ENSG00000021776], ATAD2 [ENSG00000156802], BAZ1B [ENSG00000009954], BCLAF1 [ENSG00000029363], BTAF1 [ENSG00000095564], CCAR1 [ENSG00000060339], CCAR2 [ENSG00000158941], CDC5L [ENSG00000096401], CDC73 [ENSG00000134371], CDK11B [ENSG00000248333], CDK12 [ENSG00000167258], CDKN2AIP [ENSG00000168564], CHD3 [ENSG00000170004], CHD4 [ENSG00000111642], CHTF18 [ENSG00000127586], CPSF1 [ENSG00000071894], CSTF3 [ENSG00000176102], CTR9 [ENSG00000198730], CUL3 [ENSG00000036257], CUL4B [ENSG00000158290], CWC22 [ENSG00000163510], CWF19L1 [ENSG00000095485], DDX23 [ENSG00000174243], DDX39A [ENSG00000123136], DDX42 [ENSG00000198231], DDX46 [ENSG00000145833], DHX16 [ENSG00000204560], DHX38 [ENSG00000140829], DNMT1 [ENSG00000130816], ELOA [ENSG00000011007], EWSR1 [ENSG00000182944], FAF1 [ENSG00000185104], FBXO22 [ENSG00000167196], FKBP5 [ENSG00000096060], FUBP1 [ENSG00000162613], FUBP3 [ENSG00000107164], GPATCH8 [ENSG00000186566], GPS1 [ENSG00000169727], GTF3C1 [ENSG00000077235], GTF3C4 [ENSG00000125484], GTF3C5 [ENSG00000148308], HCFC1 [ENSG00000172534], HELLS [ENSG00000119969], IK [ENSG00000113141], ILF2 [ENSG00000143621], INTS13 [ENSG00000064102], KDM1A [ENSG00000004487], KHDRBS1 [ENSG00000121774], KHSRP [ENSG00000088247], LIGI [ENSG00000105486], MATR3 [ENSG00000280987], METTL1 [ENSG00000037897], MRE11 [ENSG00000020922], MSH2 [ENSG00000095002], MSH3 [ENSG00000113318], MSH6 [ENSG00000116062], NBN [ENSG00000104320], NCBP1 [ENSG00000136937], NONO [ENSG00000147140], PAF1 [ENSG00000006712], PDSSB [ENSG00000083642], POLD1 [ENSG00000062822], POLR2A [ENSG00000181222], POLR2B [ENSG00000047315], PPM1G [ENSG00000115241], PPP1R10 [ENSG00000204569], PRPF19 [ENSG00000110107], PRPF3 [ENSG00000117360], PRPF31 [ENSG00000105618], PRPF40A [ENSG00000196504], PRPF4B [ENSG00000112739], PRPF6 [ENSG00000101161], PSPC1 [ENSG00000121390], PTBP2 [ENSG00000117569], PUS7 [ENSG00000091127], RAD21 [ENSG00000164754], RAD50 [ENSG00000113522], RALY [ENSG00000125970], RBM10 [ENSG00000182872], RBM12 [ENSG00000244462], RBM14 [ENSG00000239306], RBM17 [ENSG00000134453], RBM25 [ENSG00000119707], RBM26 [ENSG00000139746], RBM4 [ENSG00000173933], RBMX [ENSG00000147274], RFC1 [ENSG00000035928], RFC4 [ENSG00000163918], RNF20 [ENSG00000155827], RNF40 [ENSG00000103549], RNMT [ENSG00000101654], RPL35A [ENSG00000182899], RPRD1B [ENSG00000101413], RPRD2 [ENSG00000163125], SAMHD1 [ENSG00000101347], SART1 [ENSG00000175467], SART3 [ENSG00000075856], SBNO1 [ENSG00000139697], SF3A1 [ENSG00000099995], SF3B1 [ENSG00000115524], SF3B2 [ENSG00000087365], SFPQ [ENSG00000116560], SIN3A [ENSG00000169375], SLC4A1AP [ENSG00000163798], SMARCC1 [ENSG00000173473], SMU1 [ENSG00000122692], SON [ENSG00000159140], STAG2 [ENSG00000101972], SUGT1 [ENSG00000165416], SUPTSH [ENSG00000196235], SUPT6H [ENSG00000109111], SYMPK [ENSG00000125755], TARDBP [ENSG00000120948], TCERG1 [ENSG00000113649], THOC2 [ENSG00000125676], THOC5 [ENSG00000100296], TP53BP1 [ENSG00000067369], TRMT1 [ENSG00000104907], TRMT1L [ENSG00000121486], TSR1 [ENSG00000167721], UBR5 [ENSG00000104517], UHRF1 [ENSG00000276043], USP39 [ENSG00000168883], USP48 [ENSG00000090686], USP7 [ENSG00000187555], WAC [ENSG00000095787], WDHD1 [ENSG00000198554], WRNIP1 [ENSG00000124535], XPO5 [ENSG00000124571], XPO7 [ENSG00000130227], XPOT [ENSG00000184575], YLPM1 [ENSG00000119596], ZC3H11A [ENSG00000058673], ZC3H14 [ENSG00000100722], ZMYND8 [ENSG00000101040], ZNF326 [ENSG00000162664].

In some embodiments, the RNA trans-splicing molecule comprises 1 Localization Domain. In some embodiments, the RNA trans-splicing molecule comprises 2 or more Localization Domains. In some embodiments, the trans-splicing RNA molecule comprises at least about 2, 3, 4, 5, 6, 7, 8, 9, 10, 11, 12, 13, 14, 15, 20, 30, 40, 50, 75, 100, 200, 300 or more Localization Domains.

### Untranslated Regions

In some embodiments, the nucleic acid encodes a 5′ untranslated region. The nucleic acid may comprise DNA. The nucleic acid comprising DNA may be transcribed into a trans-splicing molecule comprising RNA. The nucleic acid may comprise RNA. The nucleic acid comprising RNA may also be referred to as a trans-splicing nucleic acid. In some embodiments, the 5′ untranslated region increases the stability of the trans-splicing nucleic acid. In some embodiments, the 5′ untranslated region alters the localization of the trans-splicing nucleic acid. In some embodiments, the 5′ untranslated region alters the processing of the trans-splicing nucleic acid.

In some embodiments, the trans-splicing RNA further comprises a 3′ untranslated region. In some embodiments, the 3′ untranslated region increases the stability of the trans-splicing nucleic acid. In some embodiments, the 3′ untranslated region alters the localization of the trans-splicing nucleic acid. In some embodiments, the 3′ untranslated region alters the processing of the trans-splicing nucleic acid.

### Regulatory Elements

In some embodiments of the compositions of the disclosure, the nucleic acid may encode a promoter capable of expressing the trans-splicing RNA in a eukaryotic cell. In some embodiments of the compositions of the disclosure, the eukaryotic cell is an animal cell. In some embodiments, the animal cell is a mammalian cell. In some embodiments, the animal cell is a human cell.

### Enhancer Sequence

In some embodiments of the compositions of the disclosure, the nucleic acid may encode a trans-splicing enhancer sequence. The trans-splicing enhancer sequence may increase splicing efficiency so that the exonic sequence can be spliced to the target RNA with high efficiency. In other embodiments, the present disclosure provides a composition comprising a nucleic acid sequence encoding the trans-splicing RNA molecule.

In some embodiments, the trans-splicing enhancer sequences comprise 5′-X₁X₂X₃X₄X₅X₆-3′ wherein X₁ is uracil (U) or guanine (G); X₂ is adenine (A), uracil (U) or guanine (G); X₃ is adenine (A), uracil (U) and guanine (G); X₄ is adenine (A), uracil (U), cytosine (C) or guanine (G); X₅ is adenine (A), cytosine (C), uracil (U) or guanine (G); and X₆ is adenine (A), uracil (U) or guanine (G).

In some embodiments, the trans-splicing enhancer sequences comprise 5′-X₁X₂X₃X₄X₅X₆-3′ wherein; X₁ is selected from the group including adenine (A), uracil (U) and guanine (G); X₂ is selected from the group including adenine (A), uracil (U) and guanine (G); X₃ is selected from the group including adenine (A), uracil (U) and guanine (G); X₄ is selected from the group including adenine (A), uracil (U) and guanine (G); X₅ is selected from the group including adenine (A), uracil (U) and guanine (G); and X₆ is selected from the group including uracil (U) and guanine (G).

In some embodiments, the trans-splicing enhancer sequences comprise 5′-X₁X₂X₃X₄X₅X₆-3′ wherein; X₁ is selected from the group including adenine (A), uracil (U) and guanine (G); X₂ is selected from the group including uracil (U) and guanine (G); X₃ is selected from the group including adenine (A), uracil (U) and guanine (G); X₄ is selected from the group including uracil (U) and guanine (G); X₅ is selected from the group including uracil (U) and guanine (G); and X₆ is selected from the group including uracil (U) and guanine (G).

In some embodiments, the sequence encoding the trans-splicing enhancer is adjacent to the sequence encoding an exonic sequence. In some embodiments of the trans-splicing nucleic acid molecules as described herein, the trans-splicing enhancer sequence is directly adjacent to the exonic sequence. In some embodiments, the Intronic Domain comprises 1 trans-splicing enhancer sequence. In some embodiments, the Intronic Domain comprises 2 or more trans-splicing enhancer sequences. In some embodiments, the Intronic Domain comprises 3, 4, 5, 6, 7, 8, 9, 10, 11, 12, 13, 14, 15, 20, 30, 40, 50, 75, 100, 200, 300 or more trans-splicing enhancer sequences.

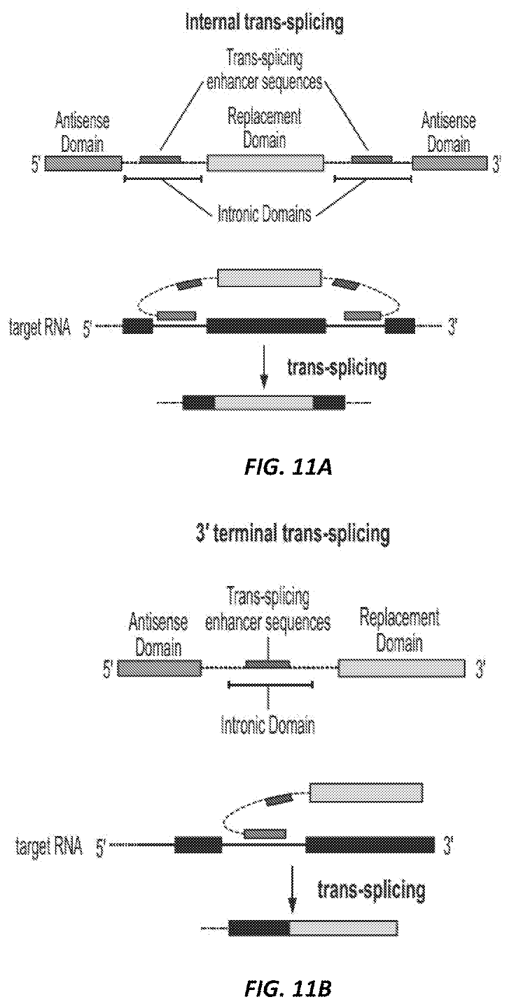

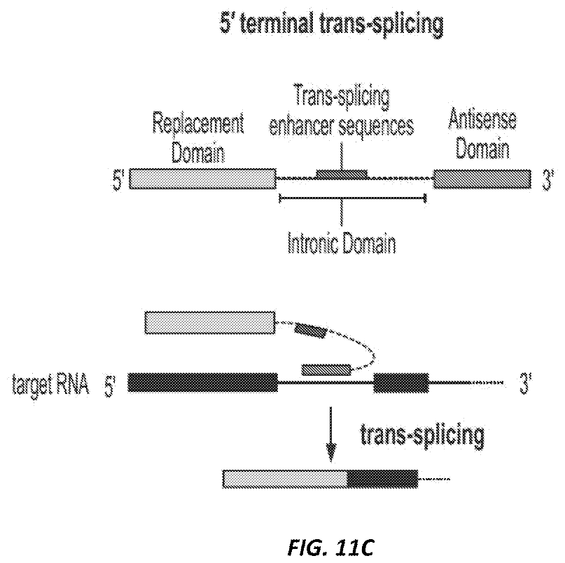

FIG. 11 illustrates three example embodiments of the compositions and methods described in this disclosure. FIG. 11A describes a double trans-splicing molecule which carries two antisense domains, one replacement domain, two intronic domains, and at least two trans-splicing enhancer sequences within the intronic domains. This design promotes replacement of an internal sequence within the target RNA while maintaining the adjacent 5′ and 3′ sequences around the replaced sequence. FIG. 11B illustrates the design of a 3′ terminal trans-splicing RNA that will replace the 3′ terminal end of a target RNA while maintaining the 5′ end. FIG. 11C illustrates the design of a 5′ terminal trans-splicing molecule that will replace the 5′ terminal end of a target RNA while maintaining the 3′ end.

### Stabilizing Sequence

The present disclosure provides a nucleic acid molecule encoding a stabilizing sequence. The nucleic acid molecule may comprise a ribonucleic acid (RNA), a deoxyribonucleic acid (DNA), or any combination thereof. The nucleic acid may encode an exonic sequence corresponding to a target RNA or porting thereof. A trans-splicing of the exonic sequence to the target RNA or portion thereof may correct the sequence. For example, the trans-splicing may correct a missing or mutated sequence of the target RNA. A barrier to efficient trans-splicing may be a cellular nuclease. To that end, blocking the activity of cellular nucleases may increase the effective level of the exonic sequence trans-spliced to the target RNA. The stabilizing sequence can alter the translation or stability of target RNAs to increase or decrease the production of a protein associated with the target RNA. The stabilizing sequence may be codon-optimized. Codon optimization refers to the fact that different cells differ in their usage of particular codons. This codon bias corresponds to a bias in the relative abundance of particular tRNAs in the cell type. By altering the codons in the sequence to match with the relative abundance of corresponding transfer RNAs (tRNAs), it is possible to increase expression. In some instances, it is also possible to decrease expression by deliberately choosing codons for which the corresponding tRNAs are rare in a particular cell type.

In some instances, stabilizing sequences are used to reduce degradation of the exonic sequence. A variety of RNA sequences derived from viruses, human and bacterial genes may block cellular exonuclease activity from the 3′ end (the exosome) or from the 5′ end (XRN1).

Compositions comprising stabilizing sequences disclosed herein include any sequences that promote trans-splicing. Examples of stabilizing sequences include sequences derived or isolated from the following flaviviral genomes without limitation: Apoi virus, Aroa virus, Bagaza virus, Banzi virus, Bouboui virus, Bukalasa bat virus, Cacipacore virus, Carey Island virus, Cowbone Ridge virus, Dakar bat virus, Dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Saint Louis encephalitis virus, Sal Vieja virus, San Perlita virus, Saumarez Reef virus, Sepik virus, Tembusu virus, Tick-borne encephalitis virus, Tyuleniy virus, Uganda S virus, Usutu virus, Wesselsbron virus, West Nile virus, Yaounde virus, Yellow fever virus, Yokose virus, Zika virus.

Examples of stabilizing sequences also include sequences derived or isolated from the following long non-coding RNA genes without limitation: CDKN2B-AS1 [NR_003529]; BANCR [NR_047671]; CASC15 [NR_015410]; CRNDE [NR_034105]; EMX2OS [NR_002791]; EVF2 [NR_015448]; FENDRR [NR_036444]; FTX [NR_028379]; GAS5 [NR_002578]; HOTAIR [NR_003716]; HOTAIRM1 [NR_038366]; HOXA-AS3 [NR_038832]; HOXA11-AS [NR_002795]; JPX [NR_024582]; LHX5-AS1 [NR_126425]; LINC01578 [NR_037600]; LINC00261 [NR_001558]; MALAT1 [NR_002819.4]; MEG3 [NR_046473]; TUNAR [NR_038861]; MIAT [NR_033320]; NEAT1 [NR_028272]; NR2F1-AS1 [NR_021490]; LINC-PINT [NR_015431]; PSMA3-AS1 [NR_029434]; EMX2OS [ENSG00000229847]; PVT1 [NR_003367]; MEG8 [NR_024149]; RMST [NR_024037]; SENCR [NR_038908]; SIX3-AS1 [NR_103786]; SOX21-AS1 [NR_046514]; TERC [NR_001566]; TUG1 [NR_002323]; XIST [NR_001564], malat1 [NR_002847.3], Nfx1 [NM_023739.3], Ogt [NM_139144.4], Nlrp6 [NM_133946.2], Mlxipl [NM_021455.5], Leng8 [NM_001374609.1], Gcgr [NM 008101.2], Gck [NM_001287386.1], Acly [NM 001199296.1], Ccnl1 [NM_001355433.1], Ccnl2 [NM 207678.2], Chkb [NM_007692.6], LINC1609, MEG3, LINCRNA-P21, LXRBSV, SRA, BACE1AS, IPW, MEG3, AIR, KCNQ1OT1, RMST, SNHG5, KCNQ1OT1, LINC1610, ADAPT33, SNHG3, GAS5, NEAT1, NEAT2, BACE1AS, KCNQ1OT1, MALAT1, RIAN, SNHG1, SNHG4, SNHG5, SNHG6, ZFAS1, MENβ, and Sno.

Examples of stabilizing sequences also include sequences derived or isolated from the genomes of the following viruses without limitation: Kaposi's sarcoma-associated herpesvirus, turnip yellow mosaic virus, Plautia stali intestine virus.

Examples of stabilizing sequences also include sequences that form pseudoknots, triplexes, or other tertiary RNA structures. In some embodiments, the stabilizing sequence forms a structure that blocks cellular nuclease activity. In some embodiments, the structure is a pseudoknot. In some embodiments, the structure is a stem-loop. In some embodiments, the stabilizing sequence blocks directional cellular exonuclease activity. In some embodiments, the stabilizing sequence forms a triplex that blocks 3′-5′ exonuclease activity. In some embodiments, the stabilizing sequence forms an exonuclease-resistant RNA that blocks 5′-3′ exonuclease activity.

### Enzyme Staple Molecule

Compositions of the present disclosure may comprise an enzyme staple molecule. In some embodiments of the compositions of the disclosure, the nucleic acid may encode a sequence configured to bind an enzyme staple molecule. In some embodiment, the enzyme staple molecule comprises an engineered small nuclear RNA (snRNA). In some embodiments, the snRNA is derived or isolated from a human spliceosomal snRNA gene. In some embodiments, the esnRNA domain is derived or isolated from a human small nuclear RNA gene is chosen from a group consisting of: U1, U2, U4, U5, U6, U7, U11, and U12. The sequence configured to bind the enzyme staple molecule may be derived from a human sequence. For example, the sequence configured to bind the enzyme staple molecule may be derived or isolated from a human snRNA gene. Examples of such snRNA may include U1, U2, U4, U5, U6, U7, U11, and U12. The engineered snRNA molecule may comprise sequences that are complementary to the exonic sequence. This complementary sequence may be located at or near the 5′ end of the engineered snRNA molecule. Complementary sequences incudes without limitation: 5′CGAGCTCTCT-3′ (SEQ ID NO: 4), 5′-AACGAGCTCT-3′ (SEQ ID NO: 5), 5′-CGCAACGAGC-3′ (SEQ ID NO: 6), 5′-TATCGCAACG-3′ (SEQ ID NO: 7), 5′-AATAATATCG-3′ (SEQ ID NO: 8), 5′-TAAGAGAGCT-3′ (SEQ ID NO: 9), 5′-AAGAGAGCTC-3′ (SEQ ID NO: 10), 5′-AGAGAGCTCGTTGC-3′ (SEQ ID NO: 11), 5′-GAGAGCTCGT-3′(SEQ ID NO: 12), 5′-AGAGCTCGTTGCGA-3′ (SEQ ID NO: 13), and 5′-GAGCTCGTTG-3′(SEQ ID NO: 14). In some embodiments, in the above complementary sequences, none, some, or all, of the thymidine bases may be replaced with uracil so that the trans-splicing enhancer sequences include without limitation: 5′-CGAGCUCUCU-3′ (SEQ ID NO: 15), 5′-AACGAGCUCU-3′ (SEQ ID NO: 16), 5′-CGCAACGAGC-3′ (SEQ ID NO: 17), 5′-UAUCGCAACG-3′ (SEQ ID NO: 18), 5′-AAUAAUAUCG-3′ (SEQ ID NO: 19), 5′-UAAGAGAGCU-3′(SEQ ID NO: 20), 5′-AAGAGAGCUC-3′ (SEQ ID NO: 21), 5′-AGAGAGCUCGUUGC-3′ (SEQ ID NO: 22), 5′-GAGAGCUCGU-3′ (SEQ ID NO: 23), 5′-AGAGCUCGUUGCGA-3′ (SEQ ID NO: 24), and 5′-GAGCUCGUUG-3′ (SEQ ID NO: 25).

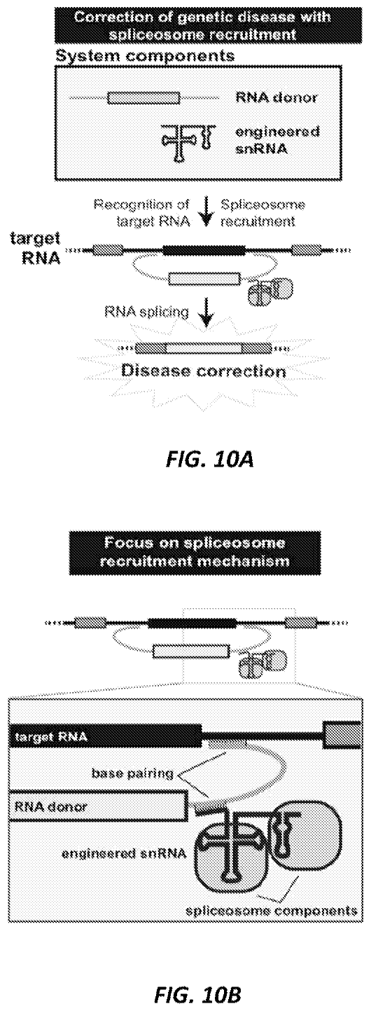

FIG. 10A illustrates a system composed of a donor RNA (e.g., a Replacement Domain encoding an exonic sequence that corresponds to a target RNA sequence or portion thereof) and an engineered small nuclear RNA (esnRNA). The combination of RNA donor molecule and esnRNA correct mutated RNAs via hybridization of the RNA donor to the target RNA carrying a mutation, followed by association of the esnRNA with the RNA donor, results in recruitment of spliceosome components and trans-splicing among the RNA donor molecule and the target RNA. This yields a corrected target RNA with the RNA donor molecule replacing a chosen sequence in the target RNA. FIG. 10B illustrates the how the components interact. Base pairing among the RNA donor and target RNA bring these molecule in close proximity. Base pairing among the esnRNA and the RNA donor brings spliceosome components in close proximity which promotes a trans-splicing reaction among the target RNA and the RNA donor.

In some embodiments, the sequence encoding an enzyme staple molecule comprises a sequence binding a stem-loop forming snRNA. In some embodiments, the stem-loop forming RNA sequences are derived or isolated from a human snRNA gene. In one embodiment, such a sequence has at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to its corresponding wild-type or originating sequence. In one embodiment, such a sequence has at most about 80%, at most about 85%, at most about 90%, at most about 91%, at most about 92%, at most about 93%, at most about 94%, at most about 95%, at most about 96%, at most about 97%, at most about 98%, at most about 99%, or 100% sequence identity to its corresponding wild-type or originating sequence.

In some embodiments the engineered snRNA or sequence thereof is not derived from any living organism, and is composed of a synthetic sequence that binds spliceosomal proteins.

### Tethering Protein

The present disclosure provides compositions comprising a protein. The protein may be a tethering protein. The tethering protein may promote association of the trans-splicing RNA and the target RNA and may comprise two domains: 1) a specific RNA binding domain that binds to the trans-splicing RNA, and 2) a second domain that promotes association of the trans-splicing RNA and the target RNA. This second domain can be isolated or derived from a non-specific double-stranded RNA binding domain that binds to and stabilizes the RNA-RNA duplex among the trans-splicing RNA and the target RNA. In this manner, the tethering protein increases the affinity of the trans-splicing RNA and target RNA duplex thereby increasing editing efficiency. Alternatively, the second domain can promote association with spliceosome components, thereby bringing the trans-splicing RNA molecule and the spliceosome-bound target RNA in close proximity. Alternatively, the second domain can promote association with a transcriptional complex, thereby bringing the trans-splicing RNA molecule and the recently-transcribed target RNA in close proximity. The tethering protein can comprise three domains: 1) a specific RNA binding domain that binds to the trans-splicing RNA, 2) a double-stranded RNA binding domain, and 3) a domain that promotes association with spliceosomal or transcriptional components.

Compositions comprising tethering proteins disclosed herein include any sequences that bind to the trans-splicing RNA and promote association of the trans-splicing RNA and a target RNA. Some non-limiting examples of tethering proteins comprise sequences that promote localization of trans-splicing molecules to the target RNA or to specific structures within the nucleus such as nuclear speckles or paraspeckles. Other non-limiting examples of tethering proteins comprise sequences that promote association of the trans-splicing molecule with nuclear-localized proteins and protein complexes such as the spliceosome, transcriptional proteins, or splicing factors.

The use of sequences derived from mRNA, long noncoding RNAs, and synthetic sequences may be used to alter that localization of varied transcript types within the cellular nucleus. Indeed, a variety of RNA sequences placed in a heterologous context promote the accumulation of RNAs in the nucleus or within specific structures in the nucleus such as nuclear speckles or paraspeckles.

In some embodiments, the tethering protein comprises two domains. In some embodiments, one domain is a specific RNA binding domain that is derived or isolated from a gene selected from the group consisting of: A1CF [ENSG00000148584], BOLL [ENSG00000152430], CELF1 [ENSG00000149187], CNOT4 [ENSG00000080802], CPEB1 [ENSG00000214575], DAZ3 [ENSG00000187191], DAZAP1 [ENSG00000071626], EIF4G2 [ENSG00000110321], ELAVL4 [ENSG00000162374], ESRP1 [ENSG00000104413], EWSR1 [ENSG00000182944], FUBP1 [ENSG00000162613], FUBP3 [ENSG00000107164], FUS [ENSG00000089280], HNRNPA0 [ENSG00000177733], HNRNPA2B1 [ENSG00000122566], HNRNPC [ENSG00000092199], HNRNPCL1 [ENSG00000179172], HNRNPD [ENSG00000138668], HNRNPDL [ENSG00000152795], HNRNPF [ENSG00000169813], HNRNPH2 [ENSG00000126945], HNRNPK [ENSG00000165119], HNRNPL [ENSG00000104824], IGF2BP1 [ENSG00000159217], IGF2BP2 [ENSG00000073792], ILF2 [ENSG00000143621], KHDRBS2 [ENSG00000112232], KHDRBS3 [ENSG00000131773], KHSRP [ENSG00000088247], MBNL1 [ENSG00000152601], MSI1 [ENSG00000135097], NOVA1 [ENSG00000139910], NUPL2 [None], PABPN1L [ENSG00000205022], PCBP1 [ENSG00000169564], PCBP2 [ENSG00000197111], PCBP4 [ENSG00000090097], PRR3 [ENSG00000204576], PTBP3 [ENSG00000119314], PUF60 [ENSG00000179950], PUM1 [ENSG00000134644], RALYL [ENSG00000184672], RBFOX2 [ENSG00000100320], RBFOX3 [ENSG00000167281], RBM15B [ENSG00000259956], RBM22 [ENSG00000086589], RBM23 [ENSG00000100461], RBM24 [ENSG00000112183], RBM25 [ENSG00000119707], RBM4 [ENSG00000173933], RBM41 [ENSG00000089682], RBM45 [ENSG00000155636], RBM47 [ENSG00000163694], RBM4B [ENSG00000173914], RBM6 [ENSG00000004534], RBMS2 [ENSG00000076067], RBMS3 [ENSG00000144642], RC3H1 [ENSG00000135870], SF1 [ENSG00000168066], SFPQ [ENSG00000116560], SNRPA [ENSG00000077312], SRSF10 [ENSG00000188529], SRSF11 [ENSG00000116754], SRSF2 [ENSG00000161547], SRSF4 [ENSG00000116350], SRSF5 [ENSG00000100650], SRSF8 [ENSG00000263465], SRSF9 [ENSG00000111786], TAF15 [ENSG00000270647], TARDBP [ENSG00000120948], TIA1 [ENSG00000116001], TRA2A [ENSG00000164548], TRNAU1AP [ENSG00000180098], UNK [ENSG00000132478], ZCRB1 [ENSG00000139168], ZFP36 [ENSG00000128016], ZNF326 [ENSG00000162664], SLBP [ENSG00000163950]. In some embodiments, the specific RNA binding domain is derived or isolated from a PUF, Pumby, or other human-derived engineered RNA binding protein.

In some embodiments, the tethering protein comprises a specific RNA binding domain and a non-specific double-stranded RNA binding domain. In some embodiments, the non-specific double-stranded RNA binding domain stabilizes the duplex among the trans-splicing RNA and the target RNA and comprises sequences isolated from a gene selected from the group consisting of: DGCR8 [ENSG00000128191], EIF2AK2 [ENSG00000055332], DICER1 [ENSG00000100697], ILF3 [ENSG00000129351], ADARB1 [ENSG00000197381], ADAR [ENSG00000160710], STAU2 [ENSG00000040341], STAU1 [ENSG00000124214], PRKRA [ENSG00000180228], EIF2AK2 [ENSG00000055332], RPS2 [ENSG00000140988], TRBP [ENSG00000139546], CDKN2AIP [ENSG00000168564], DHX9 [ENSG00000135829], NKRF [ENSG00000186416], MRPL44 [ENSG00000135900], DUS2 [ENSG00000167264], TARBP2 [ENSG00000139546], DROSHA [ENSG00000113360], IFIH1 [ENSG00000115267].

In some embodiments, the tethering protein comprises a specific RNA binding domain comprising sequences isolated or derived from SLBP and non-specific double-stranded RNA binding domain comprising sequences isolated or derived from TRBP. In some embodiments, the sequence from TRBP is amino acid residues 16-227. In some embodiments, the tethering fusion protein further comprises a glycine-serine linker and/or nuclear localization signals. In some embodiments, the tethering fusion protein comprises TRBP on the N-terminal side and SLBP on the C-terminal. In some embodiments, the tethering fusion protein comprises or consist of the following sequence (SEQ ID NO: 2): MPKKKRKVGGSLPSIEQMLAANPGKTPISLLQEYGTRIGKTPVYDLLKAEGQAHQPNFT FRVTVGDTSCTGQGPSKKAAKHKAAEVALKHLKGGSMLEPALEDSSSFSPLDSSLPEDIP VFTAAAAATPVPSVVLTRSPPMELQPPVSPQQSECNPVGALQELVVQKGWRLPEYTVTQ ESGPAHRKEFTMTCRVERFIEIGSGTSKKLAKRNAAAKMLLRVHTGGSGGSGGSGGSGG SGGSADFETDESVLMRRQKQINYGKNTIAYDRYIKEVPRHLRQPGIHPKTPNKFKKYSR RSWDQQIKLWKVALHFWDPKKKRKV. In some embodiments, the tethering fusion protein comprises at least about 60%, about 65%, about 70%, about 75%, about 80%, about 85%, about 90%, about 95%, about 97.5%, about 98%, about 99%, or about 100% identity with a sequence encoded by SEQ ID NO: 2. In some embodiments, the tethering fusion protein comprises SLBP on the N-terminal side and TRBP on the C-terminal. In some embodiments, the tethering fusion protein comprises or consist of the following sequence (SEQ ID NO: 3): MPKKKRKVADFETDESVLMRRQKQINYGKNTIAYDRYIKEVPRHLRQPGIHPKTPNKFK KYSRRSWDQQIKLWKVALHFWDGGSGGSGGSGGSGGSGGSGGSLPSIEQMLAANPGK TPISLLQEYGTRIGKTPVYDLLKAEGQAHQPNFTFRVTVGDTSCTGQGPSKKAAKHKAA EVALKHLKGGSMLEPALEDSSSFSPLDSSLPEDIPVFTAAAAATPVPSVVLTRSPPMELQP PVSPQQSECNPVGALQELVVQKGWRLPEYTVTQESGPAHRKEFTMTCRVERFIEIGSGTS KKLAKRNAAAKMLLRVHTPKKKRKV. In some embodiments, the tethering fusion protein comprises at least about 60%, about 65%, about 70%, about 75%, about 80%, about 85%, about 90%, about 95%, about 97.5%, about 98%, about 99%, or about 100% identity with a sequence encoded by SEQ ID NO: 3.

In some embodiments, the tethering protein comprises a specific RNA binding domain and a domain that associates with the spliceosome. In some embodiments, the tethering protein comprises a specific RNA binding domain, a domain that associates with the spliceosome, and a domain the binds non-specifically to double-stranded RNA. In some embodiments, the tethering protein comprises a domain derived from a component of the spliceosome. In some embodiments, the domain that associates with the spliceosome comprises sequences isolated from a gene selected from the group consisting of: CASC3 [ENSG00000108349], EIF4A3 [ENSG00000141543], MAGOH [ENSG00000162385], MAGOHB [ENSG00000111196], RBM8A [ENSG00000265241], LSM 1.00 [ENSG00000175324], LSM 2.00 [ENSG00000204392], LSM 3.00 [ENSG00000170860], LSM 4.00 [ENSG00000130520], LSM 5.00 [ENSG00000106355], LSM 6.00 [ENSG00000164167], LSM 7.00 [ENSG00000130332], LSM 8.00 [ENSG00000128534], LSM 10.00 [ENSG00000181817], LSM 11.00 [ENSG00000155858], LSM 12.00 [ENSG00000161654], LSM14A [ENSG00000257103], LSM14B [ENSG00000149657], NAA38 [ENSG00000183011], BCAS2 [ENSG00000116752], CDC5L [ENSG00000096401], CTNNBL1 [ENSG00000132792], CWC15 [ENSG00000150316], PLRG1 [ENSG00000171566], PRPF19 [ENSG00000110107], SF3A1 [ENSG00000099995], SF3A2 [ENSG00000104897], SF3A3 [ENSG00000183431], PHF5A [ENSG00000100410], SF3B1 [ENSG00000115524], SF3B2 [ENSG00000087365], SF3B3 [ENSG00000189091], SF3B4 [ENSG00000143368], SF3B5 [ENSG00000169976], SF3B6 [ENSG00000115128], SNRPB [ENSG00000125835], SNRPD1 [ENSG00000167088], SNRPD2 [ENSG00000125743], SNRPD3 [ENSG00000100028], SNRPD3 [ENSG00000286070], SNRPE [ENSG00000182004], SNRPF [ENSG00000139343], SNRPG [ENSG00000143977], SNRPN [ENSG00000128739], CCAR1 [ENSG00000060339], CHERP [ENSG00000085872], DDX46 [ENSG00000145833], DHX15 [ENSG00000109606], HNRNPAB [ENSG00000197451], HNRNPA1 [ENSG00000135486], PRPF40A [ENSG00000196504], PUF60 [ENSG00000179950], RBM5 [ENSG00000003756], RBM10 [ENSG00000182872], RBM17 [ENSG00000134453], RBM25 [ENSG00000119707], SF1 [ENSG00000168066], SMNDC1 [ENSG00000119953], SUGP1 [ENSG00000105705], THRAP3 [ENSG00000054118], U2AF1 [ENSG00000160201], U2AF2 [ENSG00000063244], U2SURP [ENSG00000163714], AQR [ENSG00000021776], BUD31 [ENSG00000106245], CRNKL1 [ENSG00000101343], DHX15 [ENSG00000109606], IK [ENSG00000113141], ISY1 [ENSG00000240682], MFAP1 [ENSG00000140259], PPIE [ENSG00000084072], PPIL1 [ENSG00000137168], PQBP1 [ENSG00000102103], PRPF38A [ENSG00000134748], RBM22 [ENSG00000086589], SMU1 [ENSG00000122692], SNW1 [ENSG00000100603], TFIP11 [ENSG00000100109], WBP4 [ENSG00000120688], WBP11 [ENSG00000084463], XAB2 [ENSG00000076924], ZMAT2 [ENSG00000146007], AQR [ENSG00000021776], BUD31 [ENSG00000106245], CCDC12 [ENSG00000160799], CDC40 [ENSG00000168438], CRNKL1 [ENSG00000101343], CWC22 [ENSG00000163510], CWC25 [ENSG00000273559], CWC27 [ENSG00000153015], DHX16 [ENSG00000204560], EFTUD2 [ENSG00000108883], EIF4A3 [ENSG00000141543], GPATCH1 [ENSG00000076650], GPKOW [ENSG00000068394], ISY1 [ENSG00000240682], PPIE [ENSG00000084072], PPIL1 [ENSG00000137168], PPIL2 [ENSG00000100023], PRCC [ENSG00000143294], PRPF8 [ENSG00000174231], RBM22 [ENSG00000086589], RNF113A [ENSG00000125352], RNU5A-1 [ENSG00000199568], RNU6-1 [ENSG00000206625], SAP18 [ENSG00000150459], SNRNP40 [ENSG00000060688], SNRNP200 [ENSG00000144028], SNW1 [ENSG00000100603], XAB2 [ENSG00000076924], ZNF830 [ENSG00000198783], AQR [ENSG00000021776], BCAS2 [ENSG00000116752], BUD31 [ENSG00000106245], CACTIN [ENSG00000105298], CCDC12 [ENSG00000160799], CDC5L [ENSG00000096401], CDC40 [ENSG00000168438], CDK10 [ENSG00000185324], CRNKL1 [ENSG00000101343], CTNNBL1 [ENSG00000132792], CWC15 [ENSG00000150316], CWC22 [ENSG00000163510], CWC27 [ENSG00000153015], STEEP1 [ENSG00000018610], DDX41 [ENSG00000183258], DHX8 [ENSG00000067596], DHX16 [ENSG00000204560], DHX35 [ENSG00000101452], EFTUD2 [ENSG00000108883], FAM32A [ENSG00000105058], FAM50A [ENSG00000071859], FRA10AC1 [ENSG00000148690], GPATCH1 [ENSG00000076650], GPKOW [ENSG00000068394], HNRNPC [ENSG00000092199], HSPA8 [ENSG00000109971], ISY1 [ENSG00000240682], LENG1 [ENSG00000105617], NOSIP [ENSG00000142546], PLRG1 [ENSG00000171566], PPIE [ENSG00000084072], PPIG [ENSG00000138398], PPIL1 [ENSG00000137168], PPIL3 [ENSG00000240344], PPWD1 [ENSG00000113593], PRPF8 [ENSG00000174231], PRPF18 [ENSG00000165630], PRPF19 [ENSG00000110107], RBM22 [ENSG00000086589], RNF113A [ENSG00000125352], RNU5A-1 [ENSG00000199568], RNU6-1 [ENSG00000206625], SAP18 [ENSG00000150459], SDE2 [ENSG00000143751], SLU7 [ENSG00000164609], SNRNP40 [ENSG00000060688], SNRNP200 [ENSG00000144028], SNW1 [ENSG00000100603], SRRM2 [ENSG00000167978], SYF2 [ENSG00000117614], WDR83 [ENSG00000123154], XAB2 [ENSG00000076924], ZNF830 [ENSG00000198783], DDX39B [ENSG00000198563], SF1 [ENSG00000168066], U2AF1 [ENSG00000160201], U2AF2 [ENSG00000063244], AQR [ENSG00000021776], BUD13 [ENSG00000137656], BUD31 [ENSG00000106245], CACTIN [ENSG00000105298], CCDC12 [ENSG00000160799], CDC40 [ENSG00000168438], CDK10 [ENSG00000185324], CRNKL1 [ENSG00000101343], CWC22 [ENSG00000163510], CWC27 [ENSG00000153015], STEEP1 [ENSG00000018610], C90RF78 [ENSG00000136819], DDX41 [ENSG00000183258], DHX8 [ENSG00000067596], DHX16 [ENSG00000204560], EFTUD2 [ENSG00000108883], EIF4A3 [ENSG00000141543], ESS2 [ENSG00000100056], FAM32A [ENSG00000105058], FAM50A [ENSG00000071859], GPKOW [ENSG00000068394], HNRNPC [ENSG00000092199], HSPA8 [ENSG00000109971], ISY1 [ENSG00000240682], LENG1 [ENSG00000105617], MAGOH [ENSG00000162385], NOSIP [ENSG00000142546], PPIE [ENSG00000084072], PPIG [ENSG00000138398], PPIL1 [ENSG00000137168], PPIL3 [ENSG00000240344], PPWD1 [ENSG00000113593], PRPF8 [ENSG00000174231], RBM8A [ENSG00000265241], RBM22 [ENSG00000086589], RNF113A [ENSG00000125352], RNU5A-1 [ENSG00000199568], RNU6-1 [ENSG00000206625], SDE2 [ENSG00000143751], SLU7 [ENSG00000164609], SNIP1 [ENSG00000163877], SNRNP40 [ENSG00000060688], SNRNP200 [ENSG00000144028], SNW1 [ENSG00000100603], SRRM2 [ENSG00000167978], SYF2 [ENSG00000117614], WDR83 [ENSG00000123154], XAB2 [ENSG00000076924], ZNF830 [ENSG00000198783], RBM42 [ENSG00000126254], SART1 [ENSG00000175467], SNRNP27 [ENSG00000124380], USP39 [ENSG00000168883], RNU1-1 [ENSG00000206652], SNRNP70 [ENSG00000104852], SNRPA [ENSG00000077312], SNRPC [ENSG00000124562], RNU2-1 [ENSG00000274585], SF3A1 [ENSG00000099995], SF3A2 [ENSG00000104897], SF3A3 [ENSG00000183431], SNRPA1 [ENSG00000131876], SNRPB2 [ENSG00000125870], PPIH [ENSG00000171960], PRPF3 [ENSG00000117360], PRPF4 [ENSG00000136875], PRPF31 [ENSG00000105618], RNU4-1 [ENSG00000200795], RNU6-1 [ENSG00000206625], SNU13 [ENSG00000100138], CD2BP2 [ENSG00000169217], DDX23 [ENSG00000174243], EFTUD2 [ENSG00000108883], PRPF6 [ENSG00000101161], PRPF8 [ENSG00000174231], RNU5A-1 [ENSG00000199568], SNRNP40 [ENSG00000060688], SNRNP200 [ENSG00000144028], TXNL4A [ENSG00000141759].

In some embodiments, the tethering protein comprises a specific RNA binding domain and a domain that associates with a protein involved in transcription. In some embodiments, the tethering protein comprises a specific RNA binding domain, a domain that associates with a protein involved in transcription, and a domain the binds non-specifically to double-stranded RNA. In some embodiments of the compositions of the disclosure, the tethering protein comprises a domain that binds to RNA polymerase II. In some embodiments, the domain that associates with a protein involved in transcription comprises sequences isolated from a gene selected from the group consisting of: CCNH [ENSG00000134480], CDK7 [ENSG00000134058], MNAT1 [ENSG00000020426], GTF2A1 [ENSG00000165417], GTF2A1L [ENSG00000242441], GTF2A2 [ENSG00000140307], TAF1 [ENSG00000147133], TAF2 [ENSG00000064313], TAF3 [ENSG00000165632], TAF4 [ENSG00000130699], TAF5 [ENSG00000148835], TAF6 [ENSG00000106290], TAF7 [ENSG00000178913], TAF8 [ENSG00000137413], TAF9 [ENSG00000273841], TAF10 [ENSG00000166337], TAFR1 [ENSG00000064995], TAF12 [ENSG00000120656], TAF13 [ENSG00000197780], TBP [ENSG00000112592], GTF2E1 [ENSG00000153767], GTF2E2 [ENSG00000197265], GTF2F1 [ENSG00000125651], GTF2F2 [ENSG00000188342], ERCC2 [ENSG00000104884], ERCC3 [ENSG00000163161], GTF2H1 [ENSG00000110768], GTF2H2 [ENSG00000145736], GTF2H2 [ENSG00000183474], GTF2H3 [ENSG00000111358], GTF2H4 [ENSG00000213780], GTF2H5 [ENSG00000272047], BDP1 [ENSG00000145734], BRF1 [ENSG00000185024], TBP [ENSG00000112592], GTF3C1 [ENSG00000077235], GTF3C2 [ENSG00000115207], GTF3C3 [ENSG00000119041], GTF3C4 [ENSG00000125484], GTF3C5 [ENSG00000148308], GTF3C6 [ENSG00000155115], GTF2B [ENSG00000137947], TBP [ENSG00000112592], GTF2I [ENSG00000263001], TCEA1 [ENSG00000187735], GTF3A [ENSG00000122034], BRF1 [ENSG00000185024], CCNC [ENSG00000112237], CDK8 [ENSG00000132964], CDK19 [ENSG00000155111], MED1 [ENSG00000125686], MED29 [ENSG00000063322], MED27 [ENSG00000160563], MED4 [ENSG00000136146], MED24 [ENSG00000008838], MED6 [ENSG00000133997], MED7 [ENSG00000155868], MED8 [ENSG00000159479], MED9 [ENSG00000141026], MED10 [ENSG00000133398], MED11 [ENSG00000161920], MED12 [ENSG00000184634], MED12L [ENSG00000144893], MED13 [ENSG00000108510], MED13L [ENSG00000123066], MED14 [ENSG00000180182], MED15 [ENSG00000099917], MED16 [ENSG00000175221], MED17 [ENSG00000042429], MED18 [ENSG00000130772], MED19 [ENSG00000156603], MED20 [ENSG00000124641], MED21 [ENSG00000152944], MED22 [ENSG00000148297], MED23 [ENSG00000112282], MED25 [ENSG00000104973], MED26 [ENSG00000105085], MED28 [ENSG00000118579], MED30 [ENSG00000164758], MED31 [ENSG00000108590].

### Efficiency

The present disclosure provides compositions that increase the efficiency of an RNA trans-splicing. The efficiency of RNA trans-splicing is defined as the fraction of a target RNA molecule that experiences a specific change in sequence composition that is mediated by trans-splicing. This efficiency measurement is an important metric of therapeutic efficacy. Factors affecting efficiency of trans-splicing may include the association between a trans-splicing RNA molecules and a target RNA molecule. Aspects of the present disclosure provide a nucleic acid encoding an exonic sequence. The nucleic acid may comprise DNA. The nucleic acid comprising DNA may be transcribed into RNA, e.g., a trans-splicing RNA molecule comprising the exonic sequence. The exonic sequence may correspond to a sequence or portion thereof of a target RNA. The exonic sequence may associate with the target RNA. The exonic sequence may be trans-spliced into the sequence of the target RNA. The sequence of the target RNA may be mutated or missing a sequence. The trans-splicing of the exonic sequence to the target RNA may correct the missing or mutated sequence of the target RNA. The nucleic acid may comprise RNA, e.g., the nucleic acid may be a trans-splicing molecule. Spliceosome-mediated RNA trans-splicing may require association of the trans-splicing RNA and a target RNA molecule within a cellular nucleus with sufficient proximity and affinity to support assembly and activity of a spliceosome. The association of the trans-splicing molecule and the target RNA molecule may enhance the trans-splicing of the exonic sequence to the sequence of the target RNA molecule.

Compositions and methods for RNA trans-splicing as disclosed herein may involve inclusion of an RNA-binding protein. The RNA-binding protein may be a tethering protein for trans-splicing molecules. Tethering proteins as disclosed herein may confer RNA-trans-splicing with high efficiency against multiple RNA targets. RNA trans-splicing systems as disclosed herein may have numerous advantages over other trans-splicing systems. For example, RNA trans-splicing systems as disclosed herein may confer higher efficiency than other RNA trans-splicing systems. The improved efficiency can replace defective RNA sequences at levels sufficient to reconstitute the activity of mutated genes to treat recessive genetic disorders. The improved efficiency of RNA trans-splicing systems as disclosed herein can replace defective RNA sequences at levels higher than those in other trans-splicing systems. For example, treatment of many recessive gene disorders may require at least 30% efficiency where 100% is complete replacement of a sequence within a Target RNA. Trans-splicing systems as disclosed herein may be able to achieve at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or higher levels of efficiency. Trans-splicing systems as disclosed herein may also be able to replace defective RNA sequences at levels sufficient to treat dominant genetic disorders. Trans-splicing systems as disclosed herein may also be able to replace defective RNA sequences at levels sufficient at levels higher than those in other trans-splicing systems. As a single mutated allele is sufficient to cause disease, many diseases in this class may require highly-efficient replacement of mutated sequences as the mutated sequences can cause toxicity. As a result, even higher efficiency is required (70%+). Finally, RNA trans-splicing systems as disclosed herein may have the ability to modify multiple Target RNAs. RNA trans-splicing systems as disclosed herein may efficiently replace sequences with multiple target RNAs.

Trans-splicing systems as disclosed herein may increase the production of one or more proteins by one or more target mRNAs. Trans-splicing systems as disclosed herein may increase the production of one or more proteins by one or more target mRNAs with a high level of efficiency. Trans-splicing systems as disclosed herein may be able to achieve at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or higher levels of efficiency. By contrast, small molecule drugs that increase translation by promoting stop codon read-through may suffer extensive off-targets due to promotion of read through on non-target mRNAs. Further, pre-mature stop codons are only one of many causes of insufficient protein levels. Engineered tRNAs to block pre-mature termination codons suffer from this same fundamental issue. An RNA trans-splicing system, in contrast, can replace sequences in any target mRNA with translation-amplifying sequences to increase protein production.

The combination of a tethering protein with a trans-splicing RNA is a general capability that further allows the alteration of non-coding sequences within target RNAs. By replacing the 5′ or 3′ untranslated regions of target RNAs with high efficiency, this allows the alteration of RNA behaviors such as translation or turnover. The net result of these effects is increased production of protein from Target RNAs or other downstream effects associated with altered RNA levels.

The present disclosure provides, in some embodiments, a composition comprising a trans-splicing RNA molecule and a tethering protein that promotes association of the trans-splicing RNA molecule and a target RNA. In some embodiments, the tethering protein is a tethering fusion protein. In some embodiments, described herein is a trans-splicing RNA that promotes a trans-splicing reaction with a target RNA molecule and a tethering protein that promotes association of the trans-splicing RNA and the target RNA. In some embodiments, described herein is are vectors, compositions and cells comprising or encoding the trans-splicing RNA molecule and tethering protein. In some embodiments, described herein is are methods of using the trans-splicing RNA molecule, vectors, compositions and cells of the disclosure to treat a disease or disorder.

### Systems

The present disclosure provides systems comprising any of the compositions or nucleic acids as disclosed herein. The nucleic acid may encode an exonic domain corresponding to a sequence or portion thereof of a target RNA molecule. The sequence of the target RNA molecule may be mutated or missing a sequence. A trans-splicing of the exonic sequence to the target RNA may correct the mutated or missing sequence of the target RNA. The nucleic acid may encode an intronic domain. The nucleic acid may encode an antisense domain. The nucleic acid may encode a localization domain, e.g., a nuclear localization domain. The nucleic acid may encode or comprise one or more untranslated regions, e.g., a 3′ untranslated region or a 5′ untranslated region. The nucleic acid may encode a regulatory element. Systems as described herein may further comprise a protein that promotes an association of the exonic sequence to the sequence of the target RNA. The protein may be a tethering protein. The protein may be a fusion protein. In some embodiments, the systems described herein further comprise an enzyme. In some embodiments, the enzyme comprises a spliceosome enzyme. In some embodiments, the enzyme comprises a transcriptional enzyme. In some embodiments, the system comprises an enzyme configured to insert the replacement domain into the Target mRNA molecule. In some embodiments, the systems described comprise a binding protein configured to interact with a transcriptional enzyme couple to the target mRNA.

In some embodiments, the system does not comprise a CRISPR/Cas enzyme.

In some embodiments, the system comprises an engineered small nuclear RNA derived or isolated from a snRNA. In some embodiments, the snRNA is U1, U2, U4, U5, U6, U7, U11, or U12. In some embodiments, the snRNA is U1. In some embodiments, the small nucleic RNA promotes trans-splicing between a target RNA and a trans-splicing RNA.

### Nucleic Acids

Also provided herein are nucleic acid sequences encoding the trans-splicing nucleic acids disclosed herein for use in gene transfer and expression techniques described herein. Nucleic acids as disclosed herein may comprise any of the domains, sequences, or elements as disclosed herein. For example, nucleic acids as disclosed herein may encode an intronic domain. Nucleic acids as disclosed herein may encode an antisense domain. Nucleic acids as disclosed herein may encode a replacement domain or an exonic sequence. Nucleic acids as disclosed herein may encode a localization domain. Nucleic acids as disclosed herein may encode an untranslated region. Nucleic acids as disclosed herein may encode a regulatory element. Nucleic acids as disclosed herein may encode a sequence configured to bind an enzyme staple molecule. Nucleic acids as disclosed herein may encode a trans-splicing enhancer sequence. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical nucleic acid sequence to the reference nucleic acid sequence when compared using sequence identity methods run under default conditions. Specific sequences are provided as examples of particular embodiments. Additionally, an equivalent polynucleotide is one that hybridizes under stringent conditions to the reference polynucleotide or its complement.

The nucleic acid sequences (e.g., polynucleotide sequences) disclosed herein may be codon-optimized. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are rare in a particular cell type, such as through codon usage tables. Based on the genetic code, nucleic acid sequences coding for various replacement domains can be generated. In some embodiments, such a sequence is optimized for expression in a host or target cell, such as a host cell used to express the trans-splicing RNA comprising a replacement domain in which the disclosed methods are practiced (such as in a mammalian cell, e.g., a human cell). Codon preferences and codon usage tables for a particular species can be used to engineer isolated nucleic acid molecules encoding a replacement domain (such as one encoding a protein having at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type protein) that takes advantage of the codon usage preferences of that particular species. For example, the replacement domains disclosed herein can be designed to have codons that are preferentially used by a particular organism of interest. In one example, a replacement domain nucleic acid sequence is optimized for expression in human cells, such as one having at least 70%, at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type or originating nucleic acid sequence. In some embodiments, an isolated trans-splicing nucleic acid molecule encoding at least one replacement domain (which can be part of a vector) includes at least one replacement domain coding sequence that is codon optimized for expression in a eukaryotic cell, or at least one replacement domain coding sequence codon optimized for expression in a human cell. In one embodiment, such a codon optimized replacement domain coding sequence has at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a replacement domain having at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type or originating protein. In another embodiment, a variety of clones comprising functionally equivalent nucleic acids may be routinely generated, such as nucleic acids which differ in sequence but which encode the same replacement domain protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H.5 Freeman and Co., NY, which is incorporated herein by reference in its entirety).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogsteen binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC;

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, less than 35% identity, less than 30% identity, less than 25% identity, or less than 20% identity with one of the sequences of the present invention.

### Cells and Tissues

The present disclosure provides compositions, nucleic acids, and systems for trans-splicing, which may be administered to a cell or to a tissue. In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell. In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, an immune cell of the disclosure is a lymphocyte. In some embodiments, an immune cell of the disclosure is a T lymphocyte (also referred to herein as a T-cell). Examples of T-cells of the disclosure include, but are not limited to, naïve T cells, effector T cells, helper T cells, memory T cells, regulatory T cells (Tregs) and Gamma delta T cells. In some embodiments, an immune cell of the disclosure is a B lymphocyte. In some embodiments, an immune cell of the disclosure is a natural killer cell. In some embodiments, an immune cell of the disclosure is an antigen-presenting cell.

In some embodiments of the compositions and methods of the disclosure, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell.

In some embodiments of the compositions and methods of the disclosure, a somatic cell of the disclosure is an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the compositions and methods of the disclosure, a brain cell of the disclosure is a neuronal cell. In some embodiments, a neuron cell of the disclosure is a neuron of the central nervous system. In some embodiments, a neuron cell of the disclosure is a neuron of the brain or the spinal cord. In some embodiments, a neuron cell of the disclosure is a neuron of a cranial nerve or an optic nerve. In some embodiments, a neuron cell of the disclosure is a neuron of the peripheral nervous system. In some embodiments, a neuron cell of the disclosure is a neuroglial or a glial cell. In some embodiments, a glial of the disclosure is a glial cell of the central nervous system including, but not limited to, oligodendrocytes, astrocytes, ependymal cells, and microglia. In some embodiments, a glial of the disclosure is a glial cell of the peripheral nervous system including, but not limited to, Schwann cells and satellite cells.

In some embodiments of the compositions and methods of the disclosure, a liver cell of the disclosure is a hepatocytes. In some embodiments, a liver cell of the disclosure is a hepatic stellate cell. In some embodiments, a liver cell of the disclosure is Kupffer cell. In some embodiments, a liver cell of the disclosure is a sinusoidal endothelial cells.

In some embodiments of the compositions and methods of the disclosure, a retinal cell of the disclosure is a photoreceptor. In some embodiments, a photoreceptor cell of the disclosure is a rod. In some embodiments, a retinal cell of the disclosure is cone. In some embodiments, a retinal cell of the disclosure is a bipolar cell. In some embodiments, a retinal cell of the disclosure is a ganglion cell. In some embodiments, a retinal cell of the disclosure is a horizontal cell. In some embodiments, a retinal cell of the disclosure is an amacrine cell.

In some embodiments of the compositions and methods of the disclosure, a heart cell of the disclosure is a cardiomyocyte. In some embodiments, a heart cell of the disclosure is a cardiac pacemaker cell.

In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Methods

The present disclosure provides methods for enhancing trans-splicing. Trans-splicing is a significant step in the process of protein production. Incorrect messenger RNA(mRNA) sequences may lead to incorrect protein production, e.g., incorrect amino acid sequence or misfolding. To that end, any of the composition, systems, and nucleic acids as disclosed herein may be used in any of the methods as disclosed herein to enhance trans-splicing of an exonic sequence to a target RNA sequence or portion thereof to correct the target RNA sequence or portion thereof. For example, the target RNA sequence or portion thereof may comprise a missing or mutated sequence. Methods as disclosed herein may comprise providing a nucleic acid encoding the exonic sequence. Methods as disclosed herein may comprise providing any of the tethering proteins as disclosed herein. The tethering protein may enhance an association of the exonic sequence to the target RNA sequence or portion thereof. Methods as disclosed herein may be used to, e.g., correct an amino acid sequence, correct protein or polypeptide misfolding, increase protein production, or decrease protein production.

In certain aspects, described herein are methods of associating an exonic sequence with a target RNA. The present disclosure provides a nucleic acid encoding the exonic sequence. The nucleic acid may comprise DNA. The nucleic acid comprising DNA may be transcribed into RNA, e.g., a trans-splicing RNA molecule comprising the exonic sequence. The nucleic acid may comprise RNA. The nucleic acid comprising RNA may be a trans-splicing RNA molecule. In some embodiments, the methods comprise providing a trans-splicing RNA molecule as described herein and binding a tethering protein as described herein to the trans-splicing RNA molecule. In some embodiments, the method comprise providing said trans-splicing RNA molecule comprising: a replacement domain; and an intronic domain comprising a site configured to interact with an RNA binding protein, wherein said intronic domain is configured to promote insertion of said replacement domain into a target mRNA molecule; and binding a tethering protein to said one or more RNA-binding protein sites that promote RNA splicing and to said target RNA molecule to associate said trans-splicing RNA molecule with said target RNA molecule. In some embodiments, the methods comprise providing said trans-splicing RNA molecule, wherein said trans-splicing RNA molecule comprises: a replacement domain; and an intronic domain comprising a site configured to promote insertion of said replacement domain into a target mRNA molecule; and binding a tethering protein to said trans-splicing RNA molecule and to said target RNA molecule to associate said trans-splicing RNA molecule with said target RNA molecule, wherein said trans-splicing RNA molecule lacks a CRISPR-associated protein.

In certain aspects, described herein is a method for promoting trans-splicing. In some embodiments, the methods comprise providing a trans-splicing RNA molecule as described herein and using an enzyme as described herein to insert the replacement domain into a target RNA molecule. In some embody, the methods comprise providing a trans-splicing RNA molecule as described herein and interacting a binding protein as described herein with a transcriptional enzyme coupled to said target mRNA.

In certain embodiments, the methods comprise providing a trans-splicing ribonucleic acid (RNA) molecule comprising: a replacement domain; and an intronic domain; and using an enzyme to insert said replacement domain into a target mRNA molecule. In some embodiments, the method comprises: providing a trans-splicing ribonucleic acid (RNA) molecule comprising: a replacement domain; and an intronic domain; and using an enzyme to insert said replacement domain into a target mRNA molecule, wherein said trans-splicing RNA molecule lacks a CRISPR-associated enzyme. In some embodiments, the method comprises: providing a trans-splicing ribonucleic acid (RNA) molecule comprising: a replacement domain; and an intronic domain; and interacting an RNA-binding protein with a transcriptional enzyme coupled to said target mRNA. In some embodiments, the trans-splicing RNA molecule comprises one or more binding sites configured to interact with an RNA-binding protein. In some embodiments, the RNA-binding protein is a tethering protein. In some embodiments, the RNA-binding protein is encoded by one or more human-derived sequences. In some embodiments, the RNA-binding protein comprises one or more domains configured to interact with the transcriptional enzyme. In some embodiments, the RNA-binding protein comprises one or more domains configured to interact with the enzyme configured to insert the replacement domain into the target mRNA molecule. In some embodiments, the methods comprise “providing a trans-splicing ribonucleic acid (RNA) molecule comprising: a replacement domain; and an intronic domain; and interacting a binding protein with a transcriptional enzyme coupled to said target mRNA, wherein said system for trans-splicing lacks a CRISPR-associated protein. In some embodiments, the tethering protein is a tethering protein.

The tethering protein promotes association of the trans-splicing RNA and target RNA by one of the following mechanisms: 1) stabilizing the RNA-RNA duplex among the trans-splicing RNA and the target RNA, 2) promoting transport of the trans-splicing RNA to the location of the target RNA at the site of spliceosome assembly, or 3) promoting transport of the trans-splicing RNA to the location of the target RNA at the site of transcription. As the tethering protein is derived from human protein exclusively, this avoids the risk of adaptive immune response associated with non-human protein such as engineered RNA binding proteins or CRISPR proteins. Further, the tethering protein is designed in a manner that is compatible with any target RNA and does not require redesign for individual target RNAs. This is achieved by use of a non-specific double-stranded RNA binding protein within the tethering protein that stabilizes RNA-RNA duplexes of any nucleobase composition. As a result, a single tethering protein can be used for any target RNA thereby increasing the applicability and utility of the approach.

This combination of trans-splicing RNA with a tethering protein promotes RNA trans-splicing in a manner that is sufficient to replace disease-causing RNA sequences in human cells to address disease. Indeed, low efficiency has been a major barrier to many nucleic acid editing approaches including RNA trans-splicing. The disclosure provides compositions and methods for specifically targeting disease-causing RNA molecules and replacing disease-causing RNA sequences within these RNA molecules with high efficiency. The trans-splicing RNA molecule implementations show utility in a variety of contexts including replacement of disease-causing sequences or insertion of engineered sequences into Target RNAs. The engineered sequences can alter the translation or stability of Target RNAs to increase or decrease protein production or Target RNA levels. This disclosure provides vectors, compositions and cells comprising or encoding the trans-splicing RNA and methods of using the trans-splicing RNA compositions.

The tethering protein can non-specifically promote association of the trans-splicing and target RNA. Rather than rely upon a protein that promotes association of the trans-splicing RNA with a specific target RNA, the tethering protein can promote association with an arbitrary target RNA so that a single tethering protein design can be used in the context of multiple target RNAs. As programmability is a central feature of RNA-targeting technology, this a useful activity in the context of trans-splicing. Further, by comprising human-derived protein sequences, the tethering protein avoids immunogenicity issues that may arise through the use of non-human or bacteria-derived proteins.

In one aspect, described herein is an RNA technology that enables replacement of arbitrary sequences within specific RNA molecules in living cells. The technology, based on RNA trans-splicing, utilizes the naturally-existing spliceosome in human cells to provide the catalytic activity for this trans-splicing process. RNA splicing occurs within RNA molecules where exons are concatenated and introns removed from immature messenger RNA molecules (pre-mRNAs) to form mature messenger RNA molecules (mRNAs). This process is referred to as cis-splicing and requires the set of enzymes and noncoding RNAs collectively known as the spliceosome. RNA trans-splicing is a process by which the spliceosome concatenates exons derived from distinct and separate RNA molecules. Described herein are compositions that increase the efficiency of RNA trans-splicing. These improved RNA trans-splicing compositions can be used to replace mutated sequences within a target RNA molecule to address a human disease. Replacement of arbitrary RNA sequences is a general ability with innumerable specific applications a few of which have been explored as relevant demonstrations. RNA trans-splicing can insert engineered sequences into a target RNA to impart new activities to the target RNA such as altered RNA stability or altered RNA translation. This feature can be used to increase production of protein by a target RNA. In the broadest sense, this RNA trans-splicing technology can impart arbitrary changes to both coding and non-coding regions of target RNAs.

In one aspect, described herein is a trans-splicing RNA molecule and a tethering protein comprising two domains: a specific RNA binding domain and a non-specific RNA binding domain. The non-specific RNA binding domain associates with an RNA-RNA duplex in a sequence non-specific fashion. The specific RNA binding domain associates with a sequence present in the trans-splicing RNA.

In one aspect, described herein is a trans-splicing RNA molecule and a tethering protein comprising two domains: a specific RNA binding domain and a spliceosome-binding protein. The spliceosome binding protein associates with the spliceosome which is in close proximity to the target RNA, thereby increasing trans-splicing among the target RNA and the trans-splicing RNA.

In one aspect, described herein is a trans-splicing RNA molecule and a tethering protein comprising two domains: a specific RNA binding domain and a transcriptional-component protein. The spliceosome binding protein associates with the a transcriptional enzyme which is in close proximity to the target RNA, thereby increasing trans-splicing among the target RNA and the trans-splicing RNA.

In some embodiments, the methods comprise administering to a subject in need thereof, a therapeutically effective amount of a treatment comprising the systems described herein. In some embodiments, the subject is afflicted with, diagnosed, or suspected to have, a genetic disease. In some embodiments, the disease comprises myotonic dystrophy, Duchenne muscular dystrophy, Dravet syndrome.

### Delivery of Compositions, Systems, and Nucleic Acids

The present disclosure provides modes of delivering any of the compositions, systems, and nucleic acids described herein. The mode may comprise use of a vector, liposome, lipoplex, nanoparticle, or any combination thereof.

### Vectors

The present disclosure provides vectors that may comprise or encode any of the nucleic acids disclosed herein. In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11 or AAV12. In some embodiments, the viral vector is replication incompetent. In some embodiments, the viral vector is isolated or recombinant (rAAV). In some embodiments, the viral vector is self-complementary (scAAV). In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 0.3 kb to 4.75 kb. In some embodiments, AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh74 vector, a modified AAV.rh74 vector, an AAV.rh64R1 vector, and a modified AAV.rh64R1 vector and any combinations or equivalents thereof.

In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. In some embodiments, lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mngabey simian immunodeficiency virus (SIVSM) vector, a modified sooty mangabey simian immunodeficiency virus (SIVSM) vector, a African green monkey simian immunodeficiency virus (SIVAGM) vector, a modified African green monkey simian immunodeficiency virus (SIVAGM) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex, an exosome or a dendrimer. In some embodiments, the vector is an expression vector or recombinant expression system. As used herein, the term “recombinant expression system” refers to a genetic construct for the expression of certain genetic material formed by recombination.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Expression control elements include but are not limited to promoters, enhancers, microRNAs, post-transcriptional regulatory elements, polyadenylation signal sequences, 5′ or 3′ untranslated regions, and introns.

Expression control elements may be constitutive, inducible, repressible, or tissue-specific, for example. A “promoter” is a control sequence that is a region of a polynucleotide sequence at which initiation and rate of transcription are controlled. It may comprise genetic elements at which regulatory proteins and molecules may bind such as RNA polymerase and other transcription factors. In some embodiments, expression control by a promoter is tissue-specific. Non-limiting examples of promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nP2, PPE, ENK, EAAT2, GFAP, MBP, H1 and U6 promoters. In some embodiments, the promoter is a sequence isolated or derived from a promoter capable of driving expression of a transfer RNA (tRNA). In some embodiments, the promoter is isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter. In some embodiments, the promoter is isolated or derived from a valine tRNA promoter.

An “enhancer” is a region of DNA that can be bound by activating proteins to increase the likelihood or frequency of transcription. Non-limiting examples of enhancers and post-transcriptional regulatory elements include the CMV enhancer and WPRE.

In some embodiments of the compositions and methods of the disclosure, an expression vector, viral vector or non-viral vector provided herein, includes without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

In some embodiments of the compositions and methods of the disclosure, a vector comprises or encodes a trans-splicing nucleic acid of the disclosure. In some embodiments, the vector comprises or encodes at least one trans-splicing nucleic acid of the disclosure. In some embodiments, the vector comprises or encodes one or more trans-splicing nucleic acid(s) of the disclosure. In some embodiments, the vector comprises or encodes two or more trans-splicing nucleic acids of the disclosure.

### Liposomes, Lipoplexes and Nanoparticles

The present disclosure provides liposomes, lipoplexes and nanoparticles for delivering any of the compositions, nucleic acids, or system as described herein.

In some embodiments, the liposome, lipoplex, or nanoparticle can further comprise a non-cationic lipid, a PEG conjugated lipid, a sterol, or any combination thereof.

In some embodiments, the the liposome, lipoplex, or nanoparticle further comprises a non-cationic lipid, wherein the non-ionic lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine and non-cationic lipids described, for example, in WO2017/099823 or US2018/0028664.

In some embodiments, the liposome, lipoplex, or nanoparticle further comprises a conjugated lipid, wherein the conjugated lipid, wherein the conjugated-lipid is selected from the group consisting of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-l-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt.

In some embodiments, the liposome, lipoplex, or nanoparticle further comprises cholesterol or a cholesterol derivative.

In some embodiments, the liposome, lipoplex, or nanoparticle further comprises an ionizable lipid, a non-cationic lipid, a conjugated lipid that inhibits aggregation of particles, and a sterol. The amount of the ionizable lipid, the non-cationic lipid, the conjugated lipid that inhibits aggregation of particles, and the sterol can be varied independently. In some embodiments, the lipid nanoparticle comprises an ionizable lipid in an amount from about 20 mol % to about 90 mol % of the total lipid present in the particle, a non-cationic lipid in an amount from about 5 mol % to about 30 mol % of the total lipid present in the particle, a conjugated lipid that inhibits aggregation of particles in an amount from about 0.5 mol % to about 20 mol % of the total lipid present in the particle, and a sterol in an amount from about 20 mol % to about 50 mol % of the total lipid present in the particle.

The ratio of total lipid to DNA vector can be varied as desired. For example, the total lipid to DNA vector (mass or weight) ratio can be from about 10:1 to about 30:1.

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the term “coupled” may refer to a weak or strong interaction between two or more atoms or molecules. The interaction may be directly or indirectly mediated by one or more molecules.

### EXAMPLES

The following examples are included for illustrative purposes only and are not intended to limit the scope of the invention.

### Example 1: Identifying Tethering Fusion Proteins

### Background

The present disclosure describes specific RNA sequences or non-specific double-stranded RNA sequences for use in any of the compositions, systems, and methods as disclosed herein. The present disclosure describes the use of protein domains in the context of RNA trans-splicing. The present disclosure describes the role of these protein domains in binding RNA in a heterologous context. For example, the protein domains may exist on a tethering fusion protein. This study analyzes the role of such tethering fusion proteins comprising RNA binding domains.

### Materials and Methods

First, combinations of tethering fusion proteins comprising a specific RNA binding domain and a non-specific double-stranded RNA binding domain are systematically assessed to test if they could increase the association among a trans-splicing RNA and a target RNA and therefore increase trans-splicing efficiency. Varied tethering fusion proteins are compared in combination with a trans-splicing molecule carrying binding sites for the specific RNA binding domain that targets a split GFP reporter RNA that fluoresces after successful activity of the RNA trans-spicing molecule. A GFP reporter is used to compare the relative influence of different sequences on the efficiency of the trans-splicing reaction. The tethering fusion proteins that are compared comprise at least one of the following non-specific double-stranded RNA binding protein domains (with selected residues in parentheses): TRBP(16-227), STAU1 (182-360), STAU1 (1-360), MDA5 (306-1025), RIG1 (230-925), ADAR (518-818). The tethering fusion proteins that are compared comprise at least one of the following specific double-stranded RNA binding protein domains: a PUF protein, or SLBP.

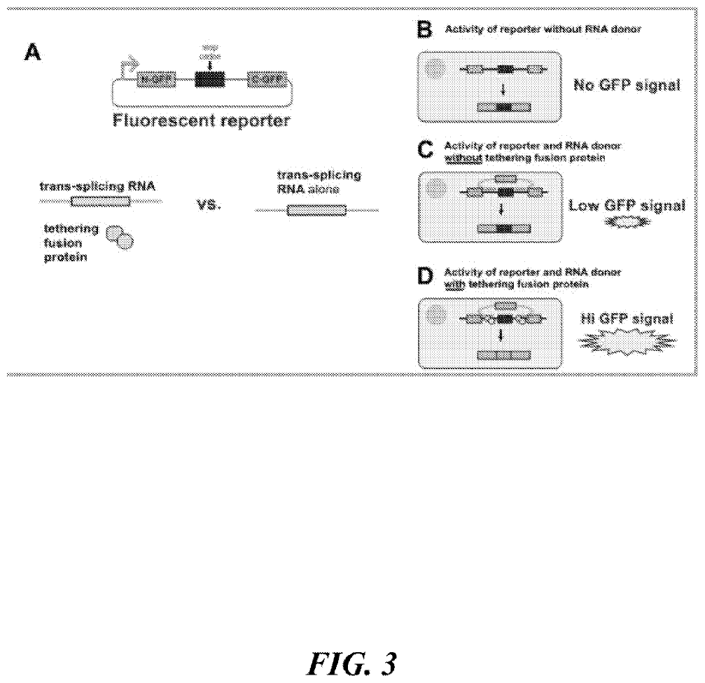

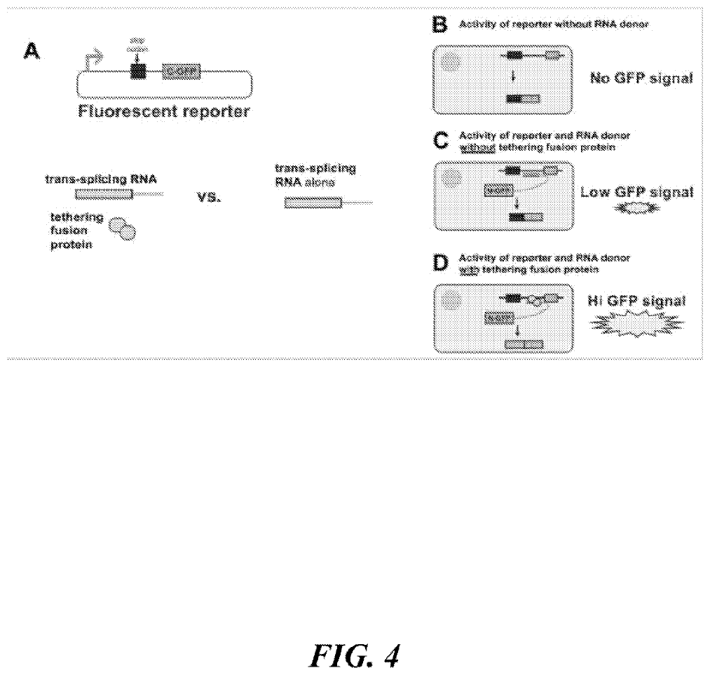

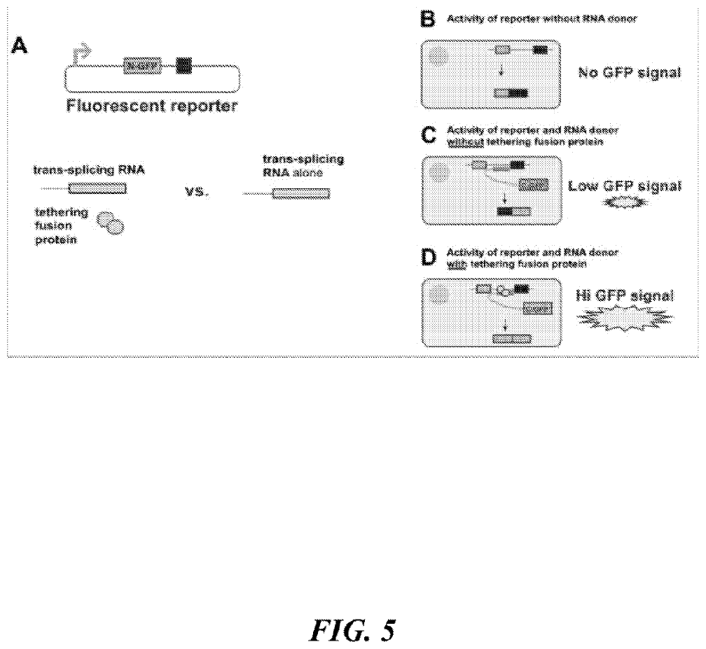

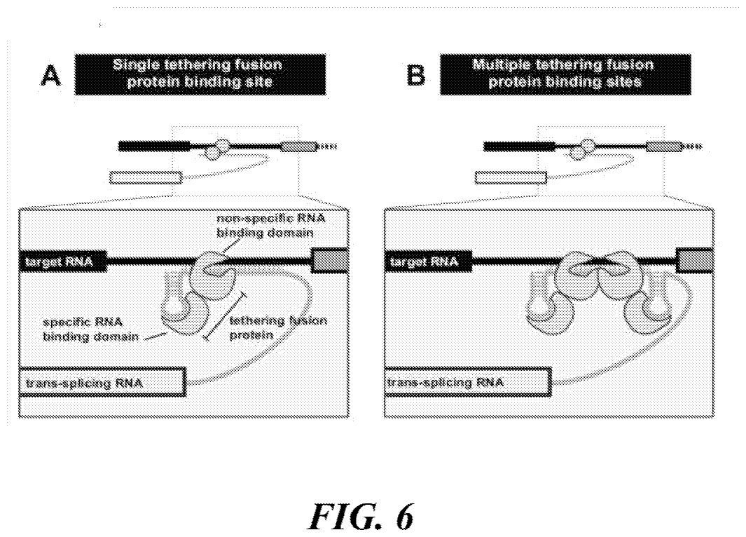

FIGS. 3-5 depict a schematic of the plasmids used in the trans-splicing activity assays.

### Results

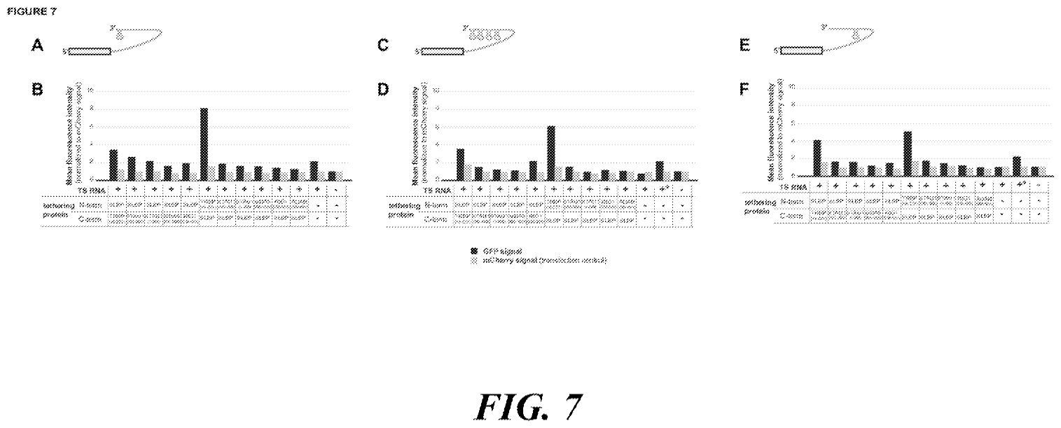

Experiments are conducted with in HEK293 cells transiently-transfected plasmids encoding the trans-splicing RNA, the tethering protein, and the reporter with data reported in FIGS. 7-9. Measurements were conducted with fluorescence-activated cell sorting

FIG. 7 illustrates that SLBP fused to the double-stranded RNA binding domains of TRBP increases trans-splicing activity the most compared to other tethering fusion proteins and to a control trans-splicing RNA that lacks SLBP binding sites. The figure legend describes whether a trans-splicing RNA is present (“+” indicates that a trans-splicing molecule with SLBP sites is present, “+*” indicates a trans-splicing molecule lacking SLBP sites is present, and “−” indicates that no trans-splicing molecule is present). The figure legend also describes the identities of the N- and C-terminal portions of the tethering fusion protein where parenthetical amino acid numbering indicates that a specific portion of the referenced protein is present.

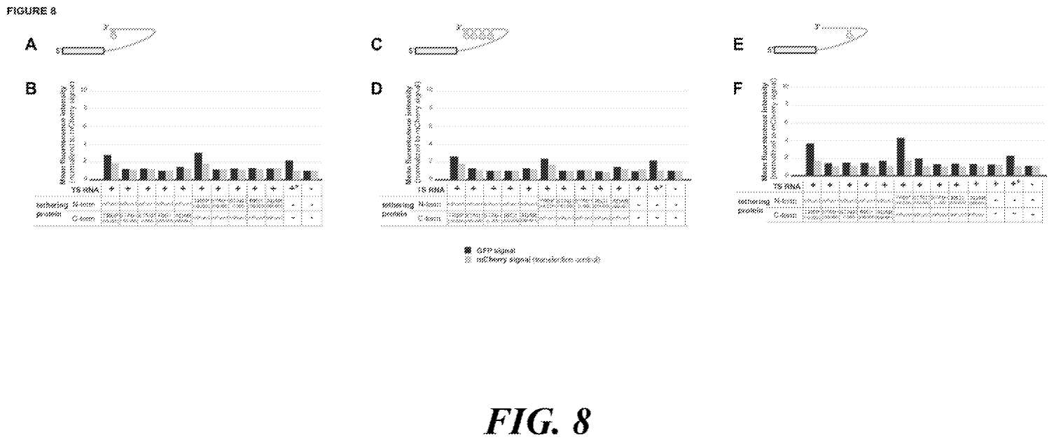

FIG. 8 illustrates that mPum1 fused to the double-stranded RNA binding domains of TRBP increases trans-splicing activity the most compared to other tethering fusion proteins and to a control trans-splicing RNA that lacks mPum1 binding sites.

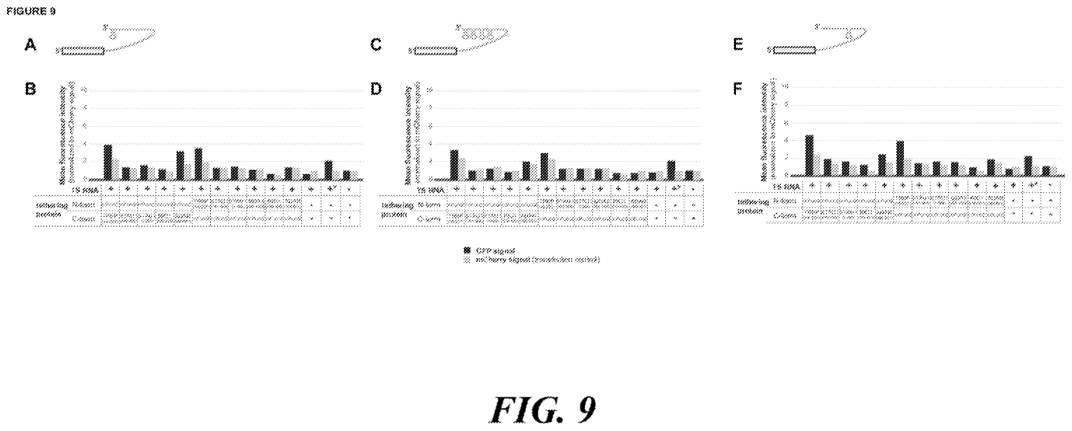

FIG. 9 illustrates that mPum2 fused to the double-stranded RNA binding domains of TRBP increases trans-splicing activity the most compared to other tethering fusion proteins and to a control trans-splicing RNA that lacks mPum2 binding sites.

### Example 2: Assessing the In-Vivo Effect of Tethering Fusion Proteins on Trans-Spicing Molecule Efficiency in Cell Lines

In order to further investigate the activity of tethering fusion protein sequences on trans-splicing efficiency, experiments are conducted to measure the efficiency editing of two endogenous genes: Scn1a and Dmd. Mutations in these genes cause Dravet syndrome and Duchenne muscular dystrophy, respectively. Cell lines (Neuro-2A, C2C12) that express these genes are transfected with trans-splicing molecules which target each of these genes along with tethering fusion proteins in order to assess trans-splicing efficiency. RNA is extracted from these cells 48 hours later and subjected to RNA reverse transcription and quantitative PCR using primers that amplify the trans-splicing molecule and a housekeeping gene.

### Example 3: Assessing the In-Vivo Effect of Tethering Fusion Proteins on Trans-Spicing Molecule Efficiency in a Model of Dravet Syndrome

In order to further investigate the activity of the tethering fusion protein on trans-splicing molecule efficiency, experiments are conducted in a mouse models of Dravet syndrome. Specifically, mice carrying mutations in exon 1 of Scn1a that display frequent and fatal seizures (129S-Scn1atm1Kea/Mmjax) EW treated with adeno-associated virus (AAV) encoding trans-splicing molecules and tethering fusion proteins. AAV IA administered via direct brain injection or via intracerebroventricular injection within the first month of life. Next, seizure frequency and survival of mice is measured. Mice treated with AAV encoding the trans-splicing RNA and tethering fusion proteins display reduced seizure frequency and greater survival than untreated mice or mice treated with a control AAV that did not comprise a tethering fusion protein.

### Example 4: Assessing the In-Vivo Effect of Tethering Fusion Proteins on Trans-Spicing Molecule Efficiency in a Model of Duchenne Muscular Dystrophy Syndrome

In order to further investigate the activity of the trans-splicing RNA and tethering fusion protein, experiments are conducted in a mouse models of Duchenne muscular dystrophy syndrome. Mice carrying mutations in exon 10 of Dmd that experience muscle degeneration and eventual death (B6Ros.Cg-Dmdmdx-5Cv/J) are treated with adeno-associated virus (AAV) encoding trans-splicing RNAs and tethering fusion proteins. AAV is administered via intramuscular injection or via systemic injection within the first month of life. Next, various measurements of muscle strength such as rotorod assay and survival of mice are measured. Mice treated with AAV encoding the trans-splicing RNA and tethering fusion protein display increased strength and greater survival than untreated mice or mice treated with a control AAV that did not comprise a tethering fusion protein

### Example 5: Use of Tethering Fusion Proteins and Trans-Splicing to Increase the Translation of Specific Target RNAs

Myotonic dystrophy is caused by RNAs that carry repetitive ‘CUG’ tracts that bind the splicing factor MBNL1. Titration of MBNL1 away from its targets causes widespread dysfunction of RNA alternative splicing and is responsible for most manifestations of disease in patients. Increasing MBNL1 protein production with an efficient RNA trans-splicing approach could address this disease via production of sufficient MBNL1 protein to reconstitute its activities in alternative splicing regulation.

To assess the ability of an RNA trans-splicing systems comprising tethering fusion proteins to increase protein production from specific mRNAs, an RNA trans-splicing system carrying tethering fusion proteins and a Woodchuck Hepatitis Virus (WHV) post-transcriptional Regulatory Element (WPRE) is created, as well as a reporter that comprises a firefly luciferase coding sequence (pMIR-GLO luciferase) and the last 2 exons and intervening intron of MBNL1.

Experiments are conducted with either transiently-transfected reporter and trans-splicing molecule or systems packaged in lentivirus. Some tethering fusion proteins do not increase trans-splicing. Other tethering fusion proteins increase trans-splicing.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A system for trans-splicing, comprising:
a. a nucleic acid molecule encoding:
i. an exonic sequence; and
ii. at least one intronic domain configured to promote insertion of said exonic sequence into a target RNA molecule; and
iii. one or more binding domains configured to interact with an RNA-binding protein, wherein said RNA-binding protein is encoded by one or more human-derived sequences; and
b. said RNA binding protein, which is configured to insert said exonic sequence into said target RNA molecule.

2. The system for trans-splicing of claim 1, wherein said intronic domain comprises said one or more binding domains.

3. The system for trans-splicing of claim 1 or 2, wherein the RNA binding protein is a tethering protein.

4. The system for trans-splicing of claim 3, wherein said tethering protein comprises: (a) an RNA-binding domain that binds to a specific sequence encoded by said nucleic acid molecule; and (b) a non-specific double-stranded RNA binding domain that stabilizes the RNA-RNA hybridization between said specific sequence and said target RNA.

5. The system for trans-splicing of claim 4, wherein said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1.

6. The system for trans-splicing of claim 4, wherein said RNA-binding domain that binds to said specific sequence encoded by said nucleic acid molecule comprises sequences isolated or derived from a PUF or Pumby protein.

7. The system for trans-splicing of claim 4, wherein said RNA-binding domain that binds to said specific sequence encoded by said nucleic acid molecule comprises sequences isolated or derived from the gene SLBP.

8. The system for trans-splicing of claim 3, wherein said tethering protein further comprises a domain configured to associate with an enzyme configured to insert said exonic sequence into said target mRNA molecule.

9. The system for trans-splicing of claim 8, wherein said tethering protein further comprises an RNA-binding domain configured to bind a specific sequence encoded by said nucleic acid molecule.

10. The system for trans-splicing of claim 3, wherein said tethering protein is isolated or derived from human protein sequences.

11. The system for trans-splicing of any one of the preceding claims, further comprising an engineered small nuclear RNA derived or isolated from a U1 snRNA gene that promotes trans-splicing between a target RNA and said exonic sequence.

12. The system for trans-splicing of any one of the preceding claims, wherein said nucleic acid molecule encodes one or more binding sites for the RNA-binding domain.

13. The system for trans-splicing of any one of the preceding claims, wherein said nucleic acid molecule further encodes a sequence that promotes accumulation of the exonic sequence molecule in the cellular nucleus.

14. The system for trans-splicing of claim 13, wherein said sequence that promotes accumulation of the exonic sequence molecule in the cellular nucleus is derived or isolated from a long noncoding RNA.

15. The system for trans-splicing of any one of the preceding claims, wherein said nucleic acid molecule further encodes a 3′ untranslated region that increases the stability of the trans-splicing molecule.

16. The system for trans-splicing of any one of the preceding claims, wherein said nucleic acid molecule further encodes a 5′ untranslated region that increases the stability of the trans-splicing molecule.

17. The system for trans-splicing of any one of the preceding claims, wherein said nucleic acid molecule further encodes a gene expression-enhancing element.

18. The system for trans-splicing of claim 17, wherein said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element.

19. The system for trans-splicing of any one of the preceding claims, wherein said nucleic acid molecule further encodes a heterologous promoter.

20. The system for trans-splicing of any one of the preceding claims, wherein said system for trans-splicing lacks a CRISPR-associated protein.

21. A vector comprising the system for trans-splicing of any one of the preceding claims.

22. The vector of claim 21, wherein said vector is selected from the group consisting of: adeno-associated virus, retrovirus, lentivirus, adenovirus, nanoparticle, micelle, liposome, lipoplex, polymersome, polyplex, and dendrimer.

23. A cell comprising the vector of claim 21.

24. A method for treating a disease comprising administering to a patient in need of a therapeutically effective amount of a treatment comprising a nucleic acid molecule according to any one of the preceding claims.

25. A system for trans-splicing, comprising:
a. a nucleic acid molecule encoding:
i. an exonic sequence; and
ii. at least one intronic domain configured to promote insertion of said exonic sequence into a target RNA molecule; and
b. a protein configured to insert said exonic sequence into said target RNA molecule,
wherein said system for trans-splicing lacks a CRISPR-associated protein.

26. The system for trans-splicing of claim 25, wherein said nucleic acid molecule encodes one or more binding sites for said protein.

27. The system for trans-splicing of claim 26, wherein said protein is a tethering protein.

28. The system for trans-splicing of claim 27, wherein said tethering protein is a fusion tethering protein.

29. The system for trans-splicing of claim 27, wherein said tethering protein comprises: (a) an RNA-binding domain that binds to a specific sequence encoded by said nucleic acid molecule; and (b) a domain configured to associate with a transcriptional or spliceosomal protein.

30. The system for trans-splicing of claim 29, wherein said tethering protein further comprises a domain that binds non-specifically to double-stranded RNA that stabilizes the RNA-RNA hybridization between said specific sequence encoded by said nucleic acid molecule and said target RNA.

31. The system for trans-splicing of claim 27, wherein said tethering protein comprises: (a) an RNA-binding domain that binds to a specific sequence encoded by said nucleic acid molecule; and (b) a non-specific double-stranded RNA binding domain that stabilizes the RNA-RNA hybridization between said specific sequence and said target RNA.

32. The system for trans-splicing of claim 31, wherein said non-specific double-stranded RNA binding domain comprises sequences isolated or derived from a gene selected from the group consisting of DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1.

33. The system for trans-splicing of claim 31, wherein said RNA-binding domain that binds to said specific sequence encoded by said nucleic acid molecule comprises sequences isolated or derived from a PUF or Pumby protein.

34. The system for trans-splicing of claim 31, wherein said RNA-binding domain that binds to said specific sequence encoded by said nucleic acid molecule comprises sequences isolated or derived from the gene SLBP.

35. The system for trans-splicing of claim 27, wherein said tethering protein is isolated or derived from human protein sequences.

36. The system for trans-splicing of any one of claims 25-35, Further comprising an engineered small nuclear RNA derived or isolated from a Ut snRNA gene that promotes trans-splicing between a target RNA and said exonic sequence.

37. The system for trans-splicing of any one of claims 25-36, wherein said nucleic acid molecule encodes one or more binding sites for the RNA-binding domain.

38. The system for trans-splicing of any one of claims 25-37, wherein said nucleic acid molecule further encodes a sequence that promotes accumulation of the exonic sequence molecule in the cellular nucleus.

39. The system for trans-splicing of claim 38, wherein said sequence that promotes accumulation of the exonic sequence molecule in the cellular nucleus is derived or isolated from a long noncoding RNA.

40. The system for trans-splicing of any one of claims 25-39, wherein said nucleic acid molecule further encodes a 3′ untranslated region that increases the stability of the trans-splicing molecule.

41. The system for trans-splicing of any one of claims 25-40, wherein said nucleic acid molecule further encodes a 5′ untranslated region that increases the stability of the trans-splicing molecule.

42. The system for trans-splicing of any one of claims 25-41, wherein said nucleic acid molecule further encodes a gene expression-enhancing element.

43. The system for trans-splicing of claim 42, wherein said gene expression-enhancing element comprises a sequence derived or isolated from the group consisting of Woodchuck Hepatitis Virus (WHV) Posttranscriptional Regulatory Element (WPRE), triplex from MALAT1, the PRE of Hepatitis B virus (HPRE), and an iron response element.

44. The system for trans-splicing of any one of claims 25-43, wherein said nucleic acid molecule further encodes a heterologous promoter.

45. A vector comprising the system for trans-splicing of any one of claims 25-44.

46. The vector of claim 45, wherein said vector is selected from the group consisting of: adeno-associated virus, retrovirus, lentivirus, adenovirus, nanoparticle, micelle, liposome, lipoplex, polymersome, polyplex, and dendrimer.

47. A cell comprising the vector of claim 45.

48. A method for treating a disease comprising administering to a patient in need of a therapeutically effective amount of a treatment comprising a nucleic acid molecule according to any one of claims 25-47.

49. A method for correcting a genetic defect in a subject comprising administering to said subject a nucleic acid molecule according to any one of claims 25-48.

50. A nucleic acid molecule encoding:
a. an exonic sequence;
b. at least one intronic domain configured to promote insertion of said exonic sequence into a target RNA molecule; and
c. one or more binding domains that interact with an RNA-binding protein, wherein said RNA-binding protein is encoded by one or more human-derived sequences, and wherein said RNA-binding protein is configured to interact with a transcriptional or spliceosomal enzyme coupled to said target RNA.

51. The nucleic acid molecule of claim 50, wherein said system for trans-splicing lacks a CRISPR-associated protein.

52. The nucleic acid molecule of claim 50, wherein said RNA-binding protein is a tethering protein.

53. The nucleic acid molecule of claim 51, wherein said tethering protein is a tethering fusion protein.

54. The nucleic acid molecule of claim 52, wherein said tethering protein further comprises an RNA-binding domain that binds to a specific sequence encoded by said nucleic acid molecule.

55. The nucleic acid molecule of claim 52, wherein said tethering protein comprises: (a) an RNA-binding domain that binds to a specific sequence encoded by said nucleic acid molecule; and (b) a transport domain that associates with a transcriptional or spliceosomal enzyme coupled to said target RNA.

56. The nucleic acid molecule of claim 55, wherein the transport domain comprises sequences isolated or derived from a gene involved in transcription, mediator complex, and/or the spliceosome.

57. The nucleic acid molecule of claim 55, wherein said RNA-binding domain that binds to said specific sequence encoded by said nucleic acid molecule comprises sequences isolated or derived from a PUF or Pumby protein.

58. The nucleic acid molecule of claim 55, wherein said RNA-binding domain that binds to said specific sequence encoded by said nucleic acid molecule comprises sequences isolated or derived from the gene SLBP.

59. The nucleic acid molecule of claim 55, wherein said tethering protein further comprises a domain that binds non-specifically to double-stranded RNA that stabilizes the RNA-RNA hybridization between said specific sequence and said target RNA.

60. The nucleic acid molecule of claim 51, wherein said tethering protein further comprises an RNA-binding domain configured to bind a specific sequence encoded by said nucleic acid molecule.

61. The nucleic acid molecule of claim 51, wherein said tethering protein is isolated or derived from human protein sequences.

62. The nucleic acid molecule of any one of claims 50-61, further comprising an engineered small nuclear RNA derived or isolated from a U1 snRNA gene that promotes trans-splicing between a target RNA and said exonic sequence.

63. The nucleic acid molecule of any one of claims 50-62, wherein said nucleic acid molecule encodes one or more binding sites for the RNA-binding domain.

64. The nucleic acid molecule of any one of claims 50-63, wherein said nucleic acid molecule further encodes a sequence that promotes accumulation of the exonic sequence molecule in the cellular nucleus.

65. The nucleic acid molecule of claim 64, wherein said sequence that promotes accumulation of the exonic sequence molecule in the cellular nucleus is derived or isolated from a long noncoding RNA.

66. The nucleic acid molecule of any one of claims 50-65, wherein said nucleic acid molecule further encodes a 3′ untranslated region that increases the stability of the trans-splicing molecule.

67. The nucleic acid molecule of any one of claims 50-66, wherein said nucleic acid molecule further encodes a 5′ untranslated region that increases the stability of the trans-splicing molecule.

68. The nucleic acid molecule of any one of claims 50-67, wherein said nucleic acid molecule further encodes a gene expression-enhancing element.

69. The nucleic acid molecule of claim 68, wherein said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element.

70. The nucleic acid molecule of any one of claims 50-69, wherein said nucleic acid molecule further encodes a heterologous promoter.

71. A vector comprising the system for trans-splicing of any one of claims 50-70.

72. The vector of claim 71, wherein said vector is selected from the group consisting of: adeno-associated virus, retrovirus, lentivirus, adenovirus, nanoparticle, micelle, liposome, lipoplex, polymersome, polyplex, and dendrimer.

73. A cell comprising the vector of claim 71.

74. A method for treating a disease comprising administering to a patient in need of a therapeutically effective amount of a treatment comprising the nucleic acid molecule according to any one of claims 50-73.

75. A method for correcting a genetic defect in a subject comprising administering to said subject the nucleic acid molecule according to any one of claims 50-74.

76. A system for trans-splicing, comprising:
a. a nucleic acid molecule encoding:
i. an exonic sequence;
ii. at least one intronic domain configured to promote insertion of said exonic sequence into a target RNA molecule; and
iii. one or more binding domains that interact with an RNA-binding protein, wherein said RNA-binding protein is encoded by one or more human-derived sequences; and
b. a tethering protein that promotes the association of said exonic sequence and said target RNA molecule, and wherein said tethering protein is configured to bind to said one or more binding domains.

77. The system for trans-splicing of claim 76, wherein said tethering protein is a fusion protein.

78. The system for trans-splicing of claim 77, wherein said tethering protein comprises: (a) an RNA-binding domain that binds to a specific sequence encoded by said nucleic acid molecule; and (b) a domain configured to associate with a transcriptional or spliceosomal protein.

79. The system for trans-splicing of claim 77, wherein said tethering protein comprises: (a) an RNA-binding domain that binds to a specific sequence encoded by said nucleic acid molecule; and (b) a non-specific double-stranded RNA binding domain that stabilizes the RNA-RNA hybridization between said specific sequence and said target RNA.

80. The system for trans-splicing of claim 79, wherein said non-specific double-stranded RNA binding domain comprises sequences isolated or derived from a gene selected from the group consisting of: DGCR8, EIF2AK2, DICER1, ILF3, ADARB1, ADAR, STA2, STAU1, PRKRA, EIF2AK2, RPS2, TRBP, CDKN2AIP, DHX9, NKRF, MRPL44, DUS2, TARBP2, DROSHA, IFIH1.

81. The system for trans-splicing of claim 79, wherein said RNA-binding domain that binds to said specific sequence encoded by said nucleic acid molecule comprises sequences isolated or derived from a PUF or Pumby protein.

82. The system for trans-splicing of claim 79, wherein said RNA-binding domain that binds to said specific sequence encoded by said nucleic acid molecule comprises sequences isolated or derived from the gene SLBP.

83. The system for trans-splicing of claim 78 or 79, wherein the tethering protein further comprises a domain that binds non-specifically to double-stranded RNA that stabilizes the RNA-RNA hybridization between said specific sequence and said target RNA.

84. The system for trans-splicing of claim 83, wherein said domain that binds non-specifically to double-stranded RNA comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1.

85. The system for trans-splicing of claim 77, wherein said tethering protein further comprises an RNA-binding domain configured to bind a specific sequence encoded by said nucleic acid molecule.

86. The system for trans-splicing of claim 77, wherein said tethering protein comprises: (a) an RNA-binding domain configured to bind a specific sequence encoded by said nucleic acid molecule; and (b) a domain configured to associate with a transcriptional or spliceosomal complex.

87. The system for trans-splicing of any one of claims 76-86, further comprising an engineered small nuclear RNA derived or isolated from a U1 snRNA gene that promotes trans-splicing between a target RNA and said exonic sequence.

88. The system for trans-splicing of any one of claims 76-86, wherein said nucleic acid molecule encodes one or more binding sites for the RNA-binding domain.

89. The system for trans-splicing of any one of claims 76-88, wherein said nucleic acid further encodes a sequence that promotes accumulation of the exonic sequence molecule in the cellular nucleus.

90. The system for trans-splicing of claim 89, wherein said sequence that promotes accumulation of the exonic sequence molecule in the cellular nucleus is derived or isolated from a long noncoding RNA.

91. The system for trans-splicing of any one of claims 76-90, wherein said nucleic acid molecule further encodes a 3′ untranslated region that increases the stability of the trans-splicing molecule.

92. The system for trans-splicing of any one of claims 76-91, wherein said nucleic acid molecule further encodes a 5′ untranslated region that increases the stability of the trans-splicing molecule.

93. The system for trans-splicing of any one of claims 76-86, wherein said nucleic acid molecule further encodes a gene expression-enhancing element.

94. The system for trans-splicing of claim 93, wherein said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element.

95. The system for trans-splicing of any one of claims 76-94, wherein said nucleic acid molecule further encodes a heterologous promoter.

96. A vector comprising the system for trans-splicing of any one of claims 76-95.

97. The vector of claim 96, wherein said vector is selected from the group consisting of: adeno-associated virus, retrovirus, lentivirus, adenovirus, nanoparticle, micelle, liposome, lipoplex, polymersome, polyplex, and dendrimer.

98. A cell comprising the vector of claim 96 or 97.

99. A method for treating a disease comprising administering to a patient in need of a therapeutically effective amount of a treatment comprising a nucleic acid molecule according to any one of claims 76-95.

100. A method for correcting a genetic defect in a subject comprising administering to said subject a nucleic acid molecule according to any one of claims 76-95.

101. A method of associating an exonic sequence with a target RNA, the method comprising:
a. providing a nucleic acid encoding:
i. an exonic sequence;
ii. at least one intronic domain configured to promote insertion of said exonic sequence into a target RNA molecule; and
iii. one or more binding domains that interact with a tethering protein; and
b. binding a tethering protein to said one or more binding domains and to said target RNA molecule to associate said exonic sequence with, said target RNA molecule.

102. The method of claim 101, wherein said method is performed in the absence of a CRISPR-associated enzyme.

103. The method of claim 101, wherein said tethering protein is a fusion protein.

104. The method of claim 102, wherein said tethering protein comprises: (a) an RNA-binding domain that binds to a specific sequence encoded by said nucleic acid molecule; and (b) a domain configured to associate with a transcriptional or spliceosomal protein.

105. The method of claim 102, wherein said tethering protein comprises: (a) an RNA-binding domain that binds to a specific sequence encoded by said nucleic acid molecule; and (b) a non-specific double-stranded RNA binding domain that stabilizes the RNA-RNA hybridization between said specific sequence and said target RNA.

106. The method of claim 105, wherein said non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1.

107. The method of claim 105, wherein said RNA-binding domain that binds to said specific sequence encoded by said nucleic acid molecule comprises sequences isolated or derived from a PUF or Pumby protein.

108. The method of claim 105, wherein said RNA-binding domain that binds to said specific sequence encoded by said nucleic acid molecule comprises sequences isolated or derived from the gene SLBP.

109. The method of claim 102, further comprising providing a enzyme configured to insert said exonic sequence into said target RNA molecule.

110. The method of claim 102, wherein said tethering protein further comprises a doming configured to associate with said enzyme configured to insert said exonic sequence into said target RNA molecule.

111. The method of claim 102, wherein said tethering protein further comprises an RNA-binding domain configured to bind a specific sequence encoded by said nucleic acid molecule.

112. The method of claim 102, wherein said tethering protein comprises: (a) an RNA-binding domain configured to bind a specific sequence encoded by said nucleic acid molecule; and (b) a domain configured to associate with a transcriptional or spliceosomal complex.

113. The method of claim 102, wherein said tethering protein is isolated or derived from human protein sequences.

114. The method of any one of claims 101-113, further comprising providing an engineered small nuclear RNA derived or isolated from a U1 snRNA gene that promotes trans-splicing between a target RNA and said exonic sequence.

115. The method of any one of claims 101-114, wherein said nucleic acid molecule further encodes a sequence that promotes accumulation of the exonic sequence molecule in the cellular nucleus.

116. The method of claim 115, wherein said sequence that promotes accumulation of the exonic sequence molecule in the cellular nucleus is derived or isolated from a long noncoding RNA.

117. The method of any one of claims 101-116, wherein said nucleic acid molecule further encodes a 3′ untranslated region that increases the stability of the trans-splicing molecule.

118. The method of any one of claims 101-117, wherein said nucleic acid molecule further encodes a 5′ untranslated region that increases the stability of the trans-splicing molecule.

119. The method of any one of claims 101-118, wherein said nucleic acid molecule further encodes a gene expression-enhancing element.

120. The method of claim 119, wherein said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element.

121. The method of any one of claims 101-120, wherein said nucleic acid molecule further encodes a heterologous promoter.

122. A method of associating an exonic sequence with a target RNA, the method comprising:
a. providing a nucleic acid molecule encoding:
i. a exonic sequence; and
ii. at least one intronic domain configured to promote insertion of said exonic sequence into a target RNA molecule; and
b. binding a tethering protein to said target RNA molecule and said exonic sequence to associate said exonic sequence with said target RNA molecule, wherein said trans-splicing molecule does not associate with a CRISPR enzyme.

123. The method of claim 122, wherein said tethering protein is a fusion tethering protein.

124. The method of claim 122 or 123, wherein said tethering protein comprises: (a) an RNA-binding domain that binds to a specific sequence encoded by said nucleic acid molecule; and (b) a domain configured to associate with a transcriptional or spliceosomal protein.

125. The method of any one of claims 122-124, wherein said tethering protein comprises: (a) an RNA-binding domain, that binds to a specific sequence encoded by said nucleic acid molecule; and (b) a non-specific double-stranded RNA binding domain that stabilizes the RNA-RNA hybridization between said specific sequence and said target RNA.

126. The method of claim 125, wherein said non-specific double-stranded RNA binding domain comprises sequences isolated or derived from a gene selected from the group consisting of DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1.

127. The method of claim 125, wherein said RNA-binding domain that binds to said specific sequence encoded by said nucleic acid molecule comprises sequences isolated or derived from a PUF or Pumby protein.

128. The method of claim 125, wherein said RNA-binding domain that binds to said specific sequence encoded by said nucleic acid molecule comprises sequences isolated or derived from the gene SLBP.

129. The method of any one of claims 122-128, further comprising providing a enzyme configured to insert said exonic sequence into said target RNA molecule.

130. The method of any one of claims 122-129, wherein said tethering protein further comprises a domain configured to associate with said enzyme configured to insert said exonic sequence into said target RNA molecule.

131. The method of any one of claims 122-130, wherein said tethering protein comprises: (a) an RNA-binding domain configured to bind a specific sequence encoded by said nucleic acid molecule; and (b) a domain configured to associate with a transcriptional or spliceosomal complex.

132. The method of any one of claims 122-131, wherein said tethering protein is isolated or derived from human protein sequences.

133. The method of any one of claims 122-132, further comprising providing an engineered small nuclear RNA derived or isolated from a U1 snRNA gene that promotes trans-splicing between a target RNA and said exonic sequence.

134. The method of any one of claims 122-133, wherein said nucleic acid molecule encodes one or more binding sites for the RNA-binding domain.

135. The method of any one of claims 122-134, wherein said nucleic acid molecule further encodes a sequence that promotes accumulation of the exonic sequence molecule in the cellular nucleus.

136. The method of claim 135, wherein said sequence that promotes accumulation of the exonic sequence molecule in the cellular nucleus is derived or isolated from a long noncoding RNA.

137. The method of any one of claims 122-136, wherein said nucleic acid molecule further encodes a 3′ untranslated region that increases the stability of the trans-splicing molecule.

138. The method of any one of claims 122-137, wherein said nucleic acid molecule further encodes a 5′ untranslated region that increases the stability of the trans-splicing molecule.

139. The method of any one of claims 122-138, wherein said nucleic acid molecule further encodes a gene expression-enhancing element.

140. The method of claim 139, wherein said gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element.

141. The method of any one of claims 122-140, wherein said nucleic acid molecule further encodes a heterologous promoter.

142. A system for trans-splicing comprising a nucleic acid encoding an exonic sequence and a tethering fusion protein, wherein the tethering fusion protein promotes the association of the exonic sequence and a target RNA.

143. The system for trans-splicing of claim 142, wherein the tethering fusion protein comprises: (a) an RNA-binding domain that binds to a specific sequence encoded by the nucleic acid molecule; and (b) a non-specific double-stranded RNA binding domain that stabilizes the RNA-RNA hybridization among the specific sequence encoded by the nucleic acid molecule and the target RNA.

144. The system for trans-splicing of claim 143, wherein the non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH1.

145. The system for trans-splicing of claim 143, wherein the RNA-binding domain that binds to a specific sequence encoded by the nucleic acid molecule comprises sequences isolated or derived from a PUF or Pumby protein.

146. The system for trans-splicing of claim 143, wherein the RNA-binding domain that binds to a specific sequence encoded by the nucleic acid molecule comprises sequences isolated or derived from the gene SLBP.

147. The system for trans-splicing of claim 142, wherein the tethering fusion protein comprises: (a) an RNA-binding domain that binds a specific sequence encoded by said nucleic acid molecule; and (b) a domain that associates with the spliceosome.

148. The system for trans-splicing of claim 142, wherein the tethering fusion protein comprises: (a) an RNA-binding domain that binds a specific sequence encoded by said nucleic acid molecule; and (b) a domain that associates with a transcriptional or spliceosomal complex.

149. The system for trans-splicing of any one of claims 142-148, wherein the tethering fusion protein is isolated or derived from human protein sequences.

150. The system for trans-splicing of any one of claims 142-149, further comprising an engineered small nuclear RNA derived or isolated from the U1 snRNA gene that promotes trans-splicing among the target RNA and exonic sequence.

151. The system for trans-splicing of any one of claims 142-150, wherein the nucleic acid molecule encodes one or more binding sites for the RNA-binding domain.

152. The system for trans-splicing of any one of claims 142-151, wherein the exonic sequence further comprises a sequence promotes accumulation of the exonic sequence in the cellular nucleus.

153. The system for trans-splicing of claim 152, wherein the sequence that promotes accumulation of the exonic sequence in the cellular nucleus is derived or isolated from a long noncoding RNA.

154. The system for trans-splicing of any one of claims 142-153, wherein the nucleic acid molecule further encodes a 3′ untranslated region that increases the stability of the trans-splicing molecule.

155. The system for trans-splicing of any one of claims 142-154, wherein the nucleic acid molecule further encodes a 5′ untranslated region that increases the stability of the trans-splicing molecule.

156. The system for trans-splicing of any one of claims 142-155, wherein the nucleic acid molecule further encodes a gene expression-enhancing element.

157. The system for trans-splicing of claim 156, wherein the gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element.

158. The system for trans-splicing of any one of claims 142-157, wherein the nucleic acid molecule comprises RNA, DNA, a DNA RNA hybrid, a nucleic acid analog, a chemically-modified nucleic acid, or a chimera composed of two or more nucleic acids or nucleic acid analogs.

159. The system for trans-splicing of any one of claims 142-158, wherein the nucleic acid molecule further comprises a heterologous promoter.

160. A vector comprising the system for trans-splicing of any one of claims 142-160.

161. The vector of claim 160, wherein the vector is selected from the group consisting of: adeno-associated virus, retrovirus, lentivirus, adenovirus, nanoparticle, micelle, liposome, lipoplex, polymersome, polyplex, and dendrimer.

162. A cell comprising the vector of claim 160 or 161.

163. A method for treating a disease comprising administering to a patient in need of a therapeutically effective amount of a treatment comprising a nucleic acid molecule according to any one of claims 142-160.

164. A method for correcting a genetic defect in a subject comprising administering to said subject a nucleic acid molecule according to any one of claims 142-160.

165. A method of targeting an exonic sequence to a target RNA, the method comprising:
a. providing a nucleic acid encoding said exonic sequence;
b. providing said target RNA; and
c. using a tethering fusion protein to associate said exonic sequence and said target RNA.

166. The method of claim 165, wherein the tethering fusion protein comprises: (a) an RNA-binding domain that binds to a specific sequence encoded by the nucleic acid molecule; and (b) a non-specific double-stranded RNA binding domain that stabilizes the RNA-RNA hybridization among the specific sequence and the target RNA.

167. The method of claim 166, wherein the non-specific double-stranded RNA binding domain comprises sequences isolated or derived from a gene selected from the group consisting of: DGCR8, EIF2AK2, DICER1, ILF3, ADARB1, ADAR, STAU2, STAU1, PRKRA, EIF2AK2, RPS2, TRBP, CDKN2AIP, DHX9, NKRF, MRPL44, DUS2, TARBP2, DROSHA, IFIH14.

168. The method of claim 166, wherein the RNA-binding domain that binds to a specific sequence encoded by the nucleic acid molecule comprises sequences isolated or derived from a PUF or Pumby protein.

169. The method of claim 166, wherein the RNA-binding domain that binds to a specific sequence encoded by the nucleic acid molecule comprises sequences isolated or derived from the gene SLBP.

170. The method of any one of claims 165-169, wherein the tethering fusion protein comprises: (a) an RNA-binding domain that binds a specific sequence encoded by said nucleic acid molecule; and (b) a domain that associates with the spliceosome.

171. The method of any one of claims 165-169, wherein the tethering fusion protein comprises: (a) an RNA-binding domain that binds a specific sequence encoded by said nucleic acid molecule; and (b) a domain that associates with a transcriptional or spliceosomal complex.

172. The method of any one of claims 165-171, wherein the tethering fusion protein is isolated or derived from human protein sequences.

173. The method of any one of claims 165-172, further comprising an engineered small nuclear RNA derived or isolated from the U1 snRNA gene that promotes trans-splicing among the target RNA and exonic sequence.

174. The method of any one of claims 165-173, wherein the nucleic acid molecule encodes one or more binding sites for the RNA-binding domain.

175. The method of any one of claims 165-174, wherein the exonic sequence further comprises a sequence promotes accumulation of the exonic sequence in the cellular nucleus.

176. The method of claim 175, wherein the sequence that promotes accumulation of the exonic sequence in the cellular nucleus is derived or isolated from a long noncoding RNA.

177. The method of any one of claims 165-176, wherein the nucleic acid molecule further encodes a 3′ untranslated region that increases the stability of the trans-splicing molecule.

178. The method of any one of claims 165-177, wherein the nucleic acid molecule further encodes a 5′ untranslated region that increases the stability of the trans-splicing molecule.

179. The method of any one of claims 165-178, wherein the nucleic acid molecule further encodes a gene expression-enhancing element.

180. The method of claim 179, wherein the gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element.

181. The method of any one of claims 165-180, wherein the nucleic acid molecule comprises RNA, DNA, a DNA/RNA hybrid, a nucleic acid analog, a chemically-modified nucleic acid, or a chimera composed of two or more nucleic acids or nucleic acid analogs.

182. The method of any one of claims 165-181, wherein the nucleic acid molecule further comprises a heterologous promoter.
